(19) United States
(12) Patent Application Publication
(10) Pub. No.: US 2008/0280859 A1
(43) Pub. Date: Nov. 13, 2008

(54) GEMINI VITAMIN D3 COMPOUNDS AND METHODS OF USE THEREOF

(75) Inventors: Luciano Adorini, Milan (IT); Giuseppe Penna, Cusano Milanino (IT); Milan R. Uskokovic, Upper Montclair, NJ (US); Hubert Maehr, Wayne, NJ (US)

Correspondence Address: EDWARDS ANGELL PALMER & DODGE LLP, P.O. BOX 55874, BOSTON, MA, 02205, US

(73) Assignee: BioXell S.p.A., Milan (IT)

(21) Appl. No.: 10/554,038

(22) Filed: Oct. 19, 2005

(51) Int. Cl. A61K 31/593; C07C 401/00; A61P 35/00; A61P 13/00; A61P 37/06

(52) U.S. Cl. 514/167; 552/653

(57) ABSTRACT

The invention provides Gemini vitamin D₃ compounds, methods for using the compounds to treat vitamin D3 associated states and pharmaceutical compositions containing the compounds.

### RELATED APPLICATION

This application claims the benefit of U.S. provisional patent application Ser. No. 60/466,638 filed Apr. 30, 2003, the disclosure of which application is incorporated herein in its entirety by this reference.

### BACKGROUND OF THE INVENTION

The importance of vitamin D (cholecalciferol) in the biological systems of higher animals has been recognized since its discovery by Mellanby in 1920 (Mellanby, E. (1921) Spec. Rep. Ser. Med. Res. Council (GB) SRS 61:4). It was in the interval of 1920-1930 that vitamin D officially became classified as a “vitamin” that was essential for the normal development of the skeleton and maintenance of calcium and phosphorous homeostasis.

Studies involving the metabolism of vitamin D₃ were initiated with the discovery and chemical characterization of the plasma metabolite, 25-hydroxyvitamin D₃ [25(OH)D₃] (Blunt, J. W. et al. (1968) Biochemistry 6:3317-3322) and the hormonally active form, 1α,25(OH)₂D₃ (Myrtle, J. F. et al. (1970) J. Biol. Chem. 245:1190-1196; Norman, A. W. et al. (1971) Science 173:51-54; Lawson, D. E. M. et al. (1971) Nature 230:228-230; Holick, M. F. (1971) Proc. Natl. Acad. Sci. USA 68:803-804). The formulation of the concept of a vitamin D endocrine system was dependent both upon appreciation of the key role of the kidney in producing 1α,25(OH)₂D₃ in a carefully regulated fashion (Fraser, D. R. and Kodicek, E (1970) Nature 288:764-766; Wong, R. G. et al. (1972) J. Clin. Invest. 51:1287-1291), and the discovery of a nuclear receptor for 1α,25(OH)₂D₃ (VD₃R) in the intestine (Haussler, M. R. et al. (1969) Exp. Cell Res. 58:234-242; Tsai, H. C. and Norman, A. W. (1972) J. Biol. Chem. 248:5967-5975).

The operation of the vitamin D endocrine system depends on the following: first, on the presence of cytochrome P450 enzymes in the liver (Bergman, T. and Postlind, H. (1991) Biochem. J. 276:427-432; Ohyama, Y. and Okuda, K. (1991) J. Biol. Chem. 266:8690-8695) and kidney (Henry, H. L. and Norman, A. W. (1974) J. Biol. Chem. 249:7529-7535; Gray, R. W. and Ghazarian, J. G. (1989) Biochem. J. 259:561-568), and in a variety of other tissues to effect the conversion of vitamin D₃ into biologically active metabolites such as 1α, 25(OH)₂D₃ and 24R,25(OH)₂D₃; second, on the existence of the plasma vitamin D binding protein (DBP) to effect the selective transport and delivery of these hydrophobic molecules to the various tissue components of the vitamin D endocrine system (Van Baelen, H. et al. (1988) Ann NY Acad. Sci. 538:60-68; Cooke, N. E. and Haddad, J. G. (1989) Enidocr. Rev. 10:294-307; Bikle, D. D. et al. (1986) J. Clin. Endoctinol. Metab. 63:954-959); and third, upon the existence of stereoselective receptors in a wide variety of target tissues that interact with the agonist 1α,25(OH)₂D₃ to generate the requisite specific biological responses for this secosteroid hormone (Pike, J. W. (1991) Annu. Rev. Nutr. 11:189-216). To date, there is evidence that nuclear receptors for 1α,25(OH)₂D₃ (VD₃R) exist in more than 30 tissues and cancer cell lines (Reichel, H. and Norman, A. W. (1989) Anu. Rev. Med. 40:71-78).

Vitamin D₃ and its hormonally active forms are well-known regulators of calcium and phosphorous homeostasis. These compounds are known to stimulate, at least one of, intestinal absorption of calcium and phosphate, mobilization of bone mineral, and retention of calcium in the kidneys. Furthermore, the discovery of the presence of specific vitamin D receptors in more than 30 tissues has led to the identification of vitamin D₃ as a pluripotent regulator outside its classical role in calcium/bone homeostasis. A paracrine role for 1α,25(OH)₂ D₃ has been suggested by the combined presence of enzymes capable of-oxidizing vitamin D₃ into its active forms, e.g., 25-OHD-1α-hydroxylase, and specific receptors in several tissues such as bone, keratinocytes, placenta, and immune cells. Moreover, vitamin D₃ hormone and active metabolites have been found to be capable of regulating cell proliferation and differentiation of both normal and malignant cells (Reichel, H. et al. (1989) Ann. Rev. Med. 40:71-78).

Given the activities of vitamin D₃ and its metabolites, much attention has focused on the development of synthetic analogs of these compounds. A large number of these analogs involve structural modifications in the A ring, B ring, C/D rings, and, primarily, the side chain (Bouillon, R. et al., Endocrine Reviews 16(2):201-204). Although a vast majority of the vitamin D₃ analogs developed to date involve structural modifications in the side chain, a few studies have reported the biological profile of A-ring diastereomers (Norman, A. W. et al. J. Biol. Chem. 268 (27): 20022-20030). Furthermore, biological esterification of steroids has been studied (Hochberg, R. B., (1998) Endocr Rev. 19(3): 331-348), and esters of vitamin D₃ are known (WO 97/11053).

Moreover, despite much effort in developing synthetic analogs, clinical applications of vitamin D and its structural analogs have been limited by the undesired side effects elicited by these compounds after administration to a subject for known indications/applications of vitamin D compounds.

### SUMMARY OF THE INVENTION

The invention provides vitamin D₃ compounds having formula I:

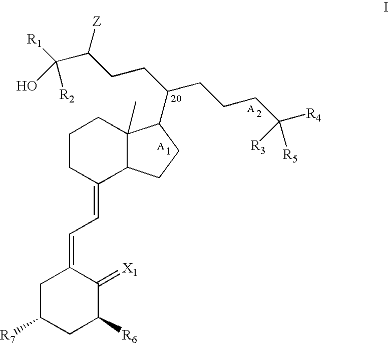

wherein:

A₁ is a single or double bond;

A₂ is a single, a double or a triple bond;

R₁, R₂, R₃ and R₄ are each independently C₁-C₄ alkyl, C₁-C₄ deuteroalkyl, hydroxyalkyl, or haloalkyl;

R₅, R₆ and R₇ are each independently hydroxyl, OC(O)C₁-C₄ alkyl, OC(O)hydroxyalkyl, or OC(O)haloalkyl;

the configuration at C₂₀ is R or S;

X₁ is H₂ or CH₂;

Z is hydrogen when at least one of R₁ and R₂ is C₁-C₄ deuteroalkyl and at least one of R₃ and R₄ is haloalkyl or when at least one of R₁ and R₂ is haloalkyl and at least one of R₃ and R₄ is C₁-C₄ deuteroalkyl; or Z is —OH, ═O, —SH, or —NH₂;

and pharmaceutically acceptable esters, salts, and prodrugs thereof.

The invention also provides methods for treating a subject for a vitamin D₃ associated state, by administering to the subject an effective amount of a vitamin D₃ compound of formula I above or otherwise described herein.

Another embodiment of the invention provides a method for treating a subject for a urogenital disorder, comprising administering to the subject an effective amount of a vitamin D₃ compound of formula I above or otherwise described herein, such that said subject is treated for the urogential disorder.

In another embodiment, the invention provides a method of treating an ILT3-associated disorder in a subject. The method includes administering to the subject a vitamin D₃ compound of formula I above or otherwise described herein, in an amount effective to modulate the expression of an ILT3 surface molecule.

In yet another embodiment, the invention provides a method of inhibiting transplant rejection in a subject. The method includes administering to the subject a vitamin D₃ compound of formula I above or otherwise described herein in an amount effective to modulate the expression of an ILT3 surface molecule.

The invention also provides a method for treating a subject for hypertension, comprising administering to the subject an effective amount of a Gemini vitamin D₃ compound of the invention, such that the subject is treated for hypertension. In a related embodiment, the invention provides a method of suppressing renin expression in a subject comprising administering a to a subject an effective amount of a Gemini vitamin D₃ compound such that renin expression in said subject is suppressed.

In another embodiment, the invention also provides a method of ameliorating a deregulation of calcium and phosphate metabolism. The method includes administering to a subject a therapeutically effective amount of a vitamin D₃ compound of formula I or otherwise described herein, so as to ameliorate the deregulation of the calcium and phosphate metabolism.

In a further embodiment, the invention provides a method of modulating the expression of an immunoglobulin-like transcript 3 (ILT3) surface molecule in a cell. The method includes contacting the cell with a vitamin D₃ compound of formula I or otherwise described herein, in an amount effective to modulate the expression of an immunoglobulin-like transcript 3 (ILT3) surface molecule in the cell.

In another embodiment, the invention provides a method of inducing immunological tolerance in a subject, by administering to the subject a vitamin D₃ compound of formula I or otherwise described herein, in an amount effective to modulate the expression of an ILT3 surface molecule, to thereby induce immunological tolerance in the subject.

In yet another embodiment, the invention provides a method for modulating immunosuppressive activity by an antigen-presenting cell, by contacting an antigen-presenting cell with a vitamin D₃ compound of formula I or otherwise described herein, in an amount effective to modulate ILT3 surface molecule expression, to thereby modulating immunosuppressive activity by an antigen-presenting cell.

The invention also provides a pharmaceutical composition, comprising an effective amount a vitamin D₃ compound of formula I or otherwise described herein and a pharmaceutically acceptable carrier.

In another embodiment, the invention provides a packaged formulation which includes a pharmaceutical composition comprising a vitamin D₃ compound of formula I or otherwise described herein, and a pharmaceutically-acceptable carrier packaged with instructions for use in the treatment of a for use in the treatment of a vitamin D₃ associated state.

### BRIEF DESCRIPTION OF THE DRAWINGS

FIG. 1 is a graph depicting the modulation (upregulation) of expression of ILT3 on the cell surface of monocyte-derived immature dendritic cells with various compounds.

### DETAILED DESCRIPTION OF THE INVENTION

### 1. Definitions

Before further description of the present invention, and in order that the invention may be more readily understood, certain terms are first defined and collected here for convenience.

The term “administration” or “administering” includes routes of introducing the vitamin D₃ compound(s) to a subject to perform their intended function. Examples of routes of administration which can be used include injection (subcutaneous, intravenous, parenterally, intraperitoneally, intrathecal), oral, inhalation, rectal and transdermal. The pharmaceutical preparations are, of course, given by forms suitable for each administration route. For example, these preparations are administered in tablets or capsule form, by injection, inhalation, eye lotion, ointment, suppository, etc. administration by injection, infusion or inhalation; topical by lotion or ointment; and rectal by suppositories. Oral administration is preferred. The injection can be bolus or can be continuous infusion. Depending on the route of administration, the vitamin D₃ compound can be coated with or disposed in a selected material to protect it from natural conditions which may detrimentally effect its ability to perform its intended function. The vitamin D₃ compound can be administered alone, or in conjunction with either another agent as described above or with a pharmaceutically-acceptable carrier, or both. The vitamin D₃ compound can be administered prior to the administration of the other agent, simultaneously with the agent, or after the administration of the agent. Furthermore, the vitamin D₃ compound can also be administered in a proform which is converted into its active metabolite, or more active metabolite in vivo.

The term “alkyl” refers to the radical of saturated aliphatic groups, including straight-chain alkyl groups, branched-chain alkyl groups, cycloalkyl (alicyclic) groups, alkyl substituted cycloalkyl groups, and cycloalkyl substituted alkyl groups. The term alkyl further includes alkyl groups, which can further include oxygen, nitrogen, sulfur or phosphorous atoms replacing one or more carbons of the hydrocarbon backbone, e.g., oxygen, nitrogen, sulfur or phosphorous atoms. In preferred embodiments, a straight chain or branched chain alkyl has 30 or fewer carbon atoms in its backbone (e.g., C₁-C₃₀ for straight chain, C₃-C₃₀ for branched chain), preferably 26 or fewer, and more preferably 20 or fewer. Likewise, preferred cycloalkyls have from 3-10 carbon atoms in their ring structure, and more preferably have 3, 4, 5, 6 or 7 carbons in the ring structure.

Moreover, the term alkyl as used throughout the specification and claims is intended to include both “unsubstituted alkyls” and “substituted alkyls,” the latter of which refers to alkyl moieties having substituents replacing a hydrogen on one or more carbons of the hydrocarbon backbone. Such substituents can include, for example, halogen, hydroxyl, alkylcarbonyloxy, arylcarbonyloxy, alkoxycarbonyloxy, aryloxycarbonyloxy, carboxylate, alkylcarbonyl, alkoxycarbonyl, aminocarbonyl, alkylthiocarbonyl, alkoxyl, phosphate, phosphonato, phosphinato, cyano, amino (including alkyl amino, dialkylamino, arylamino, diarylamino, and alkylarylamino), acylamino (including alkylcarbonylamino, arylcarbonylamino, carbamoyl and ureido), amidino, imino, sulfhydryl, alkylthio, arylthio, thiocarboxylate, sulfates, sulfonato, sulfamoyl, sulfonamido, nitro, trifluoromethyl, cyano, azido, heterocyclyl, alkylaryl, or an aromatic or heteroaromatic moiety. It will be understood by those skilled in the art that the moieties substituted on the hydrocarbon chain can themselves be substituted, if appropriate. Cycloalkyls can be further substituted, e.g., with the substituents described above. An “alkylaryl” moiety is an alkyl substituted with an aryl (e.g., phenylmethyl (benzyl)). The term “alkyl” also includes unsaturated aliphatic groups analogous in length and possible substitution to the alkyls described above, but that contain at least one double or triple bond respectively.

Unless the number of carbons is otherwise specified, “lower alkyl” as used herein means an alkyl group, as defined above, but having from one to ten carbons, more preferably from one to six, and most preferably from one to four carbon atoms in its backbone structure, which may be straight or branched-chain. Examples of lower alkyl groups include methyl, ethyl, n-propyl, i-propyl, tert-butyl, hexyl, heptyl, octyl and so forth. In preferred embodiment, the term “lower alkyl” includes a straight chain alkyl having 4 or fewer carbon atoms in its backbone, e.g., C₁-C₄ alkyl.

The terms “alkoxyalkyl,” “polyaminoalkyl” and “thioalkoxyalkyl” refer to alkyl groups, as described above, which further include oxygen, nitrogen or sulfur atoms replacing one or more carbons of the hydrocarbon backbone, e.g., oxygen, nitrogen or sulfur atoms.

The terms “alkenyl” and “alkynyl” refer to unsaturated aliphatic groups analogous in length and possible substitution to the alkyls described above, but that contain at least one double or triple bond, respectively. For example, the invention contemplates cyano and propargyl groups.

The term “antigen” includes a substance which elicits an immune response. The antigens of the invention to which tolerance is induced may or may not be exogenously derived relative to the host. For example, the method of the invention may be used to induce tolerance to an “autoantigen.” An autoantigen is a normal constituent of the body that reacts with an autoantibody. The invention also includes inducing tolerance to an “alloantigen.” Alloantigen refers to an antigen found only in some members of a species, for example the blood group substances. An allograft is a graft to a genetically different member of the same species. Allografts are rejected by virtue of the immunological response of T lymphocytes to histocompatibility antigens. The method of the invention also provides for inducing tolerance to a “xenoantigen.” Xenoantigens are substances that cause an immune reaction due to differences between different species. Thus, a xenograft is a graft from a member of one species to a member of a different species. Xenografts are usually rejected within a few days by antibodies and cytotoxic T lymphocytes to histocompatibility antigens.

The language “antigen-presenting cell” or “APC” includes a cell that is able to present an antigen to, for example, a T helper cell. Antigen-presenting cells include B lymphocytes, accessory cells or non-lymphocytic cells, such as dendritic cells, Langerhans cells, and mononuclear phagocytes that help in the induction of an immune response by presenting antigen to helper T lymphocytes. The antigen-presenting cell of the present invention is preferably of myeloid origin, and includes, but is not limited to, dendritic cells, macrophages, monocytes. APCs of the present invention may be isolated from the bone marrow, blood, thymus, epidermis, liver, fetal liver, or the spleen.

The terms “antineoplastic agent” and “antiproliferative agent” are used interchangeably herein and includes agents that have the functional property of inhibiting the proliferation of a vitamin D₃-responsive cells, e.g., inhibit the development or progression of a neoplasm having such a characteristic, particularly a hematopoietic neoplasm.

The term “aryl” as used herein, refers to the radical of aryl groups, including 5- and 6-membered single-ring aromatic groups that may include from zero to four heteroatoms, for example, benzene, pyrrole, furan, thiophene, imidazole, benzoxazole, benzothiazole, triazole, tetrazole, pyrazole, pyridine, pyrazine, pyridazine and pyrimidine, and the like. Aryl groups also include polycyclic fused aromatic groups such as naphthyl, quinolyl, indolyl, and the like. Those aryl groups having heteroatoms in the ring structure may also be referred to as “aryl heterocycles,” “heteroaryls” or “heteroaromatics.” The aromatic ring can be substituted at one or more ring positions with such substituents as described above, as for example, halogen, hydroxyl, alkoxy, alkylcarbonyloxy, arylcarbonyloxy, alkoxycarbonyloxy, aryloxycarbonyloxy, carboxylate, alkylcarbonyl, alkoxycarbonyl, aminocarbonyl, alkylthiocarbonyl, phosphate, phosphonato, phosphinato, cyano, amino (including alkyl amino, dialkylamino, arylamino, diarylamino, and alkylarylamino), acylamino (including alkylcarbonylamino, arylcarbonylamino, carbamoyl and ureido), amidino, imino, sulfhydryl, alkylthio, arylthio, thiocarboxylate, sulfates, sulfonato, sulfamoyl, sulfonamido, nitro, trifluoromethyl, cyano, azido, heterocyclyl, alkaryl, or an aromatic or heteroaromatic moiety. Aryl groups can also be fused or bridged with alicyclic or heterocyclic rings which are not aromatic so as to form a polycycle (e.g., tetralin).

The language “autoimmune disease” or “autoimmune disorder” refers to the condition where the immune system attacks the host's own tissue(s). In an autoimmune disease, the immune tolerance system of the patient fails to recognize self antigens and, as a consequence of this loss of tolerance, brings the force of the immune system to bear on tissues which express the antigen. Autoimmune disorders include, but are not limited to, type 1 insulin-dependent diabetes mellitus, adult respiratory distress syndrome, inflammatory bowel disease, dermatitis, meningitis, thrombotic thrombocytopenic purpura, Sjogren's syndrome, encephalitis, uveitic, leukocyte adhesion deficiency, rheumatoid arthritis, rheumatic fever, Reiter's syndrome, psoriatic arthritis, progressive systemic sclerosis, primary biliary cirrhosis, pemphigus, pemphigoid, necrotizing vasculitis, myasthenia gravis, multiple sclerosis, lupus erythematosus, polymyositis, sarcoidosis, granulomatosis, vasculitis, pernicious anemia, CNS inflammatory disorder, antigen-antibody complex mediated diseases, autoimmune haemolytic anemia, Hashimoto's thyroiditis, Graves disease, habitual spontaneous abortions, Reynard's syndrome, glomerulonephritis, dermatomyositis, chronic active hepatitis, celiac disease, autoimmune complications of AIDS, atrophic gastritis, ankylosing spondylitis and Addison's disease.

The language “biological activities” of vitamin D₃ includes all activities elicited by vitamin D₃ compounds in a responsive cell. It includes genomic and non-genomic activities elicited by these compounds (Gniadecki W and Calverley M. J. (1998) Pharmacology & Toxicology 82:173-176; Bouillon, R. et al. (1995) Endocrinology Reviews 16(2):206-207; Norman A. W. et al. (1992) J. Steroid Biochem Mol. Biol. 41:231-240; Baran D. T. et al. (1991) J. Bone Miner Res. 6:1269-1275; Caffrey J. M. and Farach-Carson M. C. (1989) J. Biol. Chem. 264:20265-20274; Nemere I. et al. (1984) Endocrinology 115:1476-1483).

The language “bone metabolism” includes direct or indirect effects in the formation or degeneration of bone structures, e.g., bone formation, bone resorption, etc., which may ultimately affect the concentrations in serum of calcium and phosphate. This term is also intended to include effects of compounds of the invention in bone cells, e.g., osteoclasts and osteoblasts, that may in turn result in bone formation and degeneration.

The language “calcium and phosphate homeostasis” refers to the careful balance of calcium and phosphate concentrations, intracellularly and extracellularly, triggered by fluctuations in the calcium and phosphate concentration in a cell, a tissue, an organ or a system. Fluctuations in calcium levels that result from direct or indirect responses to compounds of the invention are intended to be included by these terms.

The term “carcinoma” is art recognized and refers to malignancies of epithelial or endocrine tissues including respiratory system carcinomas, gastrointestinal system carcinomas, genitourinary system carcinomas, testicular carcinomas, breast carcinomas, prostatic carcinomas, endocrine system carcinomas, and melanomas. Exemplary carcinomas include those forming from tissue of the cervix, lung, prostate, breast, head and neck, colon and ovary. The term also includes carcinosarcomas, e.g., which include malignant tumors composed of carcinomatous and sarcomatous tissues. An “adenocarcinoma” refers to a carcinoma derived from glandular tissue or in which the tumor cells form recognizable glandular structures.

The term “chiral” refers to molecules which have the property of non-superimposability of the mirror image partner, while the term “achiral” refers to molecules which are superimposable on their mirror image partner.

The term “diastereomers” refers to stereoisomers with two or more centers of dissymmetry and whose molecules are not mirror images of one another.

The term “deuteroalkyl” refers to alkyl groups in which one or more of the of the hydrogens has been replaced with deuterium.

The term “effective amount” includes an amount effective, at dosages and for periods of time necessary, to achieve the desired result, e.g., sufficient treat a vitamin D₃ associated state or to modulate ILT3 expression in a cell. An effective amount of vitamin D₃ compound may vary according to factors such as the disease state, age, and weight of the subject, and the ability of the vitamin D₃ compound to elicit a desired response in the subject. Dosage regimens may be adjusted to provide the optimum therapeutic response. An effective amount is also one in which any toxic or detrimental effects (e.g., side effects) of the angiogenesis inhibitor compound are outweighed by the therapeutically beneficial effects.

A therapeutically effective amount of vitamin D₃ compound (i.e., an effective dosage) may range from about 0.001 to 30 μg/kg body weight, preferably about 0.01 to 25 μg/kg body weight, more preferably about 0.1 to 20 μg/kg body weight, and even more preferably about 1 to 10 μg/kg, 2 to 9 μg/kg, 3 to 8 μg/kg, 4 to 7 μg/kg, or 5 to 6 μg/kg body weight. The skilled artisan will appreciate that certain factors may influence the dosage required to effectively treat a subject, including but not limited to the severity of the disease or disorder, previous treatments, the general health and/or age of the subject, and other diseases present. Moreover, treatment of a subject with a therapeutically effective amount of a vitamin D₃ compound can include a single treatment or, preferably, can include a series of treatments. In one example, a subject is treated with a vitamin D₃ compound in the range of between about 0.1 to 20 μg/kg body weight, one time per week for between about 1 to 10 weeks, preferably between 2 to 8 weeks, more preferably between about 3 to 7 weeks, and even more preferably for about 4, 5, or 6 weeks. It will also be appreciated that the effective dosage of a vitamin D₃ compound used for treatment may increase or decrease over the course of a particular treatment.

The term “enantiomers” refers to two stereoisomers of a compound which are non-superimposable mirror images of one another. An equimolar mixture of two enantiomers is called a “racemic mixture” or a “racemate.”

The language “Gemin vitamin D₃ compounds” is intended to include vitamin D₃ compounds and analogs thereof having bis C20 side chains. Vitamin D₃ compounds are characterized by an “A” ring (monocycle) which is connected to a “B” ring (bicycle) which is connected to a side chain at carbon C20 of the side chain. The Gemini compounds of the invention have two side chains and are, therefore, conspicuously distinguishable from vitamin D₃ compounds having a single side chain. Candidate A and B rings for the Gemini compounds of the invention are disclosed in U.S. Pat. Nos. 6,559,138, 6,329,538, 6,331,642, 6,452,028, 6,492,353, 6,040,461, 6,030,963, 5,939,408, 5,872,113, 5,840,718, 5,612,328, 5,512,554, 5,451,574, 5,428,029, 5,145,846, and 4,225,525. Examples of Gemini compounds in accordance with the invention are disclosed in U.S. Pat. No. 6,030,962.

The language “genomic” activities or effects of vitamin D₃ is intended to include those activities mediated by the nuclear receptor for 1α, 25(OH)₂D₃ (VD₃R), e.g., transcriptional activation of target genes.

The term “halogen” designates —F, —Cl, —Br or —I.

The term “haloalkyl” is intended to include alkyl groups as defined above that are mono-, di- or polysubstituted by halogen, e.g., fluoromethyl and trifluoromethyl. The term “hydroxyl” means —OH.

The term “heteroatom” as used herein means an atom of any element other than carbon or hydrogen. Preferred heteroatoms are nitrogen, oxygen, sulfur and phosphorus.

The term “homeostasis” is art-recognized to mean maintenance of static, or constant, conditions in an internal environment.

The language “hormone secretion” is art-recognized and includes activities of vitamin D₃ compounds that control the transcription and processing responsible for secretion of a given hormone e.g., a parathyroid hormone (PTH) of a vitamin D₃ responsive cell (Bouillon, R. et al. (1995) Endocrine Reviews 16(2):235-237).

The language “hypercalcemia” or “hypercalcemic activity” is intended to have its accepted clinical meaning, namely, increases in calcium serum levels that are manifested in a subject by the following side effects, depression of central and peripheral nervous system, muscular weakness, constipation, abdominal pain, lack of appetite and, depressed relaxation of the heart during diastole. Symptomatic manifestations of hypercalcemia are triggered by a stimulation of at least one of the following activities, intestinal calcium transport, bone calcium metabolism and osteocalcin synthesis (reviewed in Boullion, R. et al. (1995) Endocrinology Reviews 16(2): 200-257).

The terms “hyperproliferative” and “neoplastic” are used interchangeably, and include those cells having the capacity for autonomous growth, i.e., an abnormal state or condition characterized by rapidly proliferating cell growth. Hyperproliferative and neoplastic disease states may be categorized as pathologic, i.e., characterizing or constituting a disease state, or may be categorized as non-pathologic, i.e., a deviation from normal but not associated with a disease state. The term is meant to include all types of cancerous growths or oncogenic processes, metastatic tissues or malignantly transformed cells, tissues, or organs, irrespective of histopathologic type or stage of invasiveness. “Pathologic hyperproliferative” cells occur in disease states characterized by malignant tumor growth. Examples of non-pathologic hyperproliferative cells include proliferation of cells associated with wound repair.

An “ILT3-associated disorder” includes a disease, disorder or condition which is associated with an ILT3 molecule. ILT3 associated disorders include disorders in which ILT3 activity is aberrant or in which a non-ILT3 activity that would benefit from modulation of an ILT3 activity is aberrant. In one embodiment, the ILT3-associated disorder is an immune disorder, e.g., an autoimmune disorder, such as type 1 insulin-dependent diabetes mellitus, adult respiratory distress syndrome, inflammatory bowel disease, dermatitis, meningitis, thrombotic thrombocytopenic purpura, Sjogren's syndrome, encephalitis, uveitic, leukocyte adhesion deficiency, rheumatoid arthritis, rheumatic fever, Reiter's syndrome, psoriatic arthritis, progressive systemic sclerosis, primary biliary cirrhosis, pemphigus, pemphigoid, necrotizing vasculitis, myasthenia gravis, multiple sclerosis, lupus erythematosus, polymyositis, sarcoidosis, granulomatosis, vasculitis, pernicious anemia, CNS inflammatory disorder, antigen-antibody complex mediated diseases, autoimmune haemolytic anemia, Hashimoto's thyroiditis, Graves disease, habitual spontaneous abortions, Reynard's syndrome, glomerulonephritis, dermatomyositis, chronic active hepatitis, celiac disease, autoimmune complications of AIDS, atrophic gastritis, ankylosing spondylitis and Addison's disease; or transplant rejection, such as GVHD. In certain embodiments of the invention, the ILT3 associated disorder is an immune disorders, such as transplant rejections, graft versus host disease and autoimmune disorders.

The language “immunoglobulin-like transcript 3” or “ILT3” refers to a cell surface molecule of the immunoglobulin superfamily, which is expressed by antigen-presenting cells (APCs) such as monocytes, macrophages and dendritic cells. ILT3 is a member of the immunoglobulin-like transcript (I) family and displays a long cytoplasmic tail containing putative immunoreceptor tyrosine-based inhibitory motifs (ITIMs). ILT3 has been shown to behave as an inhibitory receptor when cross-linked to a stimulatory receptor. A cytoplasmic component of the ILT3-mediated signaling pathway is the SH2-containing phosphatase SHP-1, which becomes associated with ILT3 upon cross-linking. ILT3 is also internalized and ILT3 ligands are efficiently presented to specific T cells (see, e.g., Cella, M. et al. (1997) J. Exp. Med. 185:1743). The determination of whether the candidate vitamin D₃ compound modulates the expression of the ILT3 surface molecule can be accomplished, for example, by comparison of ILT3 surface molecule expression to a control, by measuring mRNA expression, or by measuring protein expression.

The term “immune response” includes T and/or B cell responses, e.g., cellular and/or humoral immune responses. The claimed methods can be used to reduce both primary and secondary immune responses. The immune response of a subject can be determined by, for example, assaying antibody production, immune cell proliferation, the release of cytokines, the expression of cell surface markers, cytotoxicity, and the like.

The terms “immunological tolerance” or “tolerance to an antigen” or “immune tolerance” include unresponsiveness to an antigen without the induction of a prolonged generalized immune deficiency. Consequently, according to the invention, a tolerant host is capable of reacting to antigens other than the tolerizing antigen. Tolerance represents an induced depression in the response of a subject that, had it not been subjected to the tolerance-inducing procedure, would be competent to mount an immune response to that antigen. In one embodiment of the invention, immunological tolerance is induced in an antigen-presenting cell, e.g., an antigen-presenting cell derived from the myeloid or lymphoid lineage, dendritic cells, monocytes and macrophages.

The language “immunosuppressive activity” refers to the process of inhibiting a normal immune response. Included in this response is when T and/or B clones of lymphocytes are depleted in size or suppressed in their reactivity, expansion or differentiation. Immunosuppressive activity may be in the form of inhibiting or blocking an immune response already in progress or may involve preventing the induction of an immune response. The functions of activated T cells may be inhibited by suppressing immune cell responses or by inducing specific tolerance, or both. Immunosuppression of T cell responses is generally an active, non-antigen-specific, process that requires continuous exposure of the T cells to the suppressive agent. Tolerance, which involves inducing non-responsiveness or anergy in T cells, is distinguishable from immunosuppression in that it is generally antigen-specific and persists after exposure to the tolerizing agent has ceased. Operationally, tolerance can be demonstrated by the lack of a T cell response upon re-exposure to specific antigen in the absence of the tolerizing agent.

The language “improved biological properties” refers to any activity inherent in a compound of the invention that enhances its effectiveness in vivo. In a preferred embodiment, this term refers to any qualitative or quantitative improved therapeutic property of a vitamin D₃ compound, such as reduced toxicity, e.g., reduced hypercalcemic activity.

The language “inhibiting the growth” of the neoplasm includes the slowing, interrupting, arresting or stopping its growth and metastases and does not necessarily indicate a total elimination of the neoplastic growth.

The phrase “inhibition of an immune response” is intended to include decreases in T cell proliferation and activity, e.g., a decrease in IL₂, interferon-γ, GM-CSF synthesis and secretion (Lemire, J. M. (1992) J. Cell Biochemistry 49:26-31, Lemire, J. M. et al. (1994) Endocrinology 135 (6): 2813-2821; Bouillon, R. et al. (1995) Endocrine Review 16 (2):231-32).

The term “isomers” or “stereoisomers” refers to compounds which have identical chemical constitution, but differ with regard to the arrangement of the atoms or groups in space.

The term “leukemia” is intended to have its clinical meaning, namely, a neoplastic disease in which white corpuscle maturation is arrested at a primitive stage of cell development. The disease is characterized by an increased number of leukemic blast cells in the bone marrow, and by varying degrees of failure to produce normal hematopoietic cells. The condition may be either acute or chronic. Leukemias are further typically categorized as being either lymphocytic i.e., being characterized by cells which have properties in common with normal lymphocytes, or myelocytic (or myelogenous), i.e., characterized by cells having some characteristics of normal granulocytic cells. Acute lymphocytic leukemia (“ALL”) arises in lymphoid tissue, and ordinarily first manifests its presence in bone marrow. Acute myelocytic leukemia (“AML”) arises from bone marrow hematopoietic stem cells or their progeny. The term acute myelocytic leukemia subsumes several subtypes of leukemia: myeloblastic leukemia, promyelocytic leukemia, and myelomonocytic leukemia. In addition, leukemias with erythroid or megakaryocytic properties are considered myelogenous leukemias as well.

The term “leukemic cancer” refers to all cancers or neoplasias of the hemopoietic and immune systems (blood and lymphatic system). The acute and chronic leukemias, together with the other types of tumors of the blood, bone marrow cells (myelomas), and lymph tissue (lymphomas), cause about 10% of all cancer deaths and about 50% of all cancer deaths in children and adults less than 30 years old. Chronic myelogenous leukemia (CML), also known as chronic granulocytic leukemia (CGL), is a neoplastic disorder of the hematopoietic stem cell. The term “leukemia” is art recognized and refers to a progressive, malignant disease of the blood-forming organs, marked by distorted proliferation and development of leukocytes and their precursors in the blood and bone marrow.

The term “modulate” refers to increases or decreases in the activity of a cell in response to exposure to a compound of the invention, e.g., the inhibition of proliferation and/or induction of differentiation of at least a sub-population of cells in an animal such that a desired end result is achieved, e.g., a therapeutic result. In preferred embodiments, this phrase is intended to include hyperactive conditions that result in pathological disorders.

The common medical meaning of the term “neoplasia” refers to “new cell growth” that results as a loss of responsiveness to normal growth controls, e.g. to neoplastic cell growth. A “hyperplasia” refers to cells undergoing an abnormally high rate of growth. However, as used herein, the terms neoplasia and hyperplasia can be used interchangably, as their context will reveal, referring to generally to cells experiencing abnormal cell growth rates. Neoplasias and hyperplasias include “tumors,” which may be either benign, premalignant or malignant.

The language “non-genomic” vitamin D₃ activities include cellular (e.g., calcium transport across a tissue) and subcellular activities (e.g., membrane calcium transport opening of voltage-gated calcium channels, changes in intracellular second messengers) elicited by vitamin D₃ compounds in a responsive cell. Electrophysiological and biochemical techniques for detecting these activities are known in the art. An example of a particular well-studied non-genomic activity is the rapid hormonal stimulation of intestinal calcium mobilization, termed “transcaltachia” (Nemere I. et al. (1984) Endocrinology 115:1476-1483; Lieberherr M. et al. (1989) J. Biol. Chem. 264:20403-20406; Wali R. K. et al. (1992) Endocrinology 131:1125-1133; Wali R. K. et al. (1992) Am. J. Physiol. 262:G945-G953; Wali R. K. et al. (1990) J. Clin. Invest. 85:1296-1303; Bolt M. J. G. et al. (1993) Biochem. J. 292:271-276). Detailed descriptions of experimental transcaltachia are provided in Norman, A. W. (1993) Endocrinology 268(27):20022-20030; Yoshimoto, Y. and Norman, A. W. (1986) Endocrinology 118:2300-2304. Changes in calcium activity and second messenger systems are well known in the art and are extensively reviewed in Bouillion, R. et al. (1995) Endocrinology Review 16(2): 200-257; the description of which is incorporated herein by reference.

The phrases “parenteral administration” and “administered parenterally” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticulare, subcapsular, subarachnoid, intraspinal and intrasternal injection and infusion.

The terms “polycyclyl” or “polycyclic radical” refer to the radical of two or more cyclic rings (e.g., cycloalkyls, cycloalkenyls, cycloalkynyls, aryls and/or heterocyclyls) in which two or more carbons are common to two adjoining rings, e.g., the rings are “fused rings”. Rings that are joined through non-adjacent atoms are termed “bridged” rings. Each of the rings of the polycycle can be substituted with such substituents as described above, as for example, halogen, hydroxyl, alkylcarbonyloxy, arylcarbonyloxy, alkoxycarbonyloxy, aryloxycarbonyloxy, carboxylate, alkylcarbonyl, alkoxycarbonyl, aminocarbonyl, alkylthiocarbonyl, alkoxyl, phosphate, phosphonato, phosphinato, cyano, amino (including alkyl amino, dialkylamino, arylamino, diarylamino, and alkylarylamino), acylamino (including alkylcarbonylamino, arylcarbonylamino, carbamoyl and ureido), amidino, imino, sulfhydryl, alkylthio, arylthio, thiocarboxylate, sulfates, sulfonato, sulfamoyl, sulfonamido, nitro, trifluoromethyl, cyano, azido, heterocyclyl, alkyl, alkylaryl, or an aromatic or heteroaromatic moiety.

The term “prodrug” includes compounds with moieties which can be metabolized in vivo. Generally, the prodrugs are metabolized in vivo by esterases or by other mechanisms to active drugs. Examples of prodrugs and their uses are well known in the art (See, e.g., Berge et al. (1977) “Pharmaceutical Salts”, J. Pharm. Sci. 66:1-19). The prodrugs can be prepared in situ during the final isolation and purification of the compounds, or by separately reacting the purified compound in its free acid form or hydroxyl with a suitable esterifying agent Hydroxyl groups can be converted into esters via treatment with a carboxylic acid. Examples of prodrug moieties include substituted and unsubstituted, branch or unbranched lower alkyl ester moieties, (e.g., propionoic acid esters), lower alkenyl esters, di-lower allyl-amino lower-alkyl esters (e.g., dimethylaminoethyl ester), acylamino lower alkyl esters (e.g., acetyloxymethyl ester), acyloxy lower alkyl esters (e.g., pivaloyloxymethyl ester), aryl esters (phenyl ester), aryl-lower alkyl esters (e.g., benzyl ester), substituted (e.g., with methyl, halo, or methoxy substituents) aryl and aryl-lower alkyl esters, amides, lower-alkyl amides, di-lower alkyl amides, and hydroxy amides. Preferred prodrug moieties are propionoic acid esters and acyl esters. Prodrugs which are converted to active forms through other mechanisms in vivo are also included.

The language “a prophylactically effective anti-neoplastic amount” of a compound refers to an amount of a vitamin D₃ compound of the formula (I) or otherwise described herein which is effective, upon single or multiple dose administration to the patient, in preventing or delaying the occurrence of the onset of a neoplastic disease state.

The term “psoriasis” is intended to have its medical meaning, namely, a disease which afflicts primarily the skin and produces raised, thickened, scaling, nonscarring lesions. The lesions are usually sharply demarcated erythematous papules covered with overlapping shiny scales. The scales are typically silvery or slightly opalescent. Involvement of the nails frequently occurs resulting in pitting, separation of the nail, thickening and discoloration. Psoriasis is sometimes associated with arthritis, and it may be crippling.

The language “reduced toxicity” is intended to include a reduction in any undesired side effect elicited by a vitamin D₃ compound when administered in vivo, e.g., a reduction in the hypercalcemic activity.

The term “sarcoma” is art recognized and refers to malignant tumors of mesenchymal derivation.

The term “secosteroid” is art-recognized and includes compounds in which one of the cyclopentanoperhydro-phenanthrene rings of the steroid ring structure is broken. 1α,25(OH)₂D₃ and analogs thereof are hormonally active secosteroids. In the case of vitamin D₃, the 9-10 carbon-carbon bond of the B-ring is broken, generating a seco-B-steroid. The official IUPAC name for vitamin D₃ is 9,10-secocholesta-5,7,10(19)-trien-3B-ol. For convenience, a 6-s-trans conformer of 1α,25(OH)₂D₃ is illustrated herein having all carbon atoms numbered using standard steroid notation.

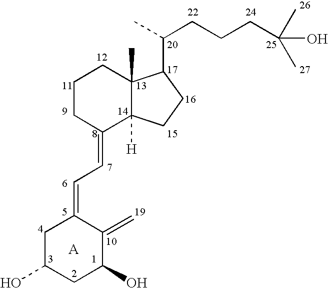

In the formulas presented herein, the various substituents on ring A are illustrated as joined to the steroid nucleus by one of these notations: a dotted line ( [symbol] ) indicating a substituent which is in the β-orientation (i.e., above the plane of the ring), a wedged solid line ( [symbol] ) indicating a substituent which is in the α-orientation (i.e., below the plane of the molecule), or a wavy line

[symbol]

indicating that a substituent may be either above or below the plane of the ring. In regard to ring A, it should be understood that the stereochemical convention in the vitamin D field is opposite from the general chemical field, wherein a dotted line indicates a substituent on Ring A which is in an α-orientation (i.e., below the plane of the molecule), and a wedged solid line indicates a substituent on ring A which is in the β-orientation (i.e., above the plane of the ring). As shown, the A ring of the hormone 1α,25(OH)₂D₃ contains two asymmetric centers at carbons 1 and 3, each one containing a hydroxyl group in well-characterized configurations, namely the 1α- and 3β-hydroxyl groups. In other words, carbons 1 and 3 of the A ring are said to be “chiral carbons” or “carbon centers.”

The term “sulfhydryl” or “thiol” means —SH.

The term “subject” includes organisms which are capable of suffering from a vitamin D₃ associated state or who could otherwise benefit from the administration of a vitamin D₃ compound of the invention, such as human and non-human animals. Preferred human animals include human patients suffering from or prone to suffering from a vitamin D₃ associated state, as described herein. The term “non-human animals” of the invention includes all vertebrates, e.g., mammals, e.g., rodents, e.g., mice, and non-mammals, such as non-human primates, sheep, dog, cow, chickens, amphibians, reptiles, etc.

The phrases “systemic administration,” “administered systemically”, “peripheral administration” and “administered peripherally” as used herein mean the administration of a vitamin D₃ compound(s), drug or other material, such that it enters the patient's system and, thus, is subject to metabolism and other like processes, for example, subcutaneous administration.

The language “therapeutically effective anti-neoplastic amount” of a vitamin D₃ compound of the invention refers to an amount of an agent which is effective, upon single or multiple dose administration to the patient, in inhibiting the growth of a neoplastic vitamin D₃-responsive cells, or in prolonging the survivability of the patient with such neoplastic cells beyond that expected in the absence of such treatment.

The language “transplant rejection” refers to an immune reaction directed against a transplanted organ(s) from other human donors (allografts) or from other species such as sheep, pigs, or non-human primates (xenografts). Therefore, the method of the invention is useful for preventing an immune reaction to transplanted organs from other human donors (allografts) or from other species (xenografts). Such tissues for transplantation include, but are not limited to, heart, liver, kidney, lung, pancreas, pancreatic islets, bone marrow, brain tissue, cornea, bone, intestine, skin, and hematopoietic cells. Also included within this definition is “graft versus host disease” of “GVHD,” which is a condition where the graft cells mount an immune response against the host. Therefore, the method of the invention is useful in preventing graft versus host disease in cases of mismatched bone marrow or lymphoid tissue transplanted for the treatment of acute leukemia, aplastic anemia, and enzyme or immune deficiencies, for example. The term “transplant rejection” also includes disease symptoms characterized by loss of organ function. For example, kidney rejection would be characterized by a rising creatine level in blood. Heart rejection is characterized by an endomyocardial biopsy, while pancreas rejection is characterized by rising blood glucose levels. Liver rejection is characterized by the levels of transaminases of liver origin and bilirubin levels in blood. Intestine rejection is determined by biopsy, while lung rejection is determined by measurement of blood oxygenation.

The terms “urogenital”, “urogenital system” and “urogential tract” are used interchangeably and are intended to include all organs involved in reproduction and in the formation and voidance of urine. Included with in these terms are the kidneys, bladder and prostate.

The term “VDR” is intended to include members of the type II class of steroid/thyroid superfamily of receptors (Stunnenberg, H. G. (1993) Bio Essays 15(5):309-15), which are able to bind and transactivate through the vitamin D response element (VDRE) in the absence of a ligand (Damm et al. (1989) Nature 339:593-97; Sap et al. Nature 343:177-180).

The term “VDRE” refers to DNA sequences composed of half-sites arranged as direct repeats. It is known in the art that type II receptors do not bind to their respective binding site as homodimers but require an auxiliary factor, RXR (e.g. RXRα, RXRβ, RXRγ) for high affinity binding Yu et al. (1991) Cell 67:1251-1266; Bugge et al. (1992) EMBO J. 11:1409-1418; Kliewer et al. (1992) Nature 355:446-449; Leid et al. (1992) EMBO J. 11:1419-1435; Zhang et al. (1992) Nature 355:441-446).

The language “vitamin D₃ associated state” is a state which can be prevented, treated or otherwise ameliorated by administration of one or more compounds of the invention. Vitamin D₃ associated states include ILT3-associated disorders, disorders characterized by an aberrant activity of a vitamin D₃-responsive cell, disorders characterized by a deregulation of calcium and phosphate metabolism, and other disorders or states described herein.

The term “vitamin D₃-responsive cell” includes any cell which is capable of responding to a vitamin D₃ compound having the formula I or otherwise described herein, or is associated with disorders involving an aberrant activity of hyperproliferative skin cells, parathyroid cells, neoplastic cells, immune cells, and bone cells. These cells can respond to vitamin D₃ activation by triggering genomic and/or non-genomic responses that ultimately result in the modulation of cell proliferation, differentiation survival, and/or other cellular activities such as hormone secretion. In a preferred embodiment, the ultimate responses of a cell are inhibition of cell proliferation and/or induction of differentiation-specific genes. Exemplary vitamin D₃ responsive cells include immune cells, bone cells, neuronal cells, endocrine cells, neoplastic cells, epidermal cells, endodermal cells, smooth muscle cells, among others.

With respect to the nomenclature of a chiral center, terms “d” and “l” configuration are as defined by the IUPAC Recommendations. As to the use of the terms, diastereomer, racemate, epimer and enantiomer will be used in their normal context to describe the stereochemistry of preparations.

### 2. Gemini Vitamin D₃ Compounds

In the structure of 1,25-dihydroxy vitamin D₃ gemini analogs, two fall side chains are attached at the C-20 position. Gemini compounds exert a full spectrum of 1,25(OH)₂D₃ biological activities such as binding to the specific nuclear receptor VDR, suppression of the increased parathyroid hormone levels in 5,6-nephrectomized rats, suppression of INF-γ release in MLR cells, stimulation of EL-60 leukemia cell differentiation and inhibition of solid tumor cell proliferation (Uskokovic, M. R et al., “Synthesis and preliminary evaluation of the biological properties of a 1α,25-dihydroxyvitamin D₃ analogue with two side-chains.” Vitamin D: Chemistry, Biology and Clinical Applications of the Steroid Hormone; Norman, A. W., et al., Eds.; University of California: Riverside, 1997; pp 19-21; Norman et al., J. Med. Chem. 2000, Vol. 43, 2719-2730).

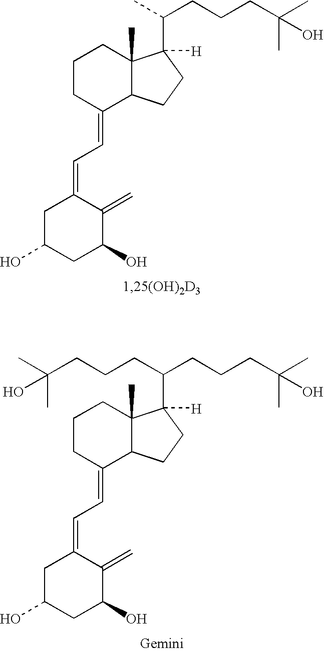

Both in vivo and in cellular cultures, 1,25-(OH)₂D₃ undergoes a cascade of metabolic modifications initiated by the influence of 24R-hydroxylase enzyme. First 24R-hydroxy metabolite is formed, which is oxydized to 24-keto intermediate, and then 23S-hydroxylation and fragmentation produce the fully inactive calcitroic acid.

Unexpectedly, metabolism of gemini in bone cells produces a single 24R-hydroxy metabolite, without affecting the other side chain. There are two possible structures for this metabolite that differ in the configuration at C-20, 20R and 20S.

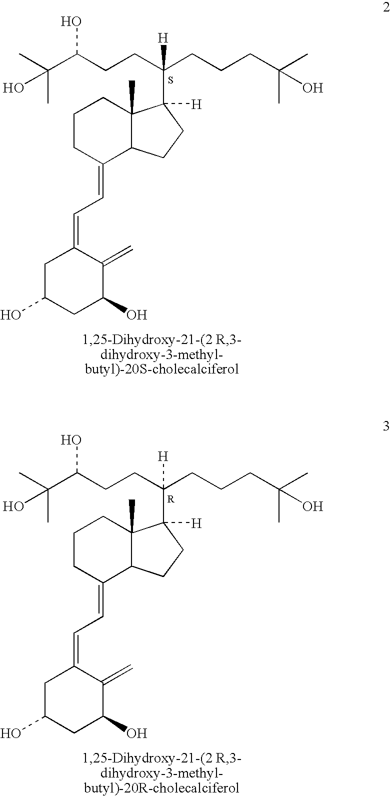

These two 20-epimeric-24R-hydroxy gemini analogs have been prepared by stereoselective synthesis and tested in comparison to 1,25(OH)₂D₃ and gemini for VDR-binding, HL-60 cell differentiation, maximum tolerated dose in mice, inhibition of INF-γ release in MLR, rennin suppression, antiproliferative effect in cancer bladder assay, etc. (See Examples 1-14 below.)

Thus, in one aspect, the invention provides Gemini vitamin D₃ compounds of formula I:

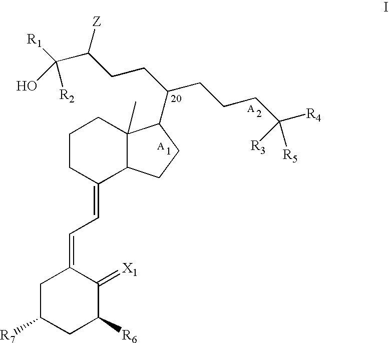

wherein:

A₁ is a single or double bond;

A₂ is a single, a double or a triple bond;

R₁, R₂, R₃ and P4 are each independently C₁-C₄ alkyl, C₁-C₄ deuteroalkyl, hydroxyalkyl, or haloalkyl;

R₅, R₆ and R₇ are each independently hydroxyl, OC(O)C₁-C₄ alkyl, OC(O)hydroxyalkyl, or OC(O)haloalkyl;

the configuration at C₂₀ is R or S;

X₁ is H₂ or CH₂;

Z is hydrogen when at least one of R₁ and R₂ is C₁-C₄ deuteroalkyl and at least one of R₃ and R₄ is haloalkyl or when at least one of R₁ and R₂ is haloalkyl and at least one of R₃ and R₄ is C₁-C₄ deuteroalkyl; or Z is —OH, ═O, —SH, or —NH₂;

and pharmaceutically acceptable esters, salts, and prodrugs thereof.

Various embodiments of this aspect of the invention include individual compounds of formula I wherein: A₁ is a single bond; A₂ is a single bond; A₂ is a triple bond; R₁, R₂, R₃, and R₄ are each independently methyl or ethyl; R₁, R₂, R₃, and R₄ are each independently C₁-C₄ deuteroalkyl or haloalkyl; R₅ is hydroxyl; R₆ and R₇ are hydroxyl; R₆ and R₇ are each OC(O)C₁-C₄ alkyl; X₁ I is H₂; X₁ I is CH₂; Z is hydrogen; or Z is ═O.

In certain embodiments, R₅, R₆ and R₇ are hydroxyl. In other embodiments, R₆ and R₇ are each acetyloxy.

In yet other embodiments, Z is hydrogen when at least one of R₁ and R₂ is C₁-C₄ deuteroalkyl and at least one of R₃ and R₄ is haloalkyl or when at least one of R₁ and R₂ is haloalkyl and at least one of R₃ and R₄ is C₁-C₄ deuteroalkyl; Z is —OH, ═O, —SH, or —NH₂ when X₁ is CH₂; Z is —OH, ═O, —SH, or —NH₂ when X₁ is H₂ and the configuration at C₂₀ is S; or Z is ═O, —SH, or —NH₂ when X₁ I is H₂ and the configuration at C₂₀ is R. In one embodiment, Z is —OH.

Still other embodiments of this aspect of invention include those wherein X₁ I is CH₂; A₂ is a single bond; R₁, R₂, R₃, and R₄ are each independently methyl or ethyl; and Z is —OH. In one embodiment, X₁ I is CH₂; A₂ is a single bond; R₁, R₂, R₃, and R₄ are each independently methyl or ethyl; and Z is ═O. In one embodiment, X₁ I is H₂; A₂ is a single bond; R₁, R₂, R₃, and R₄ are each independently methyl or ethyl; the configuration at C₂₀ is S; and Z is OH. In another embodiment, X₁ is H₂; A₂ is a single bond; R₁, R₂, R₃, and P4 are each independently methyl or ethyl; and Z is ═O. In these embodiments, R₁, R₂, R₃, and R₄ are advantageously each methyl.

In certain embodiments, the haloalkyl is fluoroalkyl. Advantageously, fluoroalkyl is fluoromethyl or trifluoromethyl.

Additional embodiments of this aspect of the invention include compounds X₁ I is H₂; A₂ is a triple bond; R₁ and R₂ are each C₁-C₄ deuteroalkyl; R₃ and R₄ are each haloalkyl; and Z is hydrogen. In other embodiments, X₁ I is CH₂; A₂ is a triple bond; R₁ and R₂ are each C₁-C₄ deuteroalkyl; R₃ and R₄ are each haloalkyl; and Z is hydrogen. In these embodiments, R₁ and R₂ are advantageously each deuteromethyl and R₃ and R₄ are advantageously each trifluoromethyl.

Specific compounds of the invention include: 1,25-Dihydroxy-21-(2R,3-dihydroxy-3-methyl-butyl)-20R-cholecalciferol:

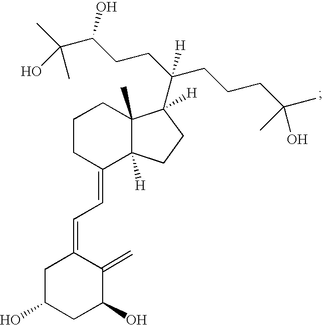

1,25-Dihydroxy-21-(2R,3-dihydroxy-3-methyl-butyl)-20S-cholecalciferol:

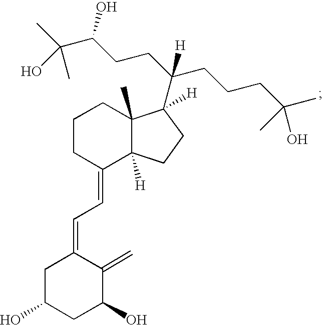

1,25-Dihydroxy-21-(2R,3-dihydroxy-3-methyl-butyl)-20S-19-nor-cholecalciferol:

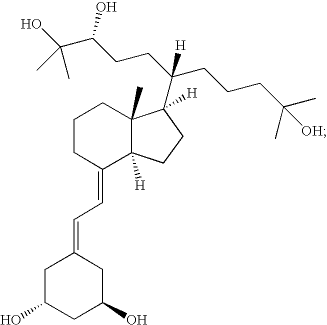

1,25-Dihydroxy-20S-21-(3-hydroxy-3-methyl-butyl)-24-keto-19-nor-cholecalciferol:

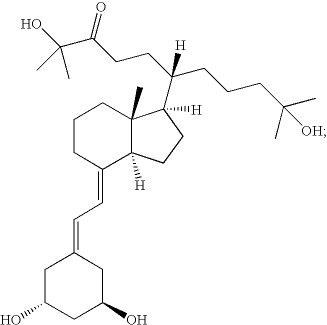

1,25-Dihydroxy-20S-21-(3-hydroxy-3-methyl-butyl)-24-keto-cholecalciferol:

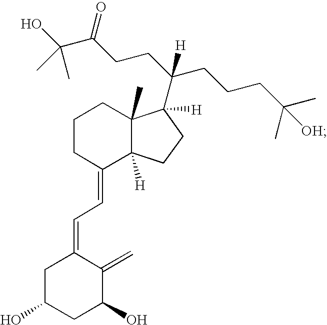

1,25-Dihydroxy-21(3-hydroxy-3-trifluoromethyl-4-trifluoro-butynyl)-26,27-hexadeutero-19-nor-20S-cholecalciferol:

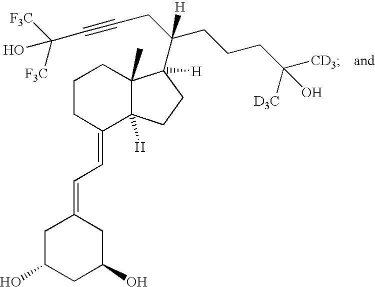

1,25-Dihydroxy-21-(3-hydroxy-3-trifluoromethyl-4-trifluoro-butynyl)-26,27-hexadeutero-20S-cholecalciferol:

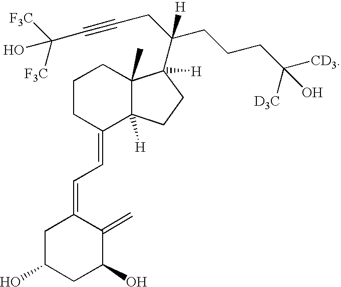

The structures of some of the compounds of the invention include asymmetric carbon atoms. Accordingly, the isomers arising from such asymmetry (e.g., all enantiomers and diastereomers) are included within the scope of this invention, unless indicated otherwise. Such isomers can be obtained in substantially pure form by classical separation techniques and/or by stereochemically controlled synthesis.

Naturally occurring or synthetic isomers can be separated in several ways known in the art. Methods for separating a racemic mixture of two enantiomers include chromatography using a chiral stationary phase (see, e.g., “Chiral Liquid Chromatography,” W. J. Lough, Ed. Chapman and Hall, New York (1989)). Enantiomers can also be separated by classical resolution techniques. For example, formation of diastereomeric salts and fractional crystallization can be used to separate enantiomers. For the separation of enantiomers of carboxylic acids, the diastereomeric salts can be formed by addition of enantiomerically pure chiral bases such as brucine, quinine, ephedrine, strychnine, and the like. Alternatively, diastereomeric esters can be formed with enantiomerically pure chiral alcohols such as menthol, followed by separation of the diastereomeric esters and hydrolysis to yield the free, enantiomerically enriched carboxylic acid. For separation of the optical isomers of amino compounds, addition of chiral carboxylic or sulfonic acids, such as camphorsulfonic acid, tartaric acid, mandelic acid, or lactic acid can result in formation of the diastereomeric salts.

### 3. Uses of the Vitamin D₃ Compounds of the Invention

In another aspect, the invention also provides methods for treating a subject for a vitamin D₃ associated state, by administering to the subject an effective amount of a vitamin D₃ compound of formula (I) or otherwise described herein. Vitamin D₃ associated states include disorders involving an aberrant activity of a vitamin D₃-responsive cell, e.g., neoplastic cells, hyperproliferative skin cells, parathyroid cells, immune cells and bone cells, among others. Vitamin D₃ associated states also include ILT3-associated disorders.

In current methods, the use of vitamin D₃ compounds has been limited because of their hypercalcemic effects. The Gemini vitamin D₃ compounds of the invention can provide a less toxic alternative to current methods of treatment.

In certain embodiments, the subject is a mammal, in particular a human.

In accordance with the methods of the invention, the Gemini vitamin D₃ compound can be administered in combination with a pharmaceutically acceptable carrier. In advantageous embodiments, the pharmaceutically-acceptable carrier provides sustained delivery of the Gemini vitamin D₃ compound to a subject for at least four weeks after administration to the subject.

In certain embodiments, the Gemini vitamin D₃ compound is administered orally. In other embodiments, the vitamin D₃ compound is administered intravenously. In yet other embodiments, the vitamin D₃ compound is administered topically. In still other embodiments, the vitamin D₃ compound is administered topically is administered parenterally.

Although dosages may vary depending on the particular indication, route of administration and subject, the Gemini vitamin D₃ compounds are administered at a concentration of about 0.001 μg to about 100 μg/kg of body weight.

A. Hyperproliferative Conditions

In another aspect, the present invention provides a method of treating a subject for a disorder characterized by aberrant activity of a vitamin D₃-responsive cell. The method involves administering to the subject an effective amount of a pharmaceutical composition of a vitamin D₃ compound of formula I or otherwise described herein such that the activity of the cell is modulated.

In certain embodiments, the cells to be treated are hyperproliferative cells. As described in greater detail below, the vitamin D₃ compounds of the invention can be used to inhibit the proliferation of a variety of hyperplastic and neoplastic tissues. In accordance with the present invention, vitamin D₃ compounds of the invention can be used in the treatment of both pathologic and non-pathologic proliferative conditions characterized by unwanted growth of vitamin D₃-responsive cells, e.g., hyperproliferative skin cells, immune cells, and tissue having transformed cells, e.g., such as carcinomas, sarcomas and leukemias. In other embodiments, the cells to be treated are aberrant secretory cells, e.g., parathyroid cells, immune cells.

In one embodiment, this invention features a method for inhibiting the proliferation and/or inducing the differentiation of a hyperproliferative skin cell, e.g., an epidermal or an epithelial cell, e.g. a keratinocytes, by contacting the cells with a vitamin D₃ compound of the invention. In general, the method includes a step of contacting a pathological or non-pathological hyperproliferative cell with an effective amount of such vitamin D₃ compound to promote the differentiation of the hyperproliferative cells The present method can be performed on cells in culture, e.g. in vitro or ex vivo, or can be performed on cells present in an animal subject, e.g., as part of an in vivo therapeutic protocol. The therapeutic regimen can be carried out on a human or any other animal subject.

The vitamin D₃ compounds of the present invention can be used to treat a hyperproliferative skin disorder. Exemplary disorders include, but are not limited to, psoriasis, basal cell carcinoma, keratinization disorders and keratosis. Additional examples of these disorders include eczema; lupus associated skin lesions; psoriatic arthritis; rheumatoid arthritis that involves hyperproliferation and inflammation of epithelial-related cells lining the joint capsule; dermatitides such as seborrheic dermatitis and solar dermatitis; keratoses such as seborrheic keratosis, senile keratosis, actinic keratosis. photo-induced keratosis, and keratosis follicularis; acne vulgaris; keloids and prophylaxis against keloid formation; nevi; warts including verruca, condyloma or condyloma acuminatum, and human papilloma viral (BPV) infections such as venereal warts; leukoplakia; lichen planus; and keratitis.

In an illustrative example, vitamin D₃ compounds of the invention can be used to inhibit the hyperproliferation of keratinocytes in treating diseases such as psoriasis by administering an effective amount of these compounds to a subject in need of treatment. The term “psoriasis” is intended to have its medical meaning, namely, a disease which afflicts primarily the skin and produces raised, thickened, scaling, nonscarring lesions. The lesions are usually sharply demarcated erythematous papules covered with overlapping shiny scales. The scales are typically silvery or slightly opalescent. Involvement of the nails frequently occurs resulting in pitting, separation of the nail, thickening and discoloration. Psoriasis is sometimes associated with arthritis, and it may be crippling. Hyperproliferation of keratinocytes is a key feature of psoriatic epidermal hyperplasia along with epidermal inflammation and reduced differentiation of keratinocytes. Multiple mechanisms have been invoked to explain the keratinocyte hyperproliferation that characterizes psoriasis. Disordered cellular immunity has also been implicated in the pathogenesis of psoriasis.

B. Neoplasia

The invention also features methods for inhibiting the proliferation and/or reversing the transformed phenotype of vitamin D₃-responsive hyperproliferative cells by contacting the cells with a vitamin D₃ compound of formula (a) or otherwise described herein. In general, the method includes a step of contacting pathological or non-pathological hyperproliferative cells with an effective amount of a vitamin D₃ compound of the invention for promoting the differentiation of the hyperproliferative cells. The present method can be performed on cells in culture, e.g., in vitro or ex vivo, or can be performed on cells present in an animal subject, e.g., as part of an in vivo therapeutic protocol. The therapeutic regimen can be carried out on a human or other subject.

The vitamin D₃ compounds of formula I or otherwise described herein can be tested initially in vitro for their inhibitory effects in the proliferation of neoplastic cells. Examples of cell lines that can be used are transformed cells, e.g., the human promyeloid leukemia cell line HL-60, and the human myeloid leukemia U-937 cell line (Abe E. et al. (1981) Proc. Natl. Acad. Sci. USA 78:4990-4994; Song L. N. and Cheng T. (1992) Biochem Pharmacol 43:2292-2295; Zhou J. Y. et al. (1989) Blood 74:82-93; U.S. Pat. No. 5,401,733, U.S. Pat. No. 5,087,619). Alternatively, the antitumoral effects of vitamin D₃ compounds of the invention can be tested in vivo using various animal models known in the art and summarized in Bouillon, R. et al. (1995) Endocrine Reviews 16(2):233 (Table E), which is incorporated by reference herein. For example, SL mice are routinely used in the art to test vitamin D₃ compounds of the invention as models for MI myeloid leukemia (Honma et al. (1983) Cell Biol. 80:201-204; Kasukabe T. et al. (1987) Cancer Res. 47:567-572); breast cancer studies can-be performed in, for example, nude mice models for human MX1 (ER) (Abe S. et al. (1991) Endocrinology 129:832-837; other cancers, e.g., colon cancer, melanoma osteosarcoma, can be characterized in, for example, nude mice models as describe in (Eisman J. A. et al. (1987) Cancer Res. 47:21-25; Kawaura A. et al. (1990) Cancer Lett 55:149-152; Belleli A. (1992) Carcinogenesis 13:2293-2298; Tsuchiya H. et al. (1993) J. Orthopaed Res. 11:122-130).

The subject method may also be used to inhibit the proliferation of hyperplastic/neoplastic cells of hematopoietic origin, e.g., arising from myeloid, lymphoid or erythroid lineages, or precursor cells thereof. For instance, the present invention contemplates the treatment of various myeloid disorders including, but not limited to, acute promyeloid leukemia (APML), acute myelogenous leukemia (AML) and chronic myelogenous leukemia (CML) (reviewed in Vaickus, L. (1991) Crit. Rev. in Oncol./Hemotol. 11:267-97). Lymphoid malignancies which may be treated by the subject method include, but are not limited to acute lymphoblastic leukemia (ALL) which includes B-lineage ALL and T-lineage ALL, chronic lymphocytic leukemia (CLL), prolymphocytic leukemia (PLL), hairy cell leukemia (BLL) and Waldenstrom's macroglobulinemia (WM). Additional forms of malignant lymphomas contemplated by the treatment method of the present invention include, but are not limited to non-Hodgkin lymphoma and variants thereof, peripheral T cell lymphomas, adult T cell leukemia/lymphoma (ATL), cutaneous T-cell lymphoma (CTCL), large granular lymphocytic leukemia (LGF) and Hodgkin's disease.

In certain embodiments, the vitamin D₃ compounds of the invention can be used in combinatorial therapy with conventional cancer chemotherapeutics. Conventional treatment regimens for leukemia and for other tumors include radiation, drugs, or a combination of both. In addition to radiation, the following drugs, usually in combinations with each other, are often used to treat acute leukemias: vincristine, prednisone, methotrexate, mercaptopurine, cyclophosphamide, and cytarabine. In chronic leukemia, for example, busulfan, melphalan, and chlorambucil can be used in combination. AU of the conventional anti-cancer drugs are highly toxic and tend to make patients quite ill while undergoing treatment. Vigorous therapy is based on the premise that unless every leukemic cell is destroyed, the residual cells will multiply and cause a relapse.

The subject method can also be useful in treating malignancies of the various organ systems, such as affecting lung, breast, lymphoid, gastrointestinal, and urogenital tract as well as adenocarcinomas which include malignancies such as most colon cancers, renal-cell carcinoma, prostate cancer and/or testicular tumors, bladder cancer, non-small cell carcinoma of the lung, cancer of the small intestine and cancer of the esophagus.

According to the general paradigm of vitamin D₃ involvement in differentiation of transformed cells, exemplary solid tumors that can be treated according to the method of the present invention include vitamin D₃-responsive phenotypes of sarcomas and carcinomas such as, but not limited to: fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, pancreatic cancer, breast cancer, ovarian cancer, prostate cancer, bladder cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilms' tumor, cervical cancer, testicular tumor, lung carcinoma, small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, melanoma, neuroblastoma, and retinoblastoma.

Determination of a therapeutically effective anti-neoplastic amount or a prophylactically effective anti-neoplastic amount of the vitamin D₃ compound of the invention, can be readily made by the physician or veterinarian (the “attending clinician”), as one skilled in the art, by the use of known techniques and by observing results obtained under analogous circumstances. The dosages may be varied depending upon the requirements of the patient in the judgment of the attending clinician, the severity of the condition being treated and the particular compound being employed. In determining the therapeutically effective antineoplastic amount or dose, and the prophylactically effective antineoplastic amount or dose, a number of factors are considered by the attending clinician, including, but not limited to: the specific hyperplastic/neoplastic cell involved; pharmacodynamic characteristics of the particular agent and its mode and route of administration; the desirder time course of treatment; the species of mammal; its size, age, and general health; the specific disease involved; the degree of or involvement or the severity of the disease; the response of the individual patient; the particular compound administered; the mode of administration; the bioavailability characteristics of the preparation administered; the dose regimen selected; the kind of concurrent treatment (i.e., the interaction of the vitamin D₃ compounds of the invention with other co-administered therapeutics); and other relevant circumstances. U.S. Pat. No. 5,427,916, for example, describes method for predicting the effectiveness of antineoplastic therapy in individual patients, and illustrates certain methods which can be used in conjunction with the treatment protocols of the instant invention.

Treatment can be initiated with smaller dosages which are less than the optimum dose of the compound. Thereafter, the dosage should be increased by small increments until the optimum effect under the circumstances is reached. For convenience, the total daily dosage may be divided and administered in portions during the day if desired. A therapeutically effective antineoplastic amount and a prophylactically effective anti-neoplastic amount of a vitamin D₃ compound of the invention is expected to vary from about 0.1 milligram per kilogram of body weight per day (mg/kg/day) to about 100 mg/kg/day.

Compounds which are determined to be effective for the prevention or treatment of tumors in animals, e.g., dogs, rodents, may also be useful in treatment of tumors in humans. Those skilled in the art of treating tumor in humans will know, based upon the data obtained in animal studies, the dosage and route of administration of the compound to humans. In general, the dosage and route of administration in humans is expected to be similar to that in animals.

The identification of those patients who are in need of prophylactic treatment for hyperplastic/neoplastic disease states is well within the ability and knowledge of one skilled in the art. Certain of the methods for identification of patients which are at risk of developing neoplastic disease states which can be treated by the subject method are appreciated in the medical arts, such as family history of the development of a particular disease state and the presence of risk factors associated with the development of that disease state in the subject patient. A clinician skilled in the art can readily identify such candidate patients, by the use of, for example, clinical tests, physical examination and medical/family history.

C. Immunological Activity

Healthy individuals protect themselves against foreign invaders using many different mechanisms, including physical barriers, phagocytic cells in the blood and tissues, a class of immune cells known as lymphocytes, and various blood-born molecules. All of these mechanisms participate in defending individuals from a potentially hostile environment. Some of these defense mechanisms, known as natural or innate immunity, are present in an individual prior to exposure to infectious microbes or other foreign macromolecules, are not enhanced by such exposures, and do not discriminate among most foreign substances. Other defense mechanisms, known as acquired or specific immunity, are induced or stimulated by exposure of foreign substances, are exquisitely specific for distinct macromolecules, and increase in magnitude and defensive capabilities with each successive exposure to a particular macromolecule. Substances that induce a specific immune response are known as antigens (see, e.g., Abbas, A. et al., Cellular and Molecular Immunology, W.B. Saunders Company, Philadelphia, 1991; Silverstein, A. M. A history of Immunology, San Diego, Academic Press, 1989; Unanue A. et al., Textbook of Immunology, 2^{nd} ed. Williams and Wilkens, Baltimore, 1984).

One of the most remarkable properties of the immune system is its ability to distinguish between foreign antigens and self-antigens. Therefore, the lymphocytes in each individual are able to recognize and respond to many foreign antigens but are normally unresponsive to the potentially antigenic substances present in the individual. This immunological unresponsiveness is referred to as immune tolerance (see, e.g., Burt R K et al. (2002) Blood 99:768; Coutinho, A. et al. (2001) Immunol. Rev. 182:89; Schwartz, R H (1990) Science 248:1349; Miller, J. F. et al. (1989) Immunology Today 10:53).

Self-tolerance is an acquired process that has to be learned by the lymphocytes of each individual. It occurs in part because lymphocytes pass through a stage in their development when an encounter with antigen presented by antigen-presenting cells (APCs) leads to their death or inactivation in a process known as positive and negative selection (see, e.g., Debatin K M (2001) Ann. Hematol. 80 Suppl 3:B29; Abbas, A. (1991), supra). Thus, potentially self-recognizing lymphocytes come into contact with self-antigens at this stage of functional immaturity and are prevented from developing to a stage at which they would be able to respond to self-antigens. Autoimmunity arises when abnormalities in the induction or maintenance of self-tolerance occur that result in a loss of tolerance to a particular antigen(s) and a subsequent attack by the host's immune system on the host's tissues that express the antigen(s) (see, e.g., Boyton R J et al. (2002) Clin. Exp. Immunol. 127:4; Hagiwara E. (2001) Ryumachi 41:888; Burt R K et al. (2992) Blood 99:768).

The ability of the immune system to distinguish between self and foreign antigens also plays a critical role in tissue transplantation. The success of a transplant depends on preventing the immune system of the host recipient from recognizing the transplant as foreign and, in some cases, preventing the graft from recognizing the host tissues as foreign. For example, when a host receives a bone marrow transplant, the transplanted bone marrow may recognize the new host as foreign, resulting in graft versus host disease (GVHD). Consequently, the survival of the host depends on preventing both the rejection of the donor marrow as well as rejection of the host by the graft immune reaction (see, e.g., Waldmann H et al. (2001) Int. Arch. Allergy Immunol. 126:11).

Currently, deleterious immune reactions that result in autoimmune diseases and transplant rejections are prevented or treated using agents such as steroids, azathioprine, anti-T cell antibodies, and more recently, monoclonal antibodies to T cell subpopulations. Immunosuppressive drugs such as cyclosporin A (CsA), rapamycin, desoxyspergualine and FK-506 are also widely used.

Nonspecific immune suppression agents, such as steroids and antibodies to lymphocytes, put the host at increased risk for opportunisitic infection and development of tumors. Moreover, many immunosuppressive drugs result in bone demineralization within the host (see, e.g., Chhajed P N et al. (2002) Indian J. Chest Dis. Allied 44:31; Wijdicks E F (2001) Liver Transpl. 7:937; Karamehic J et al. (2001) Med. Arh. 55:243; U.S. Pat. No. 5,597,563 issued to Beschorner, W E and U.S. Pat. No. 6,071,897 issued to DeLuca H F et al.). Because of the major drawbacks associated with existing immunosuppressive modalities, there is a need for a new approach for treating immune disorders, e.g., for inducing immune tolerance in a host.

Thus, in another aspect, the invention provides a method for modulating the activity of an immune cell by contacting the cell with a vitamin D₃ compound of formula I or otherwise described herein.

In one embodiment the invention provides a method of modulating the expression of an immunoglobulin-like transcript 3 (ILT3) surface molecule in a cell, comprising contacting said cell with a vitamin D₃ compound of described herein above in an amount effective to modulate the expression of an immunoglobulin-like transcript 3 (ILT3) surface molecule in said cell. In certain embodiments, the cell is within a subject.

A related embodiment of the invention provides a method of inducing immunological tolerance in a subject, comprising administering to said subject a vitamin D₃ compound described herein above in an amount effective to modulate the expression of an ILT3 surface molecule, thereby inducing immunological tolerance in said subject.

Another embodiment of the invention provides a method for modulating immunosuppressive activity by an antigen-presenting cell, comprising contacting an antigen-presenting cell with a vitamin D₃ compound described herein above in an amount effective to modulate ILT3 surface molecule expression, thereby modulating said immunosuppressive activity by said antigen-presenting cell.

In certain embodiments, the target of the methods is an antigen-presenting cell. Antigen-presenting cells include dendritic cells, monocytes, and macrophages. In yet other embodiments, the expression of said immunoglobulin-like transcript 3 (ILT3) surface molecule is upregulated.

In one embodiment, the present invention provides a method for suppressing immune activity in an immune cell by contacting a pathological or non-pathological immune cell with an effective amount of a vitamin D₃ compound of the invention to thereby inhibit an immune response relative to the cell in the absence of the treatment. The present method can be performed on cells in culture, e.g., in vitro or ex vivo, or can be performed on cells present in an animal subject, e.g., as part of an in vivo therapeutic protocol. In vivo treatment can be carried out on a human or other animal subject.

The vitamin D₃ compounds of the invention can be tested initially in vitro for their inhibitory effects on T cell proliferation and secretory activity, as described in Reichel, H. et al., (1987) Proc. Natl. Acad. Sci. USA 84:3385-3389; Lemire, J. M. et al. (1985) J. Immunol 34:2032-2035. Alternatively, the immunosuppressive effects can be tested in vivo using the various animal models known in the art and summarized by Bouillon, R. et al. (1995) Endocrine Reviews 16(2) 232 (Tables 6 and 7). For examples, animal models for autoimmune disorders, e.g., lupus, thyroiditis, encephalitis, diabetes and nephritis are described in (Lemire J. M. (1992) J. Cell Biochem. 49:26-31; Koizumi T. et al. (1985) Int. Arch. Allergy Appl. Immunol. 77:396-404; Abe J. et al. (1990) Calcium Regulation and Bone Metabolism 146-151; Fournier C. et al. (1990) Clin. Immunol Immunopathol. 54:53-63; Lemire J. M. and Archer D.C. (1991) J. Clin. Invest. 87:1103-1107); Lemire, J. M. et al., (1994) Endocrinology 135 (6):2818-2821; Inaba M. et al. (1992) Metabolism 41:631-635; Mathieu C. et al. (1992) Diabetes 41:1491-1495; Mathieu C. et al. (1994) Diabetologia 37:552-558; Lillevang S. T. et al. (1992) Clin. Exp. Immunol. 88:301-306, among others). Models for characterizing immunosuppressuve activity during organ transplantation, e.g., skin graft, cardiac graft, islet graft, are described in Jordan S. C. et al. (1988) v Herrath D (eds) Molecular, Cellular and Clinical Endocrinology 346-347; Veyron P. et al. (1993) Transplant Immunol. 1:72-76; Jordan S.C. (1988) v Herrath D (eds) Molecular, Cellular and Clinical Endocrinology 334-335; Lemire J. M. et al. (1992) Transplantation 54:762-763; Mathieu C. et al. (1994) Transplant Proc. 26:3128-3129).

After identifying certain test compounds as effective suppressors of an immune response in vitro, these compounds can be used in vivo as part of a therapeutic protocol. Accordingly, another embodiment provides a method of suppressing an immune response, comprising administering to a subject a pharmaceutical preparation of a vitamin D₃ compounds of the invention, so as to inhibit immune reactions such as graft rejection, autoimmune disorders and inflammation.

For example, the subject vitamin D₃ compound of the invention can be used to inhibit responses in clinical situations where it is desirable to downmodulate T cell responses. For example, in graft-versus-host disease, cases of transplantation, autoimmune diseases (including, for example, diabetes mellitus, arthritis (including rheumatoid arthritis, juvenile rheumatoid arthritis, osteoarthritis, psoriatic arthritis), multiple sclerosis, encephalomyelitis, diabetes, myasthenia gravis, systemic lupus erythematosis, autoimmune thyroiditis, dermatitis (including atopic dermatitis and eczematous dermatitis), psoriasis, Sjogren's Syndrome, including keratoconjunctivitis sicca secondary to Sjögren's Syndrome, alopecia greata, allergic responses due to arthropod bite reactions, Crohn's disease, aphthous ulcer, iritis, conjunctivitis, keratoconjunctivitis, ulcerative colitis, asthma, allergic asthma, cutaneous lupus erythematosus, scleroderma, vaginitis, proctitis, drug eruptions, leprosy reversal reactions, erythema nodosum leprosum, autoimmune uveitis, allergic encephalomyelitis, acute necrotizing hemorrhagic encephalopathy, idiopathic bilateral progressive sensorineural hearing loss, aplastic anemia, pure red cell anemia, idiopathic thrombocytopenia, polychondritis, Wegener's granulomatosis, chronic active hepatitis, Stevens-Johnson syndrome, idiopathic sprue, lichen planus, Crohn's disease, Graves opthalmopathy, sarcoidosis, primary biliary cirrhosis, uveitis posterior, and interstitial lung fibrosis). Downmodulation of immune activity will also be desirable in cases of allergy such as, atopic allergy.

In other embodiments, the present invention provides methods and compositions for treating immune disorders, such as, for example, autoimmune disorders and transplant rejections, such as graft versus host disease (GVHD). These embodiments of the invention are based on the discovery that vitamin D compounds of the invention are able to modulate the expression of immunoglobulin-like transcript 3 (ILT3) on cells, e.g., antigen-presenting cells.

As described before, determination of a therapeutically effective immunosuppressive amount can be readily made by the attending clinician, as one skilled in the art, by the use of known techniques and by observing results obtained under analogous circumstances. Compounds which are determined to be effective in animals, e.g., dogs, rodents, may be extrapolated accordingly to humans by those skilled in the art. Starting dose/regimen used in animals can be estimated based on prior studies. For example, doses of vitamin D₃ compounds of the invention to treat autoimmune disorders in rodents can be initially estimated in the range of 0.1 g/kg/day to 1 g/kg/day, administered orally or by injection.

Those skilled in the art will know based upon the data obtained in animal studies, the dosage and route of administration in humans is expected to be similar to that in animals. Exemplary dose ranges to be used in humans are from 0.25 to 10 μg/day, preferably 0.5 to 5 μg/day per adult (U.S. Pat. No. 4,341,774).

D. Calcium and Phosphate Homeostasis

The present invention also relates to a method of treating in a subject a disorder characterized by deregulation of calcium metabolism. This method comprises contacting a pathological or non-pathological vitamin D₃ responsive cell with an effective amount of a vitamin D₃ compound of the invention to thereby directly or indirectly modulate calcium and phosphate homeostasis. Techniques for detecting calcium fluctuation in vivo or in vitro are known in the art.

Exemplary Ca⁺⁺ homeostasis related assays include assays that focus on the intestine where intestinal ⁴⁵Ca²⁺ absorption is determined either 1) in vivo (Hibberd K. A. and Norman A. W. (1969) Biochem. Pharmacol. 18:2347-2355; Hurwitz S. et al. (1967) J. Nutr. 91:319-323; Bickle D. D. et al. (1984) Endocrinology 114:260-267), or 2) in vitro with everted duodenal sacs (Schachter D. et al. (1961) Am. J. Physiol 200:1263-1271), or 3) on the genomic induction of calbindin-D₂₈ₖ in the chick or of calbindin-D₉ₖ in the rat (Thomasset M. et al. (1981) FEBS Lett. 127:13-16; Brehier A. and Thomasset M. (1990) Endocrinology 127:580-587). The bone-oriented assays include: 1) assessment of bone resorption as determined via the release of Ca²⁺ from bone in vivo (in animals fed a zero Ca²⁺ diet) (Hibberd K. A. and Norman A. W. (1969) Biochem. Pharmacol. 18:2347-2355; Hurwitz S. et al. (1967) J. Nutr. 91:319-323), or from bone explants in vitro (Bouillon R. et al. (1992) J. Biol. Chem. 267:3044-3051), 2) measurement of serum osteocalcin levels [osteocalcin is an osteoblast-specific protein that after its synthesis is largely incorporated into the bone matrix, but partially released into the circulation (or tissue culture medium) and thus represents a good market of bone formation or turnover] (Bouillon R. et al. (1992) Clin. Chem. 38:2055-2060), or 3) bone ash content (Norman A. W. and Wong R. G. (1972) J. Nutr. 102:1709-1718). Only one kidney-oriented assay has been employed. In this assay, urinary Ca²⁺ excretion is determined (Hartenbower D. L. et al. (1977) Walter de Gruyter, Berlin pp 587-589); this assay is dependent upon elevations in the serum Ca²⁺ level and may reflect bone Ca²⁺ mobilizing activity more than renal effects. Finally, there is a “soft tissue calcification” assay that can be used to detect the consequences of administration of a compound of the invention. In this assay a rat is administered an intraperitoneal dose of ⁴⁵Ca²⁺, followed by seven daily relative high doses of a compound of the invention; in the event of onset of a severe hypercalcemia, soft tissue calcification can be assessed by determination of the 45Ca²⁺ level. In all these assays, vitamin D₃ compounds of the invention are administered to vitamin D-sufficient or -deficient animals, as a single dose or chronically (depending upon the assay protocol), at an appropriate time interval before the end point of the assay is quantified.

In certain embodiments, vitamin D₃ compounds of the invention can be used to modulate bone metabolism. The language “bone metabolism” is intended to include direct or indirect effects in the formation or degeneration of bone structures, e.g., bone formation, bone resorption, etc., which may ultimately affect the concentrations in serum of calcium and phosphate. This term is also intended to include effects of vitamin D₃ compounds in bone cells, e.g. osteoclasts and osteoblasts, that may in turn result in bone formation and degeneration. For example, it is known in the art, that vitamin D₃ compounds exert effects on the bone forming cells, the osteoblasts through genomic and non-genomic pathways (Walters M. R. et al. (1982) J. Biol. Chem. 257:7481-7484; Jurutka P. W. et al. (1993) Biochemistry 32:8184-8192; Mellon W. S, and DeLuca H.F. (1980) J. Biol. Chem. 255:4081-4086). Similarly, vitamin D₃ compounds are known in the art to support different activities of bone resorbing osteoclasts such as the stimulation of differentiation of monocytes and mononuclear phagocytes into osteoclasts (Abe E. et al. (1988) J. Bone Miner Res. 3:635-645; Takahashi N. et al. (1988) Endocrinology 123:1504-1510; Udagawa N. et al. (1990) Proc. Natl. Acad. Sci. USA 87:7260-7264). Accordingly, vitamin D₃ compounds of the invention that modulate the production of bone cells can influence bone formation and degeneration.

The present invention provides a method for modulating bone cell metabolism by contacting a pathological or a non-pathological bone cell with an effective amount of a vitamin D₃ compound of the invention to thereby modulate bone formation and degeneration. The present method can be performed on cells in culture, e.g., in vitro or ex vivo, or can be performed in cells present in an animal subject, e.g., cells in vivo. Exemplary culture systems that can be used include osteoblast cell lines, e.g., ROS 17/2.8 cell. line, monocytes, bone marrow culture system (Suda T. et al. (1990) Med. Res. Rev. 7:333-366; Suda T. et al. (1992) J. Cell Biochem. 49:53-58) among others. Selected compounds can be further tested in vivo, for example, animal models of osteopetrosis and in human disease (Shapira F. (1993) Clin. Orthop. 294:34-44).

In a preferred embodiment, a method for treating osteoporosis is provided, comprising administering to a subject a pharmaceutical preparation of a vitamin D₃ compound of the invention to thereby ameliorate the condition relative to an untreated subject.

Vitamin D₃ compounds of the invention can be tested in ovarectomized animals, e.g., dogs, rodents, to assess the changes in bone mass and bone formation rates in both normal and estrogen-deficient animals. Clinical trials can be conducted in humans by attending clinicians to determine therapeutically effective amounts of the vitamin D₃ compounds of the invention in preventing and treating osteoporosis.

In other embodiments, therapeutic applications of the vitamin D₃ compounds of the invention include treatment of other diseases characterized by metabolic calcium and phosphate deficiencies. Exemplary of such diseases are the following: osteoporosis, osteodystrophy, osteomalacia, rickets, osteitis fibrosa cystica, renal osteodystrophy, osteosclerosis, anti-convulsant treatment, osteopenia, fibrogenesis-imperfecta ossium, secondary hyperparathyrodism, hypoparathyroidism, hyperparathyroidism, cirrhosis, obstructive jaundice, drug induced metabolism, medullary carcinoma, chronic renal disease, hypophosphatemic VDRR, vitamin D-dependent rickets, sarcoidosis, glucocorticoid antagonism, malabsorption syndrome, steatorrhea, tropical sprue, idiopathic hypercalcemia and milk fever.

E. Hormone Secretion

In yet another aspect, the present invention provides a method for modulating hormone secretion of a vitamin D₃-responsive cell, e.g., an endocrine cell. Hormone secretion includes both genomic and non-genomic activities of vitamin D₃ compounds of the invention that control the transcription and processing responsible for secretion of a given hormone e.g., parathyroid hormone (PTH), calcitonin, insulin, prolactin (PRL) and T in a vitamin D₃ responsive cell (Bouillon, R. et al. (1995) Endocrine Reviews 16(2):235-237).

The present method can be performed on cells in culture, e.g. in vitro or ex vivo, or on cells present in an animal subject, e.g., in vivo. Vitamin D₃ compounds of the invention can be initially tested in vitro using primary cultures of parathyroid cells. Other systems that can be used include the testing by prolactin secretion in rat pituitary tumor cells, e.g., GH4C1 cell line (Wark J. D. and Tashjian Jr. A. H. (1982) Endocrinology 111:1755-1757; Wark J. D. and Tashjian Jr. A. H. (1983) J. Biol. Chem. 258:2118-2121; Wark J. D. and Gurtler V. (1986) Biochem. J. 233:513-518) and TRH secretion in GH4C1 cells. Alternatively, the effects of vitamin D₃ compounds of the invention can be characterized in vivo using animals models as described in Nko M. et al. (1982) Miner Electrolyte Metab. 5:67-75; Oberg F. et al. (1993) J. Immunol. 150:3487-3495; Bar-Shavit Z. et al. (1986) Endocrinology 118:679-686; Testa U. et al. (1993) J. Immunol 150:2418-2430; Nakamaki T. et al. (1992) Anticancer Res. 12:1331-1337; Weinberg J. B. and Larrick J. W. (1987) Blood 70:994-1002; Chambaut-Guérin A. M. and Thomopoulos P. (1991) Eur. Cytokine New. 2:355; Yoshida M. et al. (1992) Anticancer Res. 12:1947-1952; Momparler R. L. et al. (1993) Leukemia 7:17-20; Eisman J. A. (1994) Kanis J A (eds) Bone and Mineral Research 2:45-76; Veyron P. et al. (1993) Transplant Immunol. 1:72-76; Gross M. et al. (1986) J Bone Miner Res. 1:457-467; Costa E. M. et al. (1985) Endocrinology 117:2203-2210; Koga M. et al. (1988) Cancer Res. 48:2734-2739; Franceschi R. T. et al. (1994) J. Cell Physiol. 123:401-409; Cross H. S. et al. (1993) Naunyn Schmiedebergs Arch. Pharmacol. 347:105-110; Zhao X. and Feldman D. (1993) Endocrinology 132:1808-1814; Skowronski R. J. et al. (1993) Endocrinology 132:1952-1960; Henry H. L. and Norman A. W. (1975) Biochem. Biophys. Res. Commun. 62:781-788; Wecksler W. R. et al. (1980) Arch. Biochem. Biophys. 201:95-103; Brumbaugh P. F. et al. (1975) Am. J. Physiol. 238:384-388; Oldham S. B. et al. (1979) Endocrinology 104; 248-254; Chertow B. S. et al. (1975) J. Clin Invest. 56:668-678; Canterbury J. M. et al. (1978) J. Clin. Invest. 61:1375-1383; Quesad J. M. et al. (1992) J. Clin. Endocrinol. Metab. 75:494-501.

In certain embodiments, the vitamin D₃ compounds of the present invention can be used to inhibit parathyroid hormone (PTH) processing, e.g., transcriptional, translational processing, and/or secretion of a parathyroid cell as part of a therapeutic protocol. Therapeutic methods using these compounds can be readily applied to all diseases, involving direct or indirect effects of PTH activity, e.g., primary or secondary responses.

Accordingly, therapeutic applications for the vitamin D₃ compounds of the invention include treating diseases such as secondary hyperparathyroidism of chronic renal failure (Slatopolsky E. et al. (1990) Kidney Int. 38:S41-S47; Brown A. J. et al. (1989) J. Clin. Invest. 84:728-732). Determination of therapeutically affective amounts and dose regimen can be performed by the skilled artisan using the data described in the art.

F. Protection Against Neuronal Loss

In yet another aspect, the present invention provides a method of protecting against neuronal loss by contacting a vitamin D₃ responsive cell, e.g., a neuronal cell, with a vitamin D₃ compound of the invention to prevent or retard neuron loss. The language “protecting against” is intended to include prevention, retardation, and/or termination of deterioration, impairment, or death of a neurons.

Neuron loss can be the result of any condition of a neuron in which its normal function is compromised. Neuron deterioration can be the result of any condition which compromises neuron function which is likely to lead to neuron loss. Neuron function can be compromised by, for example, altered biochemistry, physiology, or anatomy of a neuron. Deterioration of a neuron may include membrane, dendritic, or synaptic changes which are detrimental to normal neuronal functioning. The cause of the neuron deterioration, impairment, and/or death may be unknown. Alternatively, it may be the result of age- and/or disease-related changes which occur in the nervous system of a subject.

When neuron loss is described herein as “age-related”, it is intended to include neuron loss resulting from known and unknown bodily changes of a subject which are associated with aging. When neuron loss is described herein as “disease-related”, it is intended to include neuron loss resulting from known and unknown bodily changes of a subject which are associated with disease. It should be understood, however, that these terms are not mutually exclusive and that, in fact, many conditions that result in the loss of neurons are both age- and disease-related.

Exemplary age-related diseases associated with neuron loss and changes in neuronal morphology include, for example, Alzheimer's Disease, Pick's Disease, Parkinson's Disease, Vascular Disease, Huntington's Disease, and Age-Associated Memory Impairment. In Alzheimer's Disease patients, neuron loss is most notable in the hippocampus, frontal, parietal, and anterior temporal cortices, amygdala, and the olfactory system. The most prominently affected zones of the hippocampus include the CA1 region, the subiculum, and the entorhinal cortex. Memory loss is considered the earliest and most representative cognitive change because the hippocampus is well known to play a crucial role in memory. Pick's Disease is characterized by severe neuronal degeneration in the neocortex of the frontal and anterior temporal lobes which is sometimes accompanied by death of neurons in the striatum. Parkinson's Disease can be identified by the loss of neurons in the substantia nigra and the locus ceruleus. Huntington's Disease is characterized by degeneration of the intrastriatal and cortical cholinergic neurons and GABA-ergic neurons. Parkinson's and Huntington's Diseases are usually associated with movement disorders, but often show cognitive impairment (memory loss) as well.

Age-Associated Memory Impairment (AAMI) is another age-associated disorder that is characterized by memory loss in healthy, elderly individuals in the later decades of life. Crook, T. et al. (1986) Devel. Neuropsych. 2(4):261-276. Presently, the neural basis for AAMI has not been precisely defined. However, neuron death with aging has been reported to occur in many species in brain regions implicated in memory, including cortex, hippocampus, amygdala, basal ganglia, cholinergic basal forebrain, locus ceruleus, raphe nuclei, and cerebellum. Crook, T. et al. (1986) Devel. Neuropsych. 2(4):261-276.

Vitamin D₃ compounds of the invention can protect against neuron. loss by genomic or non-genomic mechanisms. Nuclear vitamin D₃ receptors are well known to exist in the periphery but have also been found in the brain, particularly in the hippocampus and neocortex. Non-genomic mechanisms may also prevent or retard neuron loss by regulating intraneuronal and/or peripheral calcium and phosphate levels. Furthermore, vitamin D₃ compounds of the invention may protect against neuronal loss by acting indirectly, e.g., by modulating serum PTH levels. For example, a positive correlation has been demonstrated between serum PTH levels and cognitive decline in Alzheimer's Disease.

The present method can be performed on cells in culture, e.g. in vitro or ex vivo, or on cells present in an animal subject, e.g., in vivo. Vitamin D₃ compounds of the invention can be initially tested in vitro using neurons from embryonic rodent pups (See e.g. U.S. Pat. No. 5,179,109-fetal rat tissue culture), or other mammalian (See e.g. U.S. Pat. No. 5,089,517-fetal mouse tissue culture) or non-mammalian animal models. These culture systems have been used to characterize the protection of peripheral, as well as, central nervous system neurons in animal or tissue culture models of ischemia, stroke, trauma, nerve crush, Alzheimer's Disease, Pick's Disease, and Parkinson's Disease, among others. Examples of in vitro systems to study the prevention of destruction of neocortical neurons include using in vitro cultures of fetal mouse neurons and glial cells previously exposed to various glutamate agonists, such as kainate, NMDA, and α-amino-3-hydroxy-5-methyl-4-isoxazolepronate (AMPA). U.S. Pat. No. 5,089,517. See also U.S. Pat. No. 5,170,109 (treatment of rat cortical/hippocampal neuron cultures with glutamate prior to treatment with neuroprotective compound); U.S. Pat. Nos. 5,163,196 and 5,196,421 (neuroprotective excitatory amino acid receptor antagonists inhibit glycine, kainate, AMPA receptor binding in rats).

Alternatively, the effects of vitamin D₃ compounds of the invention can be characterized in vivo using animals models. Neuron deterioration in these model systems is often induced by experimental trauma or intervention (e.g. application of toxins, nerve crush, interruption of oxygen supply).

G. Smooth Muscle Cells

In yet another aspect, the present invention provides a method of modulating the activity of a vascular smooth muscle cell by contacting a vitamin D₃-responsive smooth muscle cell with a vitamin D₃ compound of the invention to activate or, preferably, inhibit the activity of the cell. The language “activity of a smooth muscle cell” is intended to include any activity of a smooth muscle cell, such as proliferation, migration, adhesion and/or metabolism.

In certain embodiments, the vitamin D₃ compounds of the invention can be used to treat diseases and conditions associated with aberrant activity of a vitamin D₃-responsive smooth muscle cell. For example, the present invention can be used in the treatment of hyperproliferative vascular diseases, such as hypertension induced vascular remodeling, vascular restenosis and atherosclerosis. In other embodiments, the compounds of the present invention can be used in treating disorders characterized by aberrant metabolism of a vitamin D₃-responsive smooth muscle cell, e.g., arterial hypertension.

The present method can be performed on cells in culture, e.g. in vitro or ex vivo, or on cells present in an animal subject, e.g., in vivo. Vitamin D₃ compounds of the invention can be initially tested in vitro as described in Catellot et al. (1982), J. Biol. Chem. 257(19): 11256.

H. Suppression of Renin Expression and Treatment of Hypertension

The compounds of the present invention control blood pressure by the suppression of rennin expression and are useful as antihypertensive agents. Renin-angiotensin regulatory cascade plays a significant role in the regulation of blood pressure, electrolyte and volume homeostasis (Y. C. Li, Abstract, DeLuca Symposium on Vitamin D₃, Tauc, N. Mex., Jun. 15-Jun. 19, 2002, p. 18). Thus, the invention provides a method of treating a subject for hypertension. The method comprises administering to said subject an effective amount of a Gemini vitamin D₃ compound, such that said subject is treated for hypertension In accordance with an embodiment of the method, the Gemini vitamin D₃ compound suppresses expression of renin, thereby treating the subject for hypertension.

Gemini vitamin D₃ compounds useful in the treatment of hypertension are compounds having formula II:

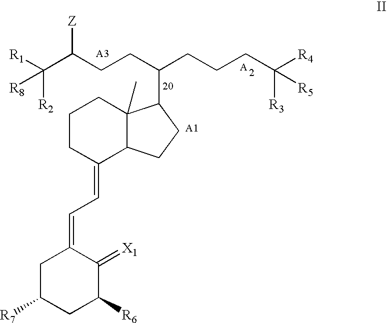

wherein:

A₁ is a single or a double bond;

A₂ is a single, a double or a triple bond;

A₃ is a single bond, an E-double bond, a Z-double bond or a triple bond, provided Z is absent when A₃ is a triple bond;

R₁, R₂, R₃ and R₄ are each independently C₁-C₄ alkyl, C₁-C₄ deuteroalkyl, hydroxyalkyl, or haloalkyl; or R₁ and R₂ together with C₂₅ form a C₁-C₄ cycloalkyl or cyclohaloalkyl; or R₃ and R₄ together with C₂₅ form a C₁-C₄ cycloalkyl or cyclohaloalkyl;

R₅, R₇ and R₈ are each independently hydroxyl, OC(O)C₁-C₄ alkyl, OC(O)hydroxyalkyl, or OC(O)haloalkyl;

R₆ is hydrogen, hydroxyl, halogen, OC(O)C₁-C₄ alkyl, OC(O)hydroxyallyl, or OC(O)haloalkyl;

X₁ is H₂ or CH₂;

Z is hydrogen, —OH, ═O, —SH, or —NH₂;

and pharmaceutically acceptable esters, salts, and prodrugs thereof.

In certain embodiments, the haloalkyl, the cyclohaloalkyl and the halogen recited in formula II are fluoroalkyl, cyclofluoroalky and fluorine, respectively.

In certain embodiments, the method further comprises obtaining the Gemini vitamin D₃ compound of formula II.

Specific compounds of formula II include the following Gemini vitamin D₃ compounds:

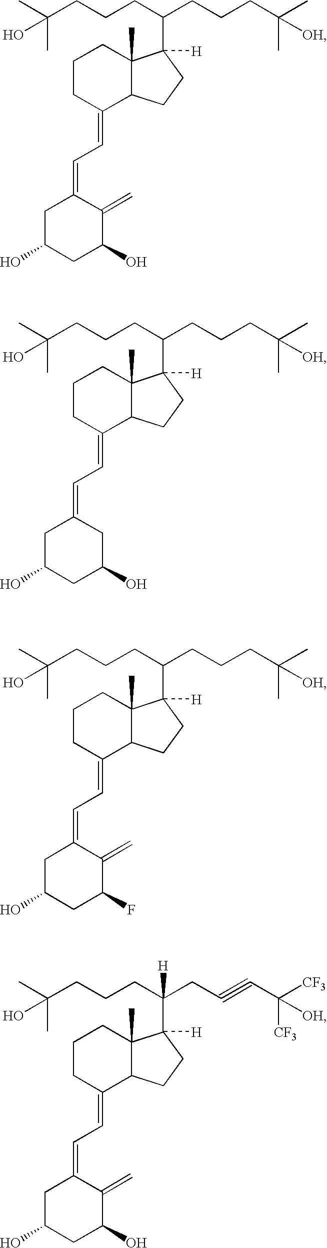

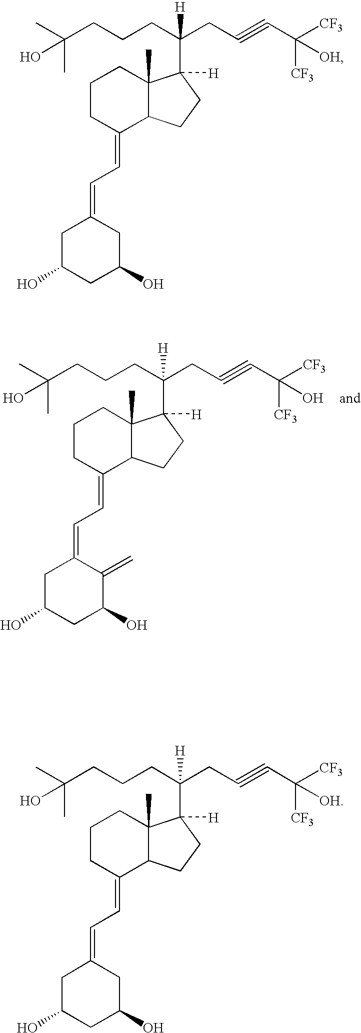

Other specific compounds include 1,25-Dihydroxy-21-(2R,3-dihydroxy-3-methyl-butyl)-20R-cholecalciferol, 1,25-Dihydroxy-21-(2R,3-dihydroxy-3-methyl-butyl)-20S-cholecalciferol, 1,25-Dihydroxy-21-(2R,3-dihydroxy-3-methyl-butyl)-20S-19-nor-cholecalciferol, 1,25-Dihydroxy-20S-21-(3-hydroxy-3-methyl-butyl)-24-keto-19-nor-cholecalciferol, 1,25-Dihydroxy-20S-21-(3-hydroxy-3-methyl-butyl)-24-keto-cholecalciferol, 1-Dihydroxy-21(3-hydroxy-3-trifluoromethyl-4-trifluoro-butynyl)-26,27-hexadeutero-19-nor-20S-cholecalciferol or 1,25-Dihydroxy-21(3-hydroxy-3-trifluoromethyl-4-trifluoro-butynyl)-26,27-hexadeutero-20S-cholecalciferol.

Particularly advantageous compounds for use in the method include 1,25-Dihydroxy-21-(2R,3-dihydroxy-3-methyl-butyl)-20R-cholecalciferol, or 1,25-Dihydroxy-21-(2R,3-dihydroxy-3-methyl-butyl)-20S-cholecalciferol.

In a related embodiment, the invention provides a method of suppressing renin expression in a subject comprising administering to a subject an effective amount of a Gemini vitamin D₃ compound such that renin expression in said subject is suppressed. The Gemini vitamin D₃ compounds include the compounds of formula II described above.

I. Treatment of Urogenital Disorders

The invention also provides a method for treating a subject for a urogenital disorder. The method comprises administering to the subject an effective amount of a vitamin D₃ compound of formula I above, such that the subject is treated for the urogential disorder.

In one embodiment, the urogenital disorder comprises bladder dysfunction, especially bladder dysfunction related to morphological bladder changes. The term bladder dysfunction as used in this embodiment does not include cancer of the bladder and associated urogenital organs.

Morphological bladder changes, including a progressive de-nervation and hypertrophy of the bladder wall are frequent histological findings in patients with different bladder disorders such as overactive bladder and clinical BPH. The increase in tension and/or strain on the bladder observed in these conditions has been shown to be associated with cellular and molecular alterations, e.g., in cytoskeletal and contractile proteins, in mitochondrial function, and in various enzyme activities of the smooth muscle cells. The growth of the bladder wall also involves alterations in its extracellular matrix and non-smooth muscle components.

These changes in the bladder are associated with the storage (irritative) symptoms, in particular frequency, urgency and nocturia. These symptoms affect the social, psychological, domestic, occupational, physical and sexual lives of the patients leading to a profound, negative impact on their quality of life.

Also included within urogenital disorders is benign prostatic hyperplasia (BPH). Thus the invention also provides a method for treatment of BPH comprising administering to a subject an effective amount of a vitamin D₃ compound of formula I above, such that the subject is treated for BPH.

BPH is commonly associated with enlargement of the gland (prostate) leading to bladder outlet obstruction (BOO) and symptoms secondary to BOO. However, BPH is also associated with morphological bladder changes, including a progressive denervation and hypertrophy of the bladder wall, the latter possibly as a consequence of increased functional demands. Thus, the compounds of the invention are useful for the treatment of storage (irritative) symptoms of BPH, as well as for bladder outlet obstruction caused by BPH.

Urorgenital disorders in accordance with the invention also include interstitial cystitis. Thus, in another embodiment, the invention also provides a method for treatment of interstitial cystitis comprising administering to a subject an effective amount of a vitamin D₃ compound of formula I above, such that the subject is treated for interstitial cystitis.

Interstitial cystitis (IC) is a chronic inflammatory bladder disease characterized by pelvic pain, urinary urgency and frequency. Unlike other bladder dysfunction conditions, IC is characterized by chronic inflammation of the bladder wall which is responsible for the symptomatology. In other words, the cause of the abnormal bladder contractility is the chronic inflammation and as a consequence the treatment should target this etiological component. In fact, the traditional treatment of bladder dysfunctions, like overactive bladder, with smooth muscle relaxant agents, is not effective in patients with IC.

### 4. Pharmaceutical Compositions

The invention also provides a pharmaceutical composition, comprising an effective amount a vitamin D₃ compound of formula I or otherwise described herein and a pharmaceutically acceptable carrier. In a further embodiment, the effective amount is effective to treat a vitamin D₃ associated state, as described previously.

In an embodiment, the vitamin D₃ compound is administered to the subject using a pharmaceutically-acceptable formulation, e.g., a pharmaceutically-acceptable formulation that provides sustained delivery of the vitamin D₃ compound to a subject for at least 12 hours, 24 hours, 36 hours, 48 hours, one week, two weeks, three weeks, or four weeks after the pharmaceutically-acceptable formulation is administered to the subject.

In certain embodiments, these pharmaceutical compositions are suitable for topical or oral administration to a subject. In other embodiments, as described in detail below, the pharmaceutical compositions of the present invention may be specially formulated for administration in solid or liquid form, including those adapted for the following: (1) oral administration, for example, drenches (aqueous or non-aqueous solutions or suspensions), tablets, boluses, powders, granules, pastes; (2) parenteral administration, for example, by subcutaneous, intramuscular or intravenous injection as, for example, a sterile solution or suspension; (3) topical application, for example, as a cream, ointment or spray applied to the skin; (4) intravaginally or intrarectally, for example, as a pessary, cream or foam; or (5) aerosol, for example, as an aqueous aerosol, liposomal preparation or solid particles containing the compound.

In certain embodiments, the subject is a mammal, e.g., a primate, e.g., a human.

The methods of the invention further include administering to a subject a therapeutically effective amount of a vitamin D₃ compound in combination with another pharmaceutically active compound. Examples of pharmaceutically active compounds include compounds known to treat autoimmune disorders, e.g., immunosuppressant agents such as cyclosporin A, rapamycin, desoxyspergualine, FK 506, steroids, azathioprine, anti-T cell antibodies and monoclonal antibodies to T cell subpopulations. Other pharmaceutically active compounds that may be used can be found in Harrison's Principles of Internal Medicine, Thirteenth Edition, Eds. T. R. Harrison et al. McGraw-Hill N.Y., NY; and the Physicians Desk Reference 50th Edition 1997, Oradell N.J., Medical Economics Co., the complete contents of which are expressly incorporated herein by reference. The angiogenesis inhibitor compound and the pharmaceutically active compound may be administered to the subject in the same pharmaceutical composition or in different pharmaceutical compositions (at the same time or at different times).

The phrase “pharmaceutically acceptable” is refers to those vitamin D₃ compounds of the present invention, compositions containing such compounds, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The phrase “pharmaceutically-acceptable carrier” includes pharmaceutically-acceptable material, composition or vehicle, such as a liquid or solid filler, diluent, excipient, solvent or encapsulating material, involved in carrying or transporting the subject chemical from one organ, or portion of the body, to another organ, or portion of the body. Each carrier must be “acceptable” in the sense of being compatible with the other ingredients of the formulation and not injurious to the patient. Some examples of materials which can serve as pharmaceutically-acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations.

Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

Examples of pharmaceutically-acceptable antioxidants include: (1) water soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHI), lecithin, propyl gallate, alpha-tocopherol, and the like; and (3) metal chelating agents, such as citric acid, ethylenediamine tetraacetic acid (EDTA), sorbitol, tartaric acid, phosphoric acid, and the like.

Compositions containing a vitamin D₃ compound(s) include those suitable for oral, nasal, topical (including buccal and sublingual), rectal, vaginal, aerosol and/or parenteral administration. The compositions may conveniently be presented in unit dosage form and may be prepared by any methods well known in the art of pharmacy. The amount of active ingredient which can be combined with a carrier material to produce a single dosage form will vary depending upon the host being treated, the particular mode of administration. The amount of active ingredient which can be combined with a carrier material to produce a single dosage form will generally be that amount of the compound which produces a therapeutic effect. Generally, out of one hundred percent, this amount will range from about 1 percent to about ninety-nine percent of active ingredient, preferably from about 5 percent to about 70 percent, most preferably from about 10 percent to about 30 percent.

Methods of preparing these compositions include the step of bringing into association a vitamin D₃ compound(s) with the carrier and, optionally, one or more accessory ingredients. In general, the formulations are prepared by uniformly and intimately bringing into association a vitamin D₃ compound with liquid carriers, or finely divided solid carriers, or both, and then, if necessary, shaping the product.

Compositions of the invention suitable for oral administration may be in the form of capsules, cachets, pills, tablets, lozenges (using a flavored basis, usually sucrose and acacia or tragacanth), powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouth washes and the like, each containing a predetermined amount of a vitamin D₃ compound(s) as an active ingredient. A compound may also be administered as a bolus, electuary or paste.

In solid dosage forms of the invention for oral administration (capsules, tablets, pills, dragees, powders, granules and the like), the active ingredient is mixed with one or more pharmaceutically-acceptable carriers, such as sodium citrate or dicalcium phosphate, and/or any of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds; (7) wetting agents, such as, for example, acetyl alcohol and glycerol monostearate; (8) absorbents, such as kaolin and bentonite clay; (9) lubricants, such a talc, calcium stearate, magnesium stearate, solid polyethylene glycols, sodium lauryl sulfate, and mixtures thereof; and (10) coloring agents. In the case of capsules, tablets and pills, the pharmaceutical compositions may also comprise buffering agents. Solid compositions of a similar type may also be employed as fillers in soft and hard-filled gelatin capsules using such excipients as lactose or milk sugars, as well as high molecular weight polyethylene glycols and the like.

A tablet may be made by compression or molding, optionally with one or more accessory ingredients. Compressed tablets may be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made by molding in a suitable machine a mixture of the powdered active ingredient moistened with an inert liquid diluent.

The tablets, and other solid dosage forms of the pharmaceutical compositions of the present invention, such as dragees, capsules, pills and granules, may optionally be scored or prepared with coatings and shells, such as enteric coatings and other coatings well known in the pharmaceutical-formulating art. They may also be formulated so as to provide slow or controlled release of the active ingredient therein using, for example, hydroxypropylmethyl cellulose in varying proportions to provide the desired release profile, other polymer matrices, liposomes and/or microspheres. They may be sterilized by, for example, filtration through a bacteria-retaining filter, or by incorporating sterilizing agents in the form of sterile solid compositions which can be dissolved in sterile water, or some other sterile injectable medium immediately before use. These compositions may also optionally contain opacifying agents and may be of a composition that they release the active ingredient(s) only, or preferentially, in a certain portion of the gastrointestinal tract, optionally, in a delayed manner. Examples of embedding compositions which can be used include polymeric substances and waxes. The active ingredient can also be in micro-encapsulated form, if appropriate, with one or more of the above-described excipients.

Liquid dosage forms for oral administration of the vitamin D₃ compound(s) include pharmaceutically-acceptable emulsions, microemulsions, solutions, suspensions, syrups and elixirs. In addition to the active ingredient, the liquid dosage forms may contain inert diluents commonly used in the art, such as, for example, water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof.

In addition to inert diluents, the oral compositions can include adjuvants such as wetting agents, emulsifying and suspending agents, sweetening, flavoring, coloring, perfuming and preservative agents.

Suspensions, in addition to the active vitamin D₃ compound(s) may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Pharmaceutical compositions of the invention for rectal or vaginal administration may be presented as a suppository, which may be prepared by mixing one or more vitamin D₃ compound(s) with one or more suitable nonirritating excipients or carriers comprising, for example, cocoa butter, polyethylene glycol, a suppository wax or a salicylate, and which is solid at room temperature, but liquid at body temperature and, therefore, will melt in the rectum or vaginal cavity and release the active agent.

Compositions of the present invention which are suitable for vaginal administration also include pessaries, tampons, creams, gels, pastes, foams or spray formulations containing such carriers as are known in the art to be appropriate.

Dosage forms for the topical or transdermal administration of a vitamin D₃ compound(s) include powders, sprays, ointments, pastes, creams, lotions, gels, solutions, patches and inhalants. The active vitamin D₃ compound(s) may be mixed under sterile conditions with a pharmaceutically-acceptable carrier, and with any preservatives, buffers, or propellants which may be required.

The ointments, pastes, creams and gels may contain, in addition to vitamin D₃ compound(s) of the present invention, excipients, such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays can contain, in addition to a vitamin D₃ compound(s), excipients such as lactose, talc, silicic acid, aluminum hydroxide, calcium silicates and polyamide powder, or mixtures of these substances. Sprays can additionally contain customary propellants, such as chlorofluorohydrocarbons and volatile unsubstituted hydrocarbons, such as butane and propane.

The vitamin D₃ compound(s) can be alternatively administered by aerosol. This is accomplished by preparing an aqueous aerosol, liposomal preparation or solid particles containing the compound. A nonaqueous (e.g., fluorocarbon propellant) suspension could be used. Sonic nebulizers are preferred because they minimize exposing the agent to shear, which can result in degradation of the compound.

Ordinarily, an aqueous aerosol is made by formulating an aqueous solution or suspension of the agent together with conventional pharmaceutically-acceptable carriers and stabilizers. The carriers and stabilizers vary with the requirements of the particular compound, but typically include nonionic surfactants (Tweens, Pluronics, or polyethylene glycol), innocuous proteins like serum albumin, sorbitan esters, oleic acid, lecithin, amino acids such as glycine, buffers, salts, sugars or sugar alcohols. Aerosols generally are prepared from isotonic solutions.

Transdermal patches have the added advantage of providing controlled delivery of a vitamin D₃ compound(s) to the body. Such dosage forms can be made by dissolving or dispersing the agent in the proper medium. Absorption enhancers can also be used to increase the flux of the active ingredient across the skin. The rate of such flux can be controlled by either providing a rate controlling membrane or dispersing the active ingredient in a polymer matrix or gel.

Ophthalmic formulations, eye ointments, powders, solutions and the like, are also contemplated as being within the scope of this invention.

Pharmaceutical compositions of this invention suitable for parenteral administration comprise one or more vitamin D₃ compound(s) in combination with one or more pharmaceutically-acceptable sterile isotonic aqueous or nonaqueous solutions, dispersions, suspensions or emulsions, or sterile powders which may be reconstituted into sterile injectable solutions or dispersions just prior to use, which may contain antioxidants, buffers, bacteriostats, solutes which render the formulation isotonic with the blood of the intended recipient or suspending or thickening agents.

Examples of suitable aqueous and nonaqueous carriers which may be employed in the pharmaceutical compositions of the invention include water, ethanol, polyols (such as glycerol, propylene glycol, polyethylene glycol, and the like), and suitable mixtures thereof vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions may also contain adjuvants such as preservatives, wetting agents, emulsifying agents and dispersing agents. Prevention of the action of microorganisms may be ensured by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents which delay absorption such as aluminum monostearate and gelatin.

In some cases, in order to prolong the effect of a drug, it is desirable to slow the absorption of the drug from subcutaneous or intramuscular injection. This may be accomplished by the use of a liquid suspension of crystalline or amorphous material having poor water solubility. The rate of absorption of the drug then depends upon its rate of dissolution which, in turn, may depend upon crystal size and crystalline form. Alternatively, delayed absorption of a parenterally-administered drug form is accomplished by dissolving or suspending the drug in an oil vehicle.

Injectable depot forms are made by forming microencapsule matrices of vitamin D₃ compound(s) in biodegradable polymers such as polylactide-polyglycolide. Depending on the ratio of drug to polymer, and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions which are compatible with body tissue.

When the vitamin D₃ compound(s) are administered as pharmaceuticals, to humans and animals, they can be given per se or as a pharmaceutical composition containing, for example, 0.1 to 99.5% (more preferably, 0.5 to 90%) of active ingredient in combination with a pharmaceutically-acceptable carrier.

Regardless of the route of administration selected, the vitamin D₃ compound(s), which may be used in a suitable hydrated form, and/or the pharmaceutical compositions of the present invention, are formulated into pharmaceutically-acceptable dosage forms by conventional methods known to those of skill in the art.

Actual dosage levels and time course of administration of the active ingredients in the pharmaceutical compositions of this invention may be varied so as to obtain an amount of the active ingredient which is effective to achieve the desired therapeutic response for a particular patient, composition, and mode of administration, without being toxic to the patient. An exemplary dose range is from 0.1 to 10 mg per day.

A preferred dose of the vitamin D₃ compound for the present invention is the maximum that a patient can tolerate and not develop serious hypercalcemia. Preferably, the vitamin D₃ compound of the present invention is administered at a concentration of about 0.001 μg to about 100 μg per kilogram of body weight, about 0.001-about 10 μg/kg or about 0.001 μg-about 100 μg/kg of body weight. Ranges intermediate to the above-recited values are also intended to be part of the invention.

### 5. Synthesis of Compounds of the Invention

Compounds of the invention can be synthesized by methods described in this section, the examples, and the chemical literature.

### A. Synthesis of 24-Hydroxyl Gemini Vitamin D₃ Compounds

Schemes 1-5 below graphically depict the reaction steps for the synthesis of the 24-hydroxyl Gemini vitamin D₃ compounds of the invention. For the synthesis of 24-hydroxyl geminal vitamin D₃ compounds 2, 3 and 38 the convergent and Wittig-Horner reaction using the Lythgoe phosphine oxide coupling protocol was used (Scheme 1). Two elaborated ketones 34 and 27 were each linked to the functionalized (2-cyclohexylethenyl)diphenylphosphine oxide 28. A single step removed all five silyl protecting groups in 35 and 29 and lead to the target compounds 2 and 3.

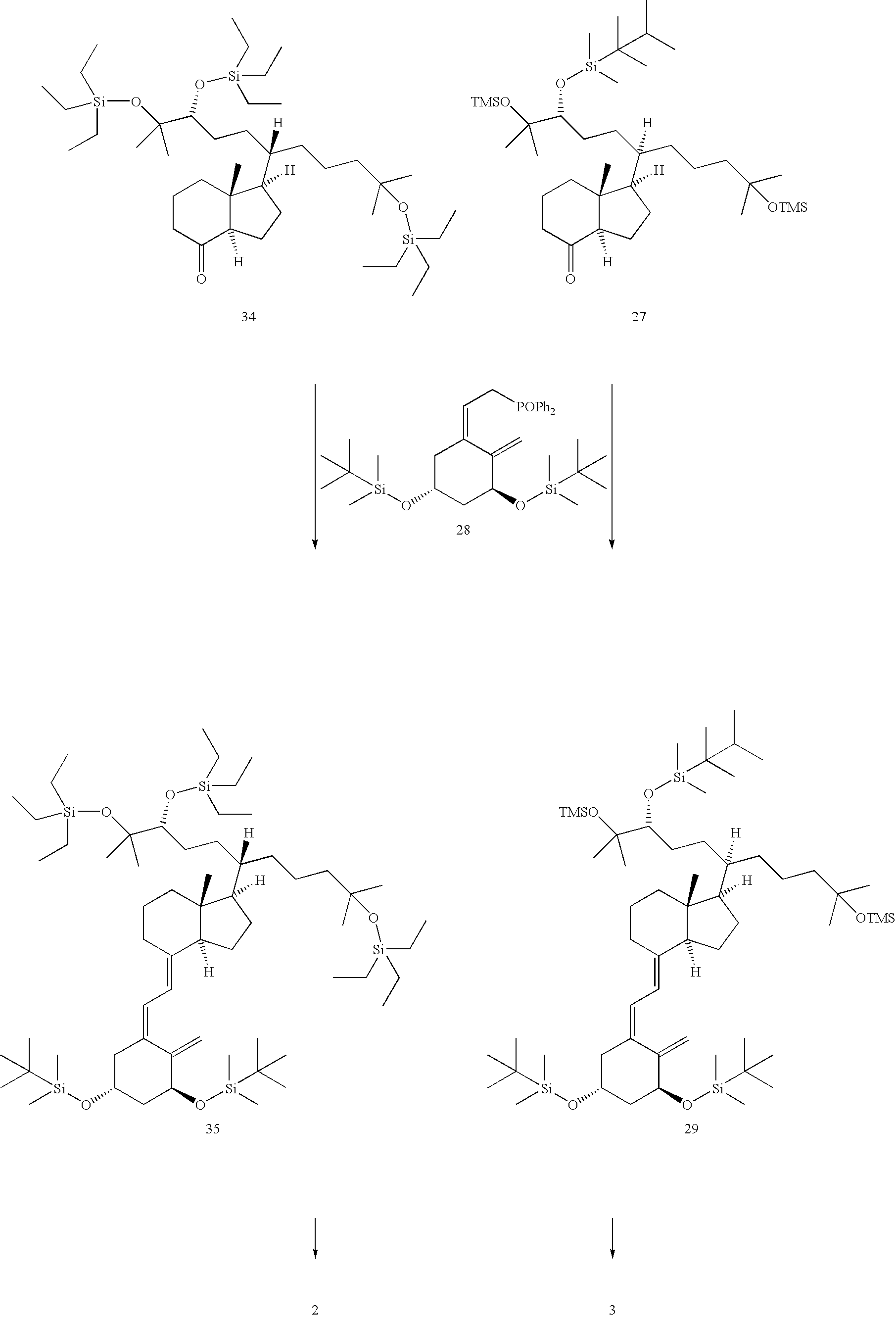

The synthesis of the required ketones commenced with the 4-O-(TBDMS) Lythgoe diol 4 whose conversion to the alkenol 5 has already been described (Maehr, H. et al. Symposium on Vitamin D, Taos, N. Mex., Jun. 15-19, 2002, Abstract p. 42.). A subsequent hydroboration gave the epimeric pair 6 and 7 that was separated by chromatography and obtained in a ratio of 3/2.

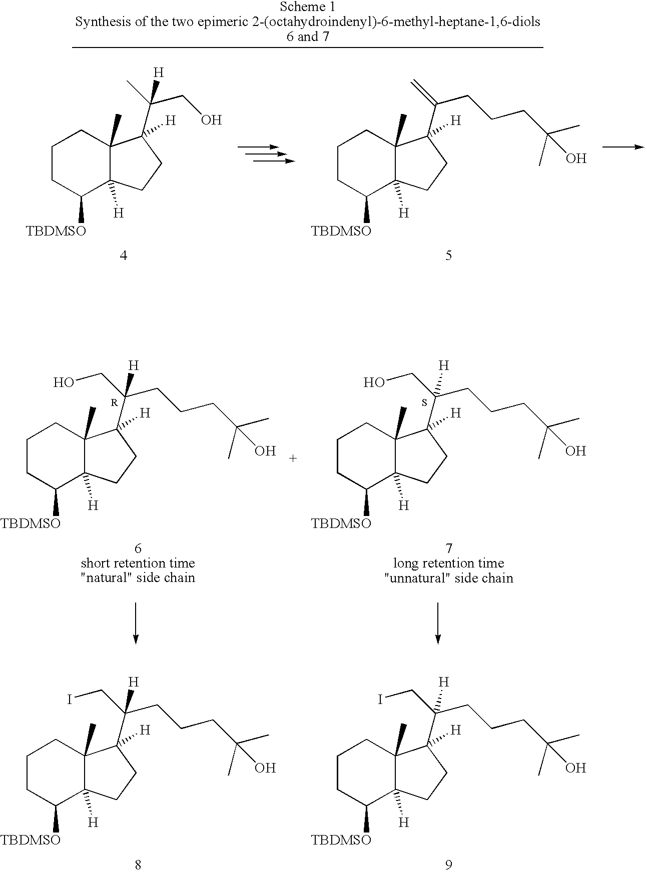

The diols 6 and 7 were then converted to the iodo alcohols 8 and 9 which served not only as key intermediates for the synthesis of 2 and 3, but also for the stereochemical elucidation of the two hydroboration products from which they were derived. In pursuit of the latter task, the iodo alcohol 8, derived from the diol with the shorter chromatographic retention time, was reacted with lithium acetylide to furnish an acetylene derivative that was identical with 14 and different from its 6(R) epimer. Both 14, representing the 6(S) configuration, and the corresponding 6(R)-epimer, were previously synthesized. In this cascade toward 14, alkenol 5 was subjected to an ene-reaction with formaldehyde, the resulting mixture of alkenediols 10a and 10b was hydrogenated to furnish the epimeric diol pair 11 and 12 that also could be separated by chromatography. The isomer 11, exhibiting the shorter chromatographic retention time than the epimer 12, was oxidized to the aldehyde 13 and further converted to the acetylene 14. Silylation of the tertiary hydroxyl group and a subsequent condensation with hexafluoroacetone gave 15a Maehr, H. et al. Symposium on Vitamin D, Taos, N. Mex., Jun. 15-19, 2002, Abstract p. 42). The two protective silyl groups were then removed to produce 15b. This triol was oxidized to the ketone 16 whose configuration was determined by crystal analysis and shown to have the S-configuration at the stereocenter equivalent to C-20 in the vitamin D series. In view of the availability of the 6(R)-epimer of 14 via 12, and the remarkably different ¹H NMR spectra of 14 and its 6(R)-epimer, the alkynol derived from 8 was identified as 14. The hydroboration product 6 was regarded as the R-isomer with respect to the stereocenter in the side-chain assembly.

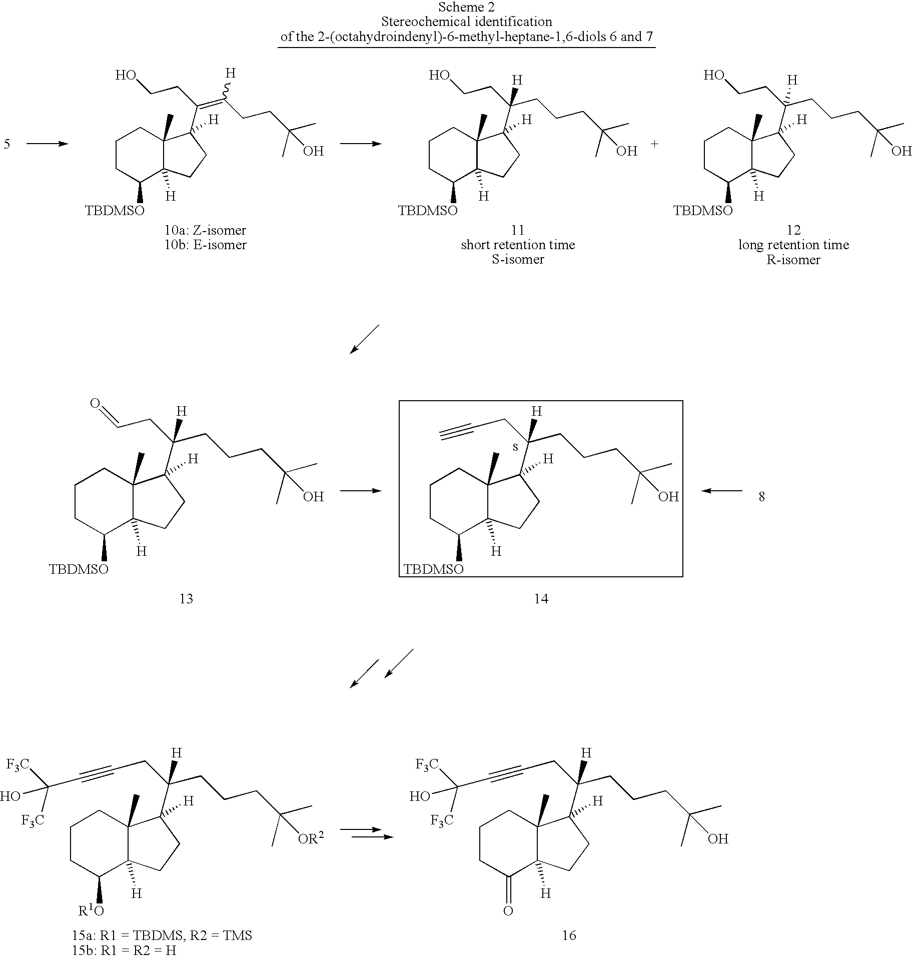

### Synthesis of 1,25-Dihydroxy-21-(2R,3-dihydroxy-3-methyl-butyl)-20R-cholecalciferol (3)

The syntheses of 2 and 3 were achieved by two different routes. The sequence of step leading to 3 commenced with the conversion of diol 7 to the iodo alcohol 9. This compound was treated sodium benzenesulfinate to give 19a, then silylated with 1-(trimethylsilyl)imidazole (Scheme 3). For the remaining synthetic steps toward 3, the intermittent protection of the vicinal diol by isopropylidination served for the regiospecific blocking of the 4-hydroxy group as an O-acetyl derivative as outlined in Scheme 3 (Hatakeyama, S. et al. Steroids 200, 66, 267-276). Reacting 19b with the 2-oxiranyl-2-propanol, prepared in situ from 21, led to the diol 20 as an epimeric mixture which was subjected to reductive de-sulfonylation to give 22a but also some 22b which is the partial de-silylated 22a.

Rather than completely de-silylating this mixture to obtain the tetraol 22c directly, a selective removal of the trimethylsilyl group was performed which gave 22b as a crystalline intermediate and hence the opportunity of additional purification and characterization. A subsequent treatment with fluorosilicic acid then produced the tetraol 22c. Treatment of 22c with acetone and 2,2-dimethoxypropane with pyridinium tosylate as catalyst gave a mixture of 23a and a more polar material, the acetal 23. A brief aqueous treatment of this mixture converted this more polar substance quantitatively to 23a. A subsequent reaction with acetic anhydride in pyridine led to the O-acetyl compound 23b and 80% aqueous acetic acid at 68° C. hydrolyzed the acetal moiety within 2.5 h to produce 24. Selective O-silylation with thexyldimethylsilyl chloride proceeded regioselectively to 25a and a following treatment with 1-(trimethylsilyl)imidazole gave 25b. The 4-acetoxy group was removed by treatment with lithium aluminum hydride and the resulting alcohol 26 was oxidized to the ketone 27 using pyridinium dichromate. The standard coupling protocol (Lythgoe, B. Chem. Soc. Rev. 1981, 10, 449-475; Zhu, G. D. et al. Chem. Rev. 1995, 95 1877-1952; Dai, H. et al. Synthesis 1994, 1383), employing [(2Z)-2-[(3S,5R)-3,5-bis(tert-butyldimethylsilanyloxy)-2-methylenecyclohexylidene]ethyl]diphenylphosphine oxide (28) (as Wittig-Horner component (Baggiolini, E. G. et al. J. Org. Chem. 1986, 51, 3098), gave 29 and a single treatment with tetrabutylammonium fluoride furnished the target compound 3.

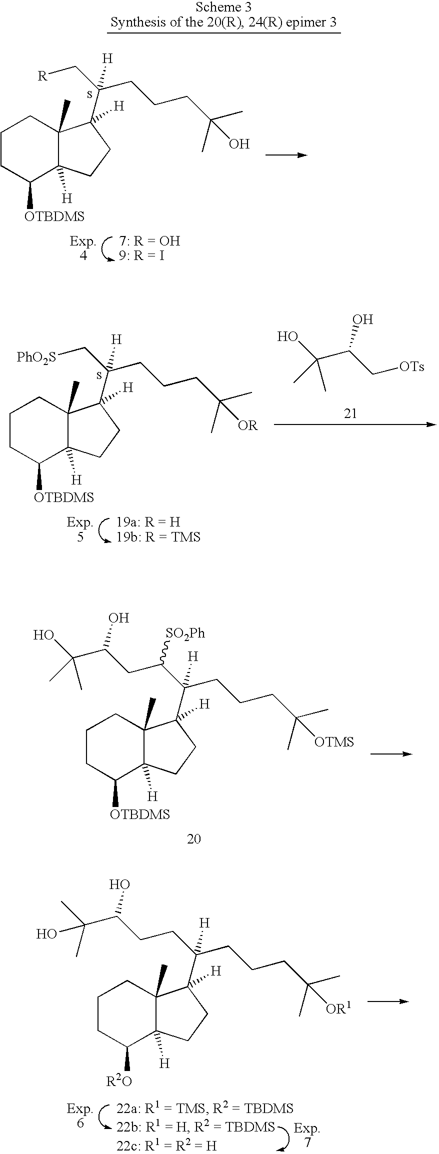

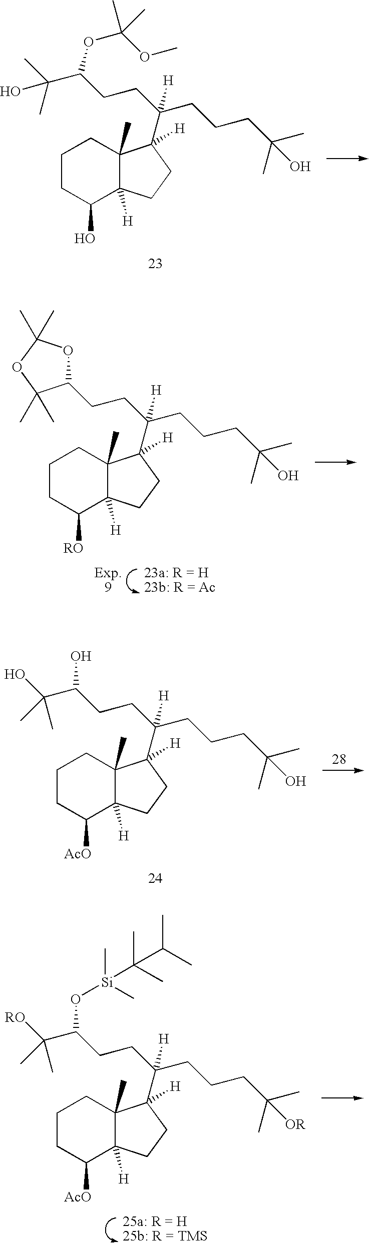

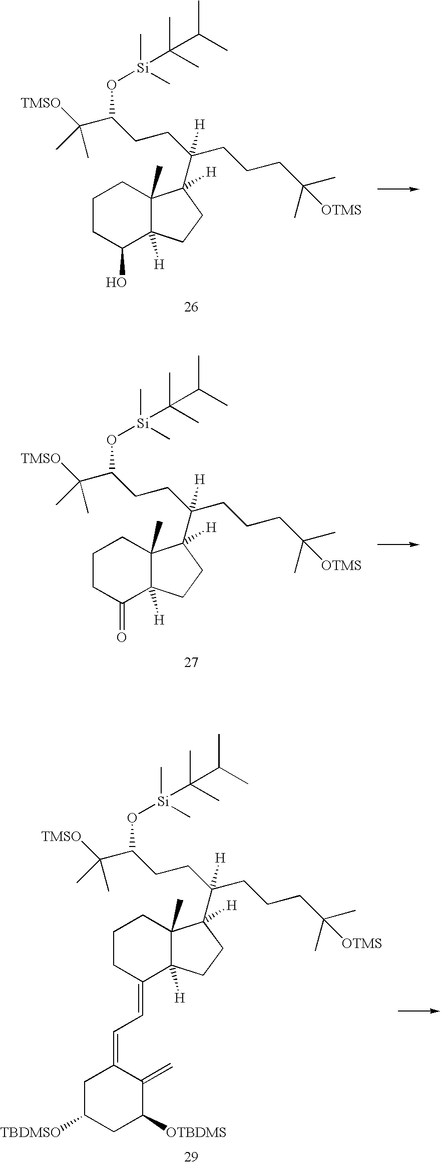

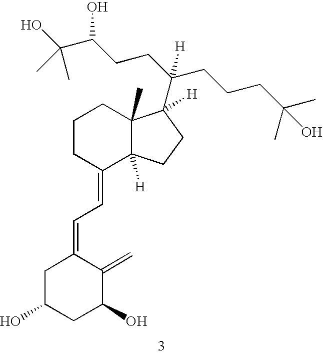

### Syntheses of 1,25-Dihydroxy-21(2R,3-dihydroxy-3-methyl-butyl)-20S-cholecalciferol (2)

Referring to Scheme 4, the conversion of diol 6 to 18a via the iodo alcohol 8, phenylsulfone 17a, trimethylsilyl derivative 17b and subsequent condensation of 17b with 21, was conducted in a fashion very similar to the corresponding steps described previously for the other epimer 22a. The trimethylsilyl group in 18a, however, was removed prior to reductive de-sulfonylation. Thus, the resulting 18b, upon treatment with sodium amalgam, led to the triol 30a directly and a subsequent reaction with fluorosilicic acid furnished the tetraol 30b. A considerable synthetic improvement was realized when this compound was treated with 4-methoxybenzylidene dimethylacetal and pyridinium tosylate to produce the oxolane 31 that was oxidized with pyridinium dichromate to ketone 32. It was shown that the 4-methoxybenzylidene acetal was sufficiently acid labile to permit its hydrolysis under conditions that do not compromise the trans ring-juncture of the 7a-methyl-octahydro-4-indenone system. Treatment with either 80% acetic acid or 1 N methanolic oxalic acid, the latter previously employed for the selective hydrolysis of the tertiary trimethylsilyl ether function in 22a, converted 32 the triol 33 that was treated with chlorotriethylsilane in N,N-dimethylformamide and imidazole to produce rapidly the disilyl intermediate. Further reaction of the second tertiary alcohol proceeded smoothly overnight leading to 34. Subsequent condensation with [(2Z)-2-[(3S,5R)-3,5-bis(tert-butyldimethylsilanyloxy)-2-methylenecyclohexylidene]ethyl]diphenylphosphine oxide (28) gave 35. One deprotection step with tetrabutylammonium fluoride liberated all five protected hydroxyl groups and, after chromatographic purification, compound 2 was obtained.

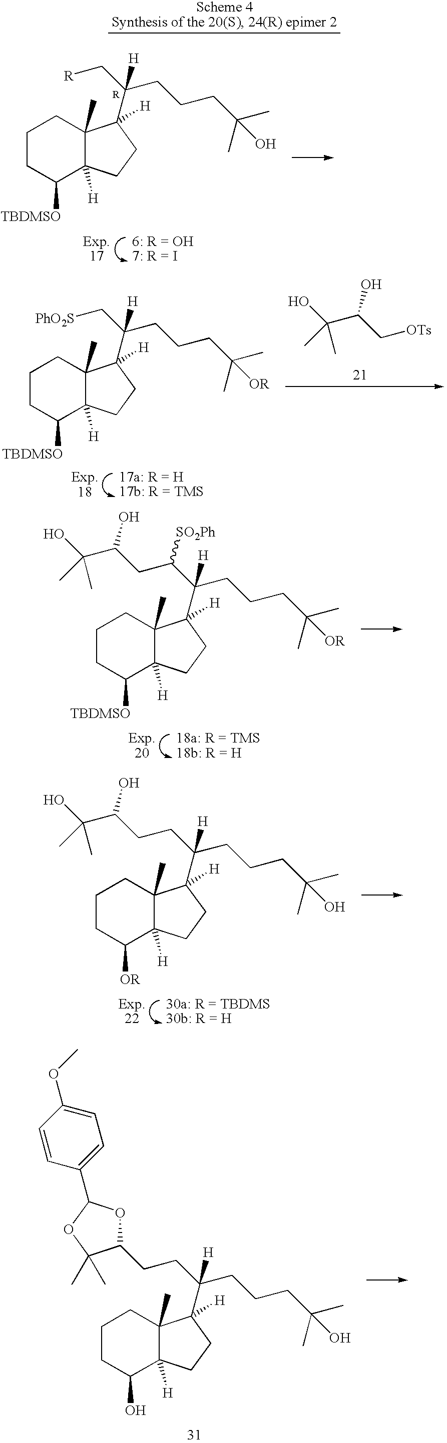

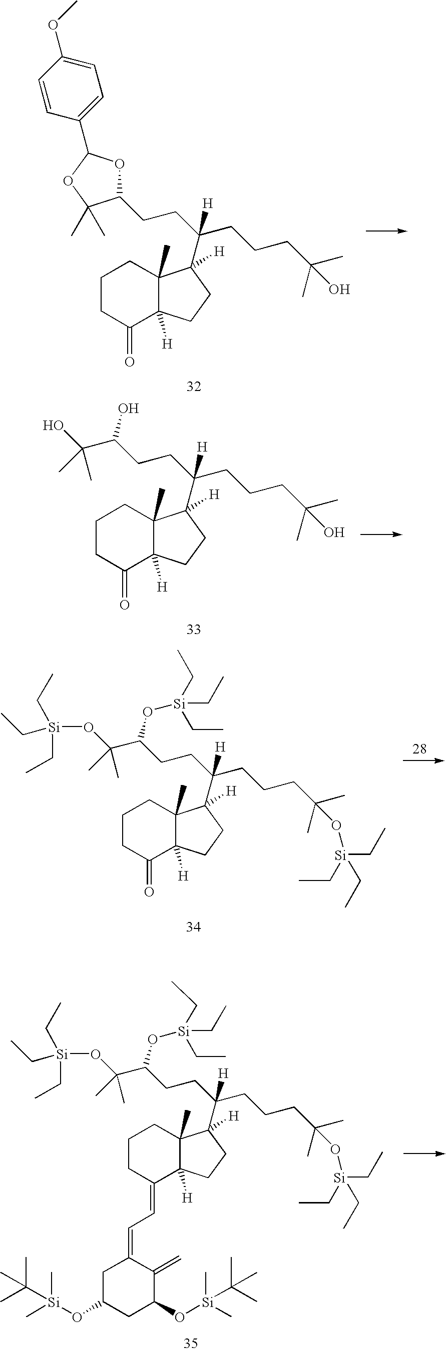

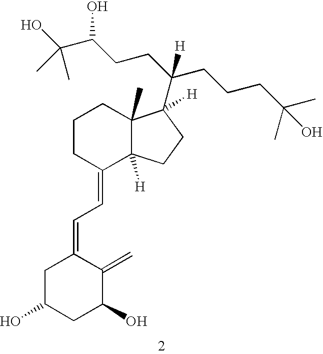

### Synthesis of 1,25-Dihydroxy-21-(2R,3-dihydroxy-3-meth 1-butyl)-20S-19-nor-cholecalciferol (38)

As shown in Schemes 4 and 5, the synthesis of 37 was identical to 2 up through 34. However, instead of reacting 34 with 28, 34 was reacted with [2-[(3R,5R)-3,5-bis(tert-butyldimethylsilanyloxy)cyclohexylidene]ethyl]diphenylphosphine oxide (36) to yield 37 which deprotected with tetrabutyl ammonium fluoride to yield 38.

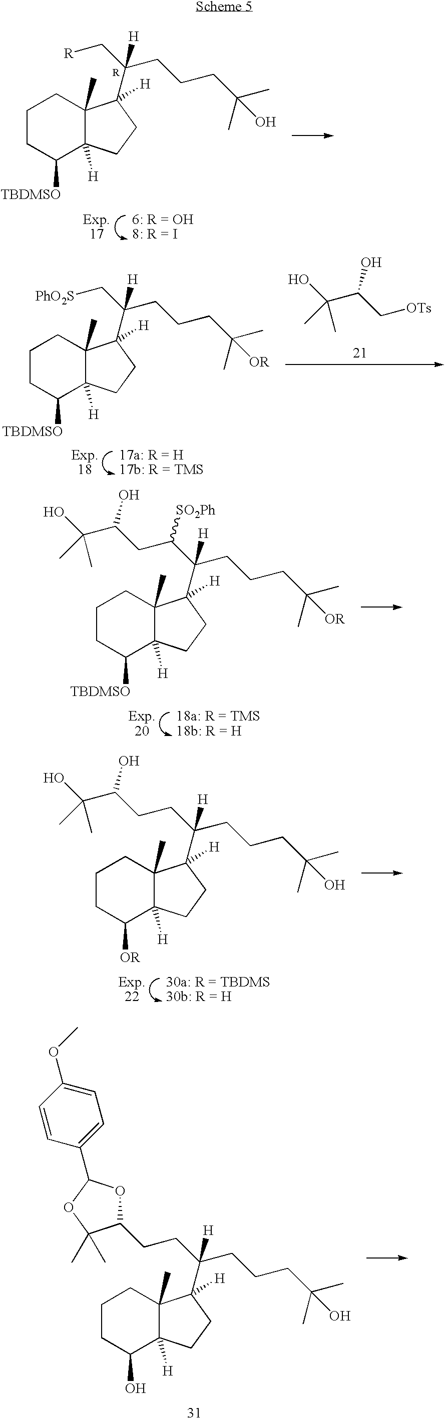

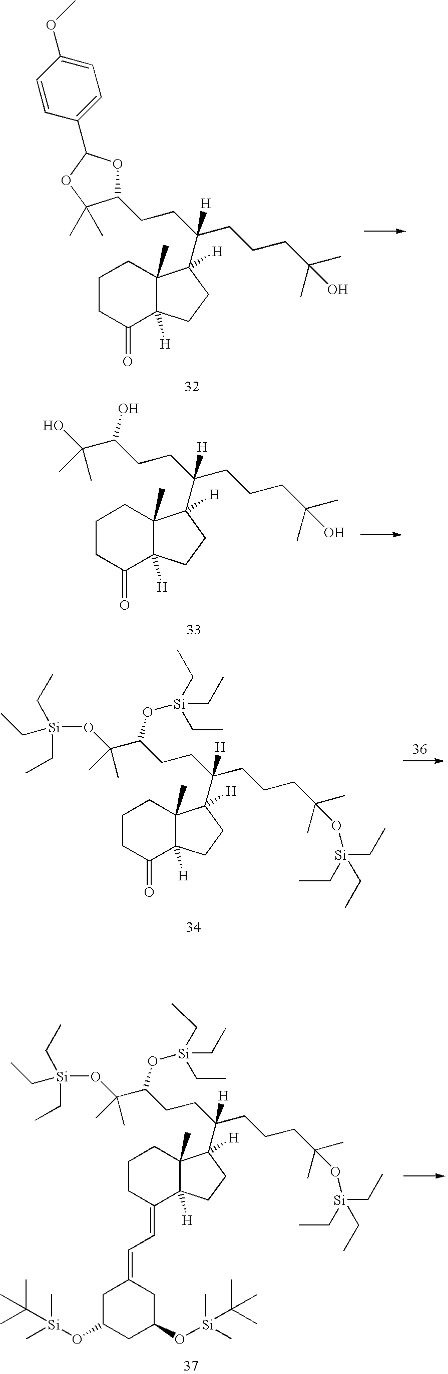

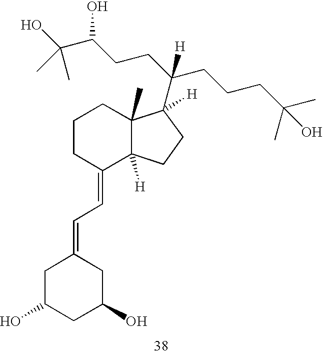

### B. Synthesis of 24-Keto Gemini Vitamin D₃ Compounds

Scheme 6 below graphically depicts the reaction steps for the synthesis of the 24-keto Gemini vitamin D₃ compounds of the invention, namely 1,25-dihydroxy-20S-21 (3-hydroxy-3-methyl-butyl)-24-keto-19-nor-cholecalciferol (12) and 1,25-dihydroxy-20S-21(3-hydroxy-3-methyl-butyl)-24-keto-cholecalciferol (14).

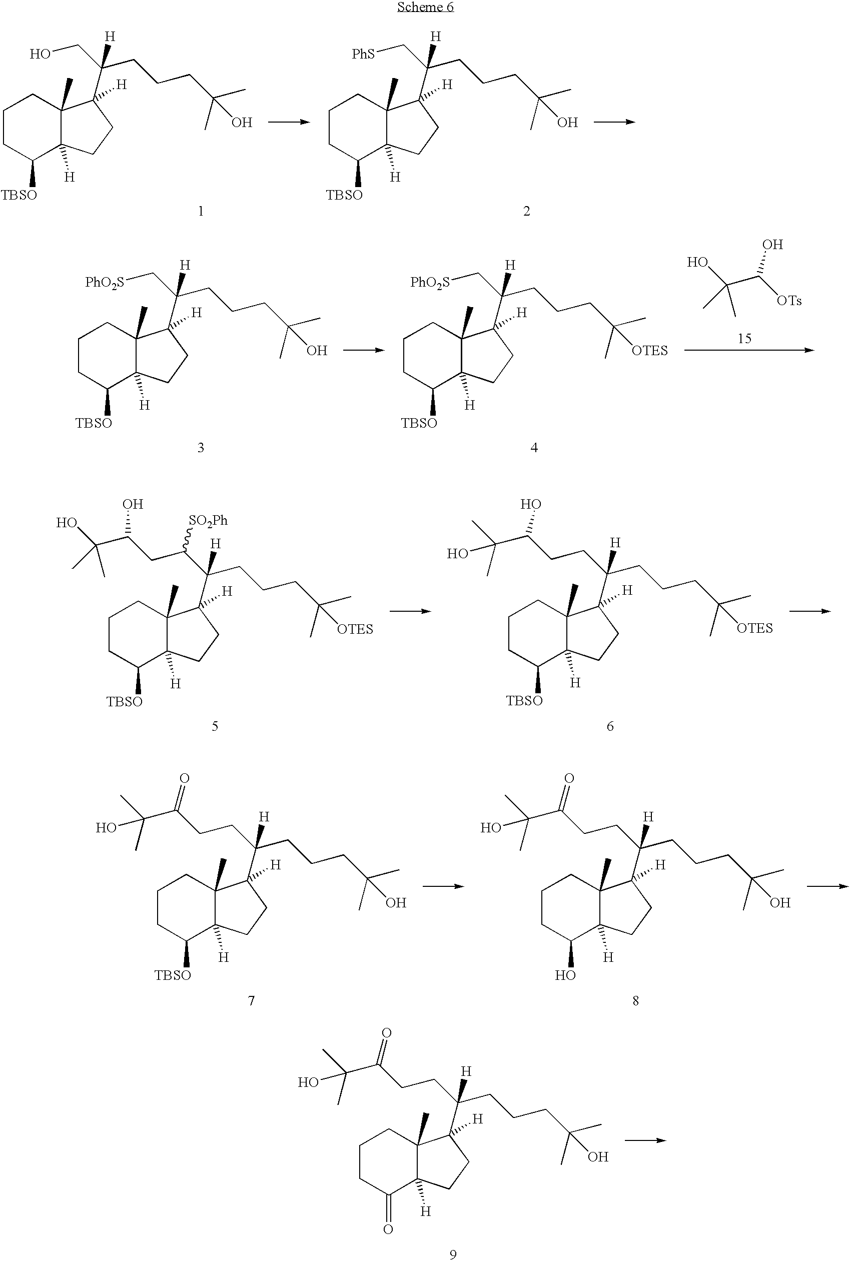

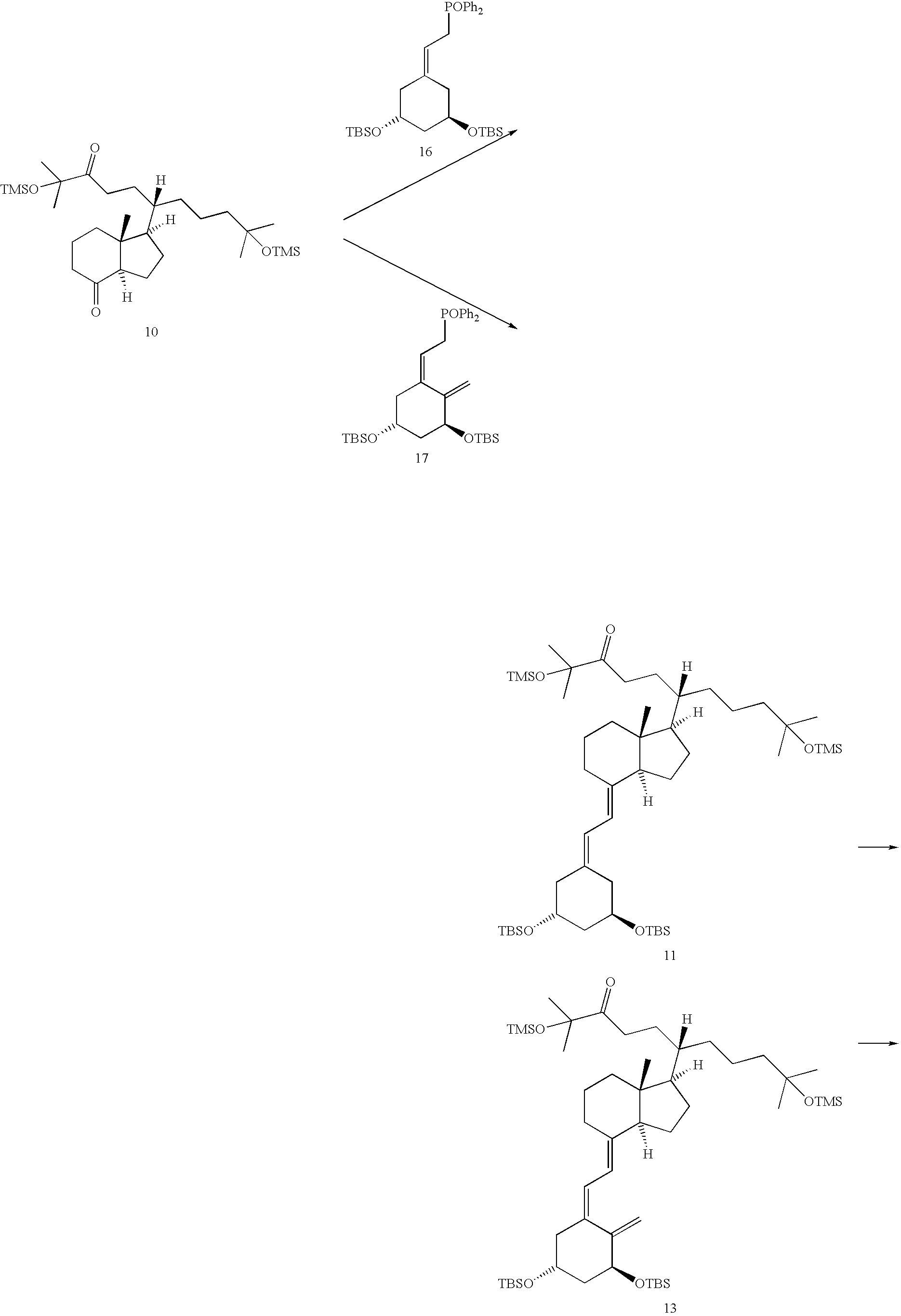

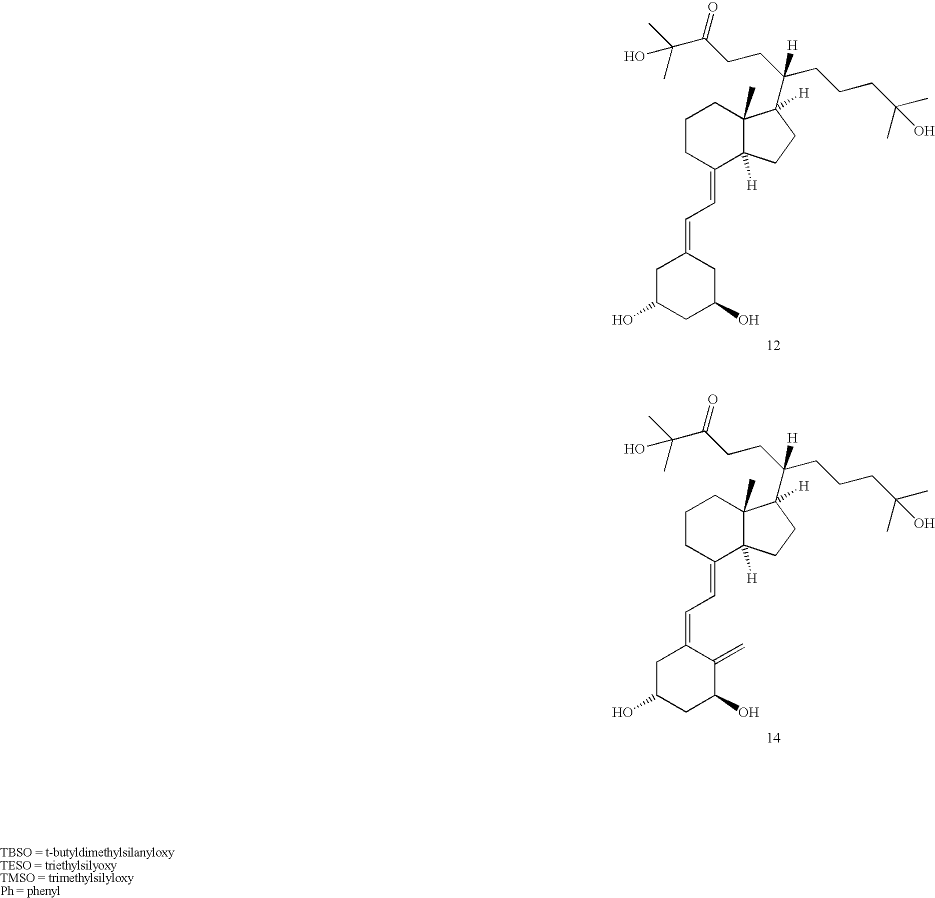

As is evident from Scheme 6, the syntheses of final products 12 and 14 are identical through intermediate 10. For 12, 10 is reacted with [2-[(3R,5R)-3,5 bis(tertbutyldimethylsilanyloxy)-cyclohexylidene]ethyl]diphenylphosphine oxide (16) to yield 11 followed by deprotection with tetrabutylammonium fluoride to yield 12. To obtain 14, 10 is reacted with 3,5-bis(tert-butyldimethylsilanyloxy)-2-methylenecyclohexylidene]ethyl]-diphenylphosphine oxide (17) to yield 13 followed by deprotection with tetrabutylammonium fluoride to yield 14.

### C. Synthesis of-1,25-Dihydroxy-21(3-hydroxy-3-trifluoromethyl-4-trifluoro-butynyl-26,27-hexadeutero-19-nor-20S-cholecalciferol (39) and 1,25-Dihydroxy-21(3-hydroxy-3-trifluoromethyl-4-trifluoro-butynyl)-26,27-hexadeutero-20S-cholecalciferol (40)

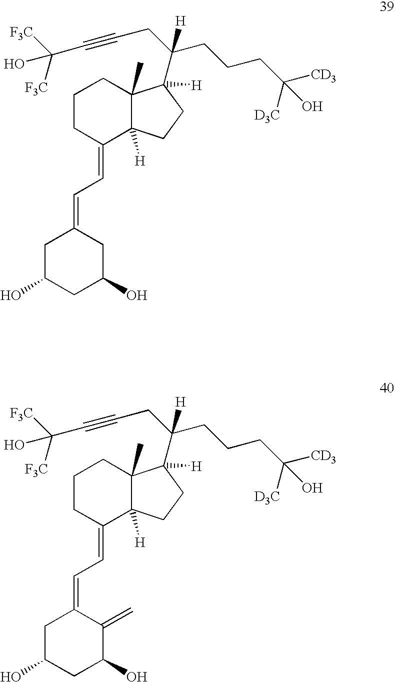

Reaction schemes 7, 8 and 9 below graphically depict the reaction steps for the synthesis of the non-deuterated compounds 6b and 6a (Maehr, H. and Uskokovic, M., Eur. J. Org. Chem. 1703-1713 (2004)) that correspond to the deuteromethyl Gemini vitamin D₃ compounds of the invention, namely 1,25-Dihydroxy-21(3-hydroxy-3-trifluoromethyl-4-trifluoro-butynyl)-26,27-hexadeutero-19-nor-20S-cholecalciferol (39) and 1,25-Dihydroxy-21(3-hydroxy-3-trifluoromethyl-4-trifluoro-butynyl)-26,27-hexadeutero-20S-cholecalciferol (40), respectively.

The synthesis of compounds 39 and 40 is virtually identical to the literature synthesis of non-deuterated compounds 6b and 6a described in the preceding paragraph. The syntheses of the hexadeutero compounds of the invention and the non-deuterated compounds of the literature are different in only one step—the conversion of intermediate 11 to intermediate 12 shown in Scheme 7. In converting intermediate 11 to intermediate 12, methyl-d3-magnesium bromide was used to make the hexadeutero compounds 39 and 40, instead of the methylmagnesium bromide that was used in the literature synthesis to make the corresponding non-deuterated compounds.

In Scheme 9 below, the non-deuterated final product 6b corresponds to 1,25-Dihydroxy-21(3-hydroxy-3-trifluoromethyl-4-trifluoro-butynyl)-26,27-hexadeutero-19-nor-20S-cholecalciferol (39). In scheme 9 below, the non-deuterated final product 6a corresponds to Dihydroxy-21(3-hydroxy-3-trifluoromethyl-4-trifluoro-butynyl)-26,27-hexadeutero-20S-cholecalciferol (40).

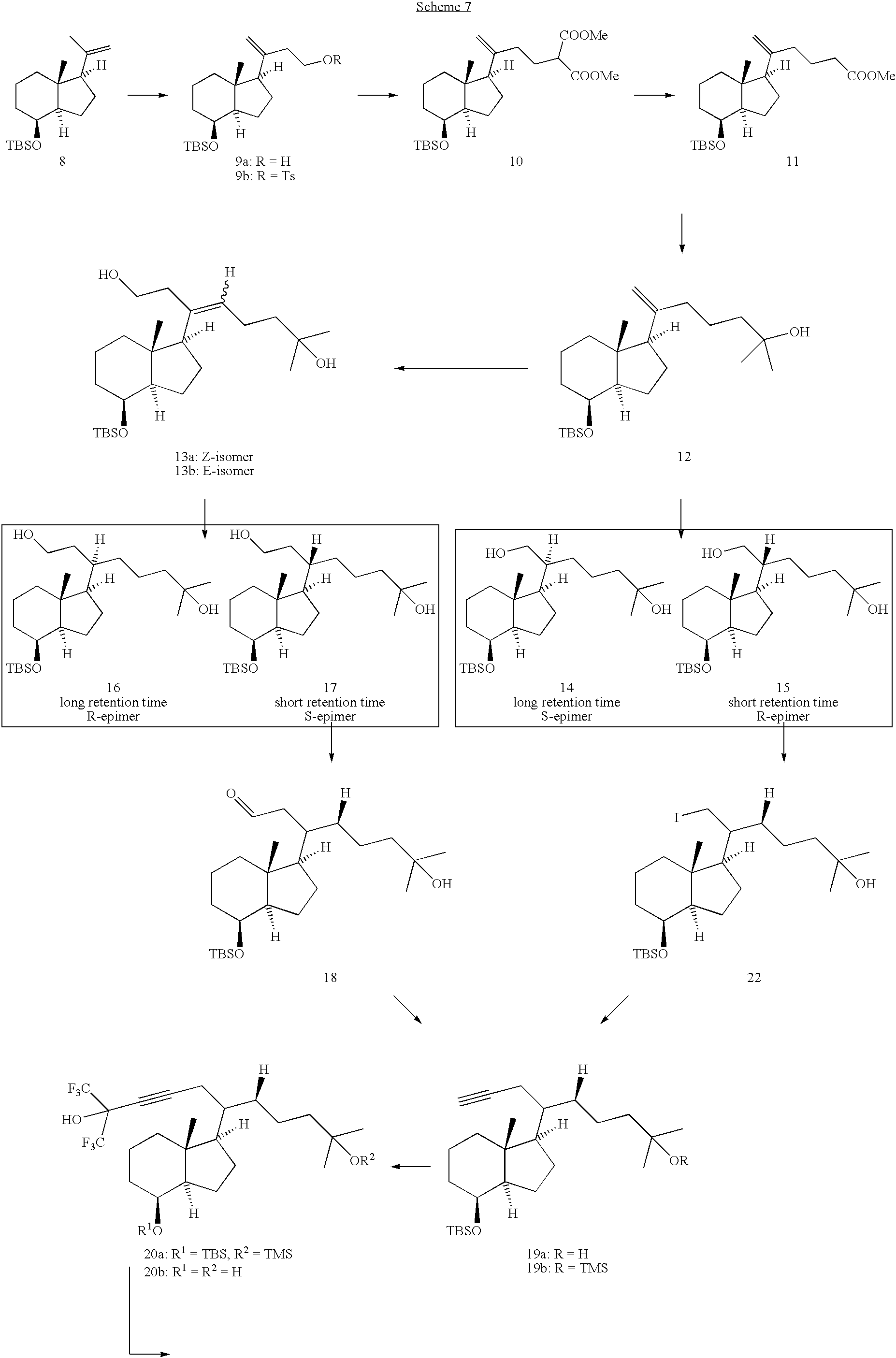

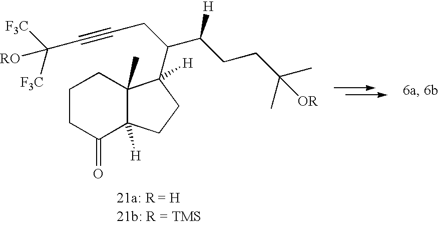

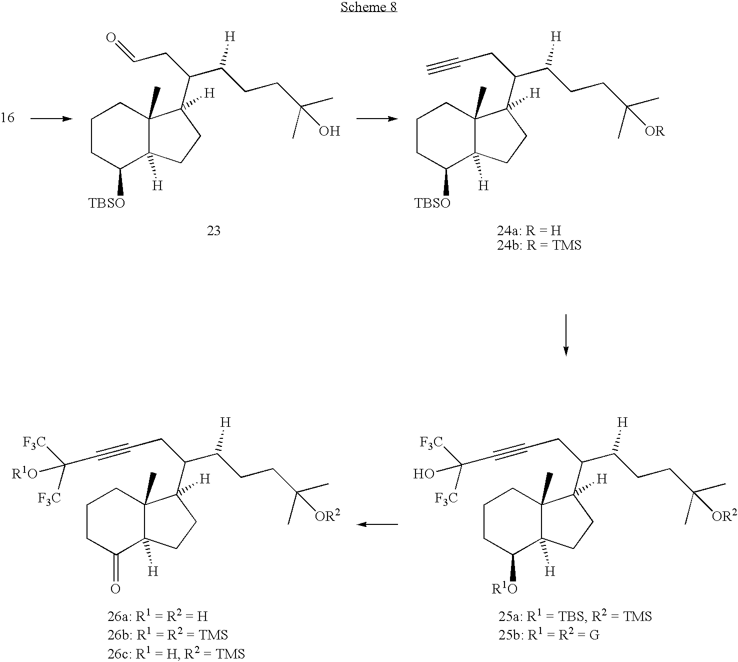

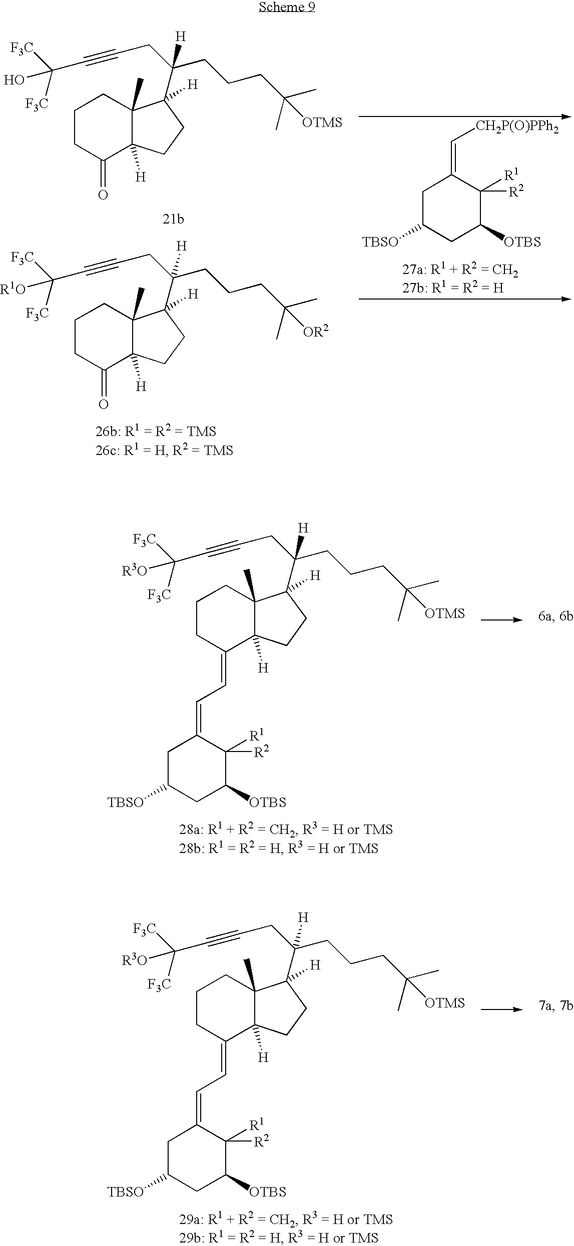

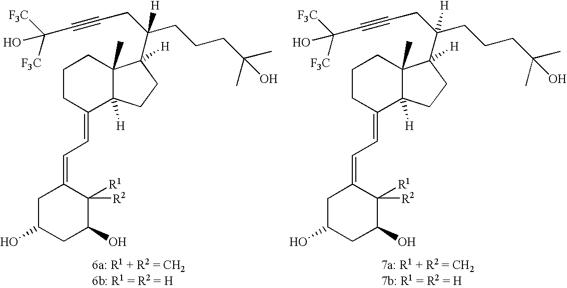

Chiral synthesis can result in products of high stereoisomer purity. However, in some cases, the stereoisomer purity of the product is not sufficiently high. The skilled artisan will appreciate that the separation methods described herein can be used to further enhance the stereoisomer purity of the vitamin D₃-epimer obtained by chiral synthesis.

Any novel syntheses, described herein, of the compounds of the invention, and of intermediates thereof, are also intended to be included within the scope of the present invention.

### EXEMPLIFICATION OF THE INVENTION

The invention is further illustrated by the following examples which should in no way should be construed as being further limiting.

### Synthesis of Compounds of the Invention

### EXPERIMENTAL

All operations involving vitamin D₃ analogs were conducted in amber-colored glassware in a nitrogen atmosphere. Tetrahydrofuran was distilled from sodium-benzophenone ketyl just prior to its use and solutions of solutes were dried with sodium sulfate. Melting points were determined on a Thomas-Hoover capillary apparatus and are uncorrected. Optical rotations were measured at 25° C. ¹H NMR spectra were recorded at 400 MHz in CDCl₃ unless indicated otherwise. TLC was carried out on silica gel plates (Merck PF-254) with visualization under short-wavelength UV light or by spraying the plates with 10% phosphomolybdic acid in methanol followed by heating. Flash chromatography was carried out on 40-65 μm mesh silica gel. Preparative HPLC was performed on a 5×50 cm column and 15-30 μm mesh silica gel at a flow rate of 100 mL/min.

### Example 1

### Synthesis of 1,25-Dihydroxy-21-(2R,3-dihydroxy-3-methyl-butyl)-20R-Cholecalciferol (3)

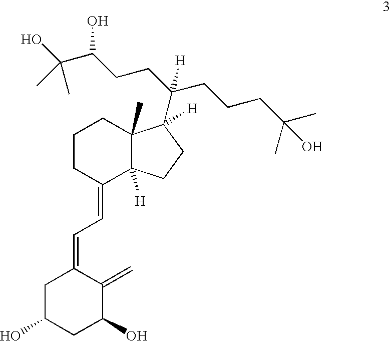

### [1R,3aR,4S,7aR]-2(R)-[4-(1,1-dimethylethyl)dimethyl-silanyloxy)-7a-methyl-octahydro-inden-1-yl]-6-methyl-heptane-1,6-diol (6) and [1R,3aR,4S,7aR]-2(S)-[4-(1,1-dimethylethyl)dimethyl-silanyloxy)-7a-methyl-octahydro-inden-1-yl]-6-methyl-heptane-1,6-diol (7)

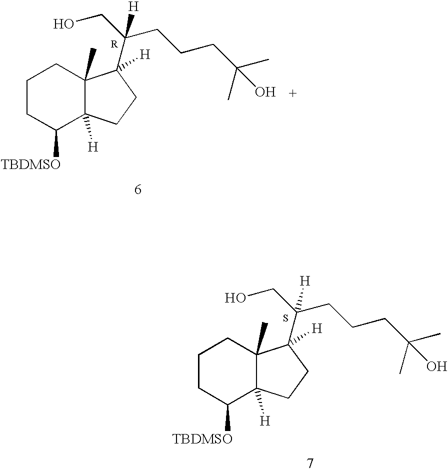

A solution of the alkenol 5 in tetrahydrofuran (9 mL) was cooled in an ice bath and a 1 M solution of borane-THF in tetrahydrofuran (17 mL) was added dropwise in an originally effervescent reaction. The solution was stirred overnight at room temperature, re-cooled in an ice bath water (17 mL) was added dropwise followed by sodium percarbonate (7.10 g, 68 mmol). The mixture was immersed into a 50° C. bath and stirred for 70 min to generate a solution. The two-phase system was allowed to cool then equilibrated with 1:1 ethyl acetate-hexane (170 mL). The organic layer was washed with water (2×25 mL) then brine (20 mL), dried and evaporated to leave a colorless oil (2.76 g). This material was passed through a short flash column using 1:1 ethyl acetate-hexane and silica gel G. The effluent, obtained after exhaustive elution, was evaporated, taken up in ethyl acetate, filtered and chromatographed on the 2×18″ 15-20μ silica YMC HPLC column using 2:1 ethyl acetate-hexane as mobile phase and running at 100 mL/min. Isomer 6 emerged at an effluent maximum of 2.9 L, colorless oil, 1.3114 g, [α]_{D}+45.2° (methanol, c 0.58; ¹H NMR δ-0.002 (3H, s), 0.011 (3H, s), 0.89 (9H, s), 0.93 (3H, s), 1.17 (1H, m), 1.22 (6H, s), 1.25-1.6 (16H, m), 1.68 (1H, m), 1.80 (2H, m), 1.89 (1H, m), 3.66 (1H, dd, J=4.8 and 11 Hz), 3.72 (1H, dd, J=3.3 and 11 Hz), 4.00 (1H, m); LR-ES(−) m/z 412 (M), 411 (M−H); HR-ES(+): calcd for (M+Na) 435.3265, found: 435.3269.

Isomer 7 at was eluted at an effluent maximum of 4.9 L, colorless oil, 0.8562 g that crystallized upon prolonged standing: mp 102-3°, [α]_{D}+25.2° (methanol, c 0.49); ¹H NMR δ-0.005 (3H, s), 0.009 (3H, s), 0.89 (9H, s), 0.93 (3H, s), 1.16 (1H, m), 1.22 (6H, s), 1.3-1.5, (14H, m), 1.57 (2H, m), 1.67 (1H, m), 1.80 (2H, m), 1.91 (1H, m), 3.54 (1H, dd, J=4.8 and 11 Hz), 3.72 (1H, dd, J=2.9 and 11 Hz), 4.00 (1H, m);); LR-ES(−) m/z 412 (M), 411 (M−H). Anal. Calcd for C₂₄H₄₈O₃Si: C, 69.84; H, 11.72; found: C, 69.91; H, 11.76.

### [1R,3aR,4S,7aR]-6(R)-[4-(tert-Butyl-dimethyl-silanyloxy)-7a-methyl-octahydro-inden-1-yl]-7-iodo-2-methyl-heptan-2-ol (8)

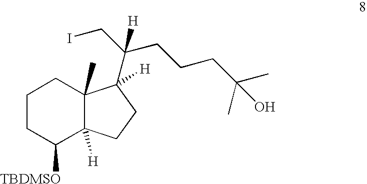

A stirred mixture of triphenylphosphine (0.333 g, 1.27 mmol) and imidazole (0.255 g, 3 mmol) in dichloromethane (3 mL) was cooled in an ice bath and iodine (0.305 g, 1.20 mmol) was added. This mixture was stirred for 10 min then a solution of 6 (0.4537 g, 1.10 mmol) in dichloromethane (3 mL) was added dropwise over a 10 min period. The mixture was stirred in the ice bath for 30 min then at ambient temperature for 2¾ h. TLC (1:1 ethyl acetate-hexane) confirmed absence of educt. A solution of sodium thiosulfate (0.1 g) in water (5 mL) was added, the mixture equilibrated and the organic phase washed with 0.1 N sulfuric acid (10 mL) containing a few drops of brine then with 1:1 water-brine (2×10 mL), once with brine (10 mL) then dried and evaporated. The residue was purified by flash chromatography using 1:9 ethyl acetate-hexane as mobile phase to furnish 8 as a colorless syrup, 0.5637 g, 98%: ¹H NMR δ-0.005 (3H, s), 0.010 (3H, s), 0.89 (9H, s), 0.92 (3H, s), 1.23 (6H, s), 1.1-1.6 (16H, m), 1.68 (1H, m), 1.79 (2H, m), 1.84 (1H, m), 3.37 (11, dd, J=4 and 10 Hz), 3.47 (1H, dd, J=3 and 10 Hz), 4.00 (1H, m); LR-EI(+) m/z-522 (M), 465 (-.C₄H₉), 477 (M−C₄H₉—H₂O); HR-EI(+): calcd for C₂₄H₄₇IO₂Si: 522.2390, found: 522.2394.

### [1R,3aR,4S,7aR]-6(S)-[4-(tert-Butyl-dimethyl-silanyloxy)-7a-methyl-octahydro-inden-1-yl]-2-methyl-non-8-yn-2-ol (14)

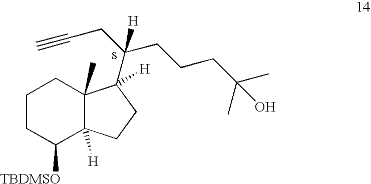

Lithium acetylide DMA complex (0.110 g, 1.19 mmol) was added to a solution of 8 (0.2018 g (0.386 mmol) in dimethyl sulfoxide (1.5 mL) and tetrahydrofuran (0.15 mL). The mixture was stirred overnight. TLC (1:4 ethyl acetate-hexane) showed a mixture of two spots traveling very close together (Rf 0.52 and 0.46). Fractions at the beginning of the eluted band contained pure 5, which is the elimination product of 8, and was produced as the major product. Fractions at the end of the elution band, however, were also homogeneous and gave the desired acetylene 14 upon evaporation. The NMR spectra of 14 and its 6-epimer derived from 12 which served for identification were previously reported.⁹

### [1R,3aR,4S,7aR]-7-Benzenesulfonyl-6(S)-[4-(tert-butyl-dimethyl-silanyloxy)-7a-methyl-octahydro-inden-1-yl]-2-methyl-heptan-2-ol (19a)

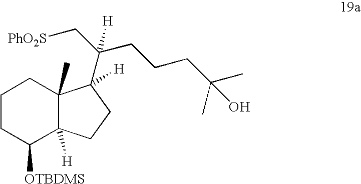

A mixture of 9 (0.94 g, 1.8 mmol), sodium benzenesulfinate (2.18 g, 13 mmol) and N,N-dimethylformamide (31.8 g) was stirred at room temperature for 12 h, then in a 40° C. bath for ca.6 h until all educt was converted as shown by TLC (1:4 ethyl acetate-hexane). The solution was equilibrated with 1:1 ethyl acetate-hexane (120 mL) and 1:1 brine-water (45 mL). The organic layer was washed with water (4×25 mL) brine (10 mL), then dried and evaporated to leave a colorless oil, 1.0317 g. This material was flash-chromatographed using a stepwise gradient (1:9, 1:6, 1:3 ethyl acetate-hexane) to give a colorless oil, 0.930 g, 96%: 300 MHz ¹H NMR δ-0.02 (3H, s), 0.00 (3H, s), 0.87 (9H, s), 0.88 (3H, s), 1.12 (1H, m), 1.20 (6H, s), 1.2-1.8 (18H, m), 1.81 (1H, m), 3.09 (2H, m), 3.97 (1H, brs), 7.59 (3H, m), 7.91 2H, m).

### [1R,3aR,4S,7aR]-1-(1(S)-Benzenesulfonylmethyl-5-methyl-5-trimethylsilanyloxy-hexyl)-4-(tert-butyl-dimethyl-silanyloxy)-7a-methyl-octahydro-indene (19b)

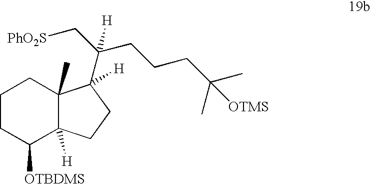

1-(Trimethylsilyl)imidazole (1 mL) was added to a solution of 19a (0.8 g) in cyclohexane (10 mL) and stirred overnight then flash-chromatographed using a stepwise gradient of hexane, 1:39 and 1:19 ethyl acetate-hexane. The elution was monitored by TLC (1:4 ethyl acetate-hexane) leading to 19b as a colorless syrup, 0.7915 g: 300 MHz ¹H NMR 8 0.00 (3H, s), 0.02 (3H, s), 0.12 (9H, s), 0.90 (12H, s, t-butyl+7a-Me), 1.16 (1H, m), 1.20 (6H, s), 1.2-1.6 (15H, m), 1.66-1.86 (3H, m), 3.10 (2H, m), 4.00 (1H, brs), 7.56-7.70 (3H, m), 7.93 (2H, m).

### [1R,3aR,4S,7aR]-6(R)-[4-(tert-Butyl-dimethyl-silanyloxy)-7a-methyl-octahydro-inden-1-yl]-2,10-dimethyl-undecane-2,3(R),10-triol (22b)

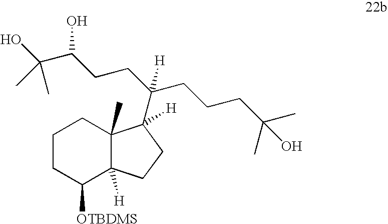

A solution of 19b (0.7513 g, 1.23 mmol) and diol 21 (0.508 g, 1.85 mmol) in tetrahydrofuran (28 mL) was cooled to −35° C. then 2.5 M butyllithium in hexane (2.75 mL) was added dropwise. The temperature was allowed to rise to −20° C. and maintained at that temperature for 6 h or until the educt was consumed. Reaction progress was monitored by TLC (1:4 ethyl acetate-hexane) exhibiting the educt (Rf 0.71) and the two epimeric diols 20 (Rf 0.09 and 0.12). Toward the end of the reaction period the temperature was increased briefly to 0° C., lowered again to −10, then saturated ammonium chloride (25 mL) was added followed by ethyl acetate (50 mL) and enough water to dissolve the precipitated salts. The resulting aqueous phase was extracted with ethyl acetate (15 mL). The combined extracts were washed with brine (15 mL), dried and evaporated. The resulting syrup was flash-chromatographed using a stepwise gradient of 1:9, 1:6, 1:4 and 1:1 ethyl acetate-hexane to give 20 as a colorless syrup, 0.8586 g. This material was dissolved in a mixture of tetrahydrofuran (30 mL) and methanol (18 mL), then 5% sodium amalgam (20g) was added. The reductive de-sulfonylation was complete after stirring of the mixture for 14 h. Progress of the reaction was monitored by TLC (1:1 ethyl acetate-hexane) which showed the disappearance of the epimeric 20 (Rf 0.63 and 0.74) and the generation of 22a (Rf 0.79) and the partially de-silylated analog 22b (Rf 0.16). The mixture was diluted with methanol (20 mL), stirred for 3 min, then ice (20g) was added, stirred for 2 min and the supernatant decanted into a mixture containing saturated ammonium chloride (50 mL). The residue was repeatedly washed with small amounts of tetrahydrofuran that was also added to the salt solution, which was then equilibrated with ethyl acetate (80 mL). The aqueous layer was re-extracted once with ethyl acetate (20 mL), the combined extracts were washed with brine (10 mL) then dried and evaporated. The resulting colorless oil containing both 22a and 22b was dissolved in 10 mL of a 1 N oxalic acid solution in methanol (prepared from the dihydrate) effecting the selective hydrolysis of the trimethylsilyl ether within minutes. Calcium carbonate (1g) was added and the suspension stirred overnight, then filtered. The solution was evaporated and the resulting residue flash-chromatographed using a stepwise gradient of 1:4, 1:2, 1:1 and 2:1 ethyl acetate-hexane giving a residue of the triol 22b that crystallized in very fine branching needles from acetonitrile, 0.45 g: mp 94-95° C., [α]_{D}+44.1° (methanol, c 0.37); 400 MHz ¹H NMR δ-0.005 (3H, s), 0.007 (3H, s), 0.89 (9H, s), 0.92 (3H, s), 1.15 (1H, m), 1.16 (3H, s), 1.21 (9H, s), 1.2-1.6 (19H, m), 1.67 (1H, m), 1.79 (2H, m), 1.90 (2H, m), 2.06 (1H, m), 3.31 (1H, brd, J=10 Hz), 4.00 (1H, brs), LR-ES(−) m/z: 533 (M+Cl), 497 (1-H); HR-ES(+): Calcd for C₂₉H₅₈O₄Si+Na: 521.3996, found: 521.4003. Anal Calcd for C₂₉H₅₈O₄Si: C, 69.82; H, 11.72; found: C, 69.97; H, 11.65.

### [1R,3aR,4S,7aR]-6(R)-(4-Hydroxy-7a-methyl-octahydro-inden-1-yl)-2,10-dimethyl-undecane-2,3(R),10-triol (22c)

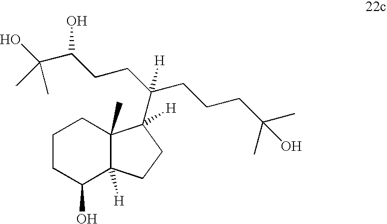

A stirred solution of the triol 22b (0.4626 g, 0.927 mmol) in acetonitrile (10 mL) and dioxane (0.7 mL) was cooled to 10° C. and a fluorosilicic acid solution (2 mL) was added dropwise. The cooling bath was removed, the 2-phase system further diluted with acetonitrile (2 mL) then stirred at room temperature for 3¼ h. The disappearance of educt was monitored by TLC (ethyl acetate). The mixture was equilibrated with water (10 mL) and ethyl acetate (30 mL). The aqueous phase was re-extracted with ethyl acetate (2×20 mL), the combined extracts were washed with water (5 mL) and brine (10 mL), then 1:1 brine-saturated sodium hydrogen carbonate solution and dried. The residue was purified by flash-chromatography using a step-wise gradient from 1:1 to 2:1 ethyl acetate-hexane and neat ethyl acetate to give a residue that was taken up in 1:1 dichloromethane-hexane, filtered and evaporated to furnish amorphous solids, 0.3039 g (85%): [α]_{D}+42.6° (methanol, c 0.48); ¹H NMR (DMSO-d₆): δ 0.87 (3H, s), 0.97 (3H, s), 1.02 (3H, s), 1.04 (6H, s), 1.1-1.4 (18H, m), 1.5-1.8 (4H, m), 1.84 (1H, m), 2.99 (1H, dd, J=6 and 10 Hz), 3.87 (1H, brs), 4.02 (1H, s, OH), 4.05 (1H, s, OH), 4.16 (1H, d, OH, J=3.6 Hz), 4.20 (1H, d, OH, J=6.4 Hz); LR-ES(+): m/z 384 (M), 383 (M−H); HR-ES(+): Calcd for (M+Na) 407.3132, found: 407.3134.

### [1R,3aR,4S,7aR]-1-{5-Hydroxy-5-methyl-1(R)-[2-(2,2,5,5-tetramethyl-[1,3]dioxolan-4(R)-yl)-ethyl]-hexyl}-7a-methyl-octahydro-inden-4-ol (23a)

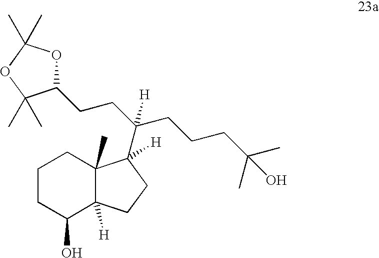

A solution of the tetraol 22b (0.2966 g, 0.771 mmol) and pyridinium tosylate (100 mg) in acetone (8 mL) and 2,2-dimethoxypropane (8 mL) was kept at room temperature for 12 h. TLC analysis (ethyl acetate) showed the absence of educt (Rf 0.21) and two new spots with Rf 0.82 and 0.71, the former the expected 23a and the latter assumed to be the methylacetal 23. The reaction mixture was diluted with water (5 mL) and stirred for 10 min. At that time only the spot with higher Rf value was observed. The mixture was neutralized with sodium hydrogen carbonate (0.5 g) then equilibrated with ethyl acetate (50 mL) and brine (5 mL). The organic layer was washed with water (5 mL) and brine (5 mL) then dried and evaporated to leave a sticky residue (0.324 g) that was used directly in the next step: 300 MHz ¹H NMR: δ 0.94 (3H, s), 1.10 (3H, s), 1.20 (1H, m), 1.22 (6H, s), 1.25 (3H, s), 1.34 (3H, s), 1.41 (3H, s), 1.2-1.65 (20H, m), 1.78-1.86 (3H, m), 1.93 (1H, m), 3.62 (1H, dd, J=4.6 and 8.3 Hz), 4.08 (1H, brs).

### [1R,3aR,4S,7aR]-Acetic acid 1-{5-hydroxy-5-methyl-1(R)-[2-(2,2,5,5-tetramethyl-[1,3]dioxolan-4(R)-yl)-ethyl]-hexyl}-7a-methyl-octahydro-inden-4-yl ester (23b)

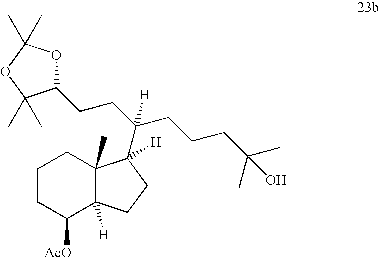

The residue obtained above was dissolved in pyridine (6.9 g) and further diluted with acetic anhydride (3.41 g). The mixture was allowed to stand at room temperature for 24 h, then in a 35° C. bath for ca. 10 h until the educt was no longer detectable (TLC, ethyl acetate). The mixture was diluted with toluene and evaporated. The residue was purified by flash chromatography (1:4 ethyl acetate-hexane) to give 23b as colorless syrup, 0.3452 g, 97%: ¹H NMR: δ 0.89 (3H, s), 1.10 (3H, s), 1.20 (1H, m), 1.22 (6H, s), 1.25 (3H, s), 1.33 (3H, s), 1.41 (3H, s), 1.25-1.6 (19H, m), 1.72 (1-, m), 1.82 (2H, m), 1.95 (1H, m), 2.05 (3H, s), 3.63 (1H, dd, J=4.4 and 8.4 Hz), 5.15 (1H, brs); LR-FAB(+) m/z 467 (+M), 465 (M−H), 451 (M−Me).

### [1R,3aR,4S,7aR]-Acetic acid 1-[4(R),5-dihydroxy-1(R)-(4-hydroxy-4-methyl-pentyl)-5-methyl-hexyl]-7a-methyl-octahydro-inden-4-yl ester (24)

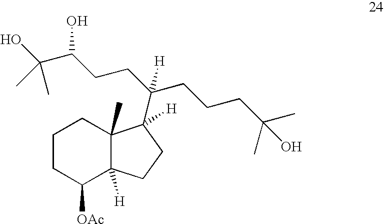

A solution of 23b (0.334 g, 0.716 mmol) in 80% acetic acid (2 mL) was kept in a 68° C. bath. TLC (ethyl acetate, Rf 0.33) monitored the progress of the hydrolysis. The educt was no longer detectable after 2.5 h. The mixture was evaporated then co-evaporated with a small amount of toluene to leave a colorless film (0.303 g) that was used directly in the next step: 300 MHz ¹H NMR: δ 0.89 (3H, s), 1.17 (3H, s), 1.22 (6H, s), 1.56 (3H, s), 1.1-1.6 (2H, m), 1.6-2.0 (5H, m), 2.04 (3H, s), 3.32 (H, brd, J=10 Hz), 5.15 (1H, brs).

### [1R,3aR,4S,7aR]-Acetic acid 1-[4(R)-[dimethyl-(1,1,2-trimethyl-propyl)-silanyloxy]-5-hydroxy-1(R)-(4-hydroxy-4-methyl-pentyl)-5-methyl-hexyl]-7a-methyl-octahydro-inden-4-yl ester (25a)

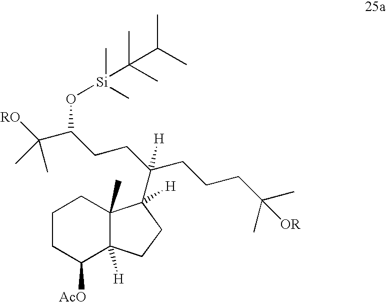

A solution of the triol 24 (0.30 g), imidazole (0.68 g, 10 mmol) and dimethylthexylsilyl chloride (1.34 g, 7.5 mmol) in N,N-dimethylformamide (6g) was kept at room temperature. After 48 h 4-(N,N-dimethylamino)pyridine (5 mg) was added and the mixture stirred for an additional 24 h. Reaction progress was monitored by TLC (ethyl acetate; 24, Rf 0.83; 25a, Rf 0.38). The mixture was diluted with water (2 mL), stirred for 10 min then distributed between ethyl acetate (45 mL) and water (20 mL). The aqueous layer was extracted once with ethyl acetate (10 mL). The combined organic phases were washed with water (4×12 mL) and brine (8 mL) then dried and evaporated. The residual oil was purified by flash-chromatography using a stepwise gradient of 1:9 and 1:4 ethyl acetate-hexane to give 25a as colorless syrup. A small amount of unreacted educt (80 mg) was eluted with ethyl acetate. The syrupy 25a was used directly in the next step: 400 MHz ¹H NMR: δ 0.13 (3H, s), 0.14 (3H, s), 0.87 (6H, s), 0.91 (9H, m), 1.10 (1H, m), 1.14 (3H, s), 1.15 (3H, s), 1.21 (6H, s), 1.1-1.6 (19H, m), 1.6-1.9 (5H, m), 1.94 (1H, brd, J=12.8 Hz), 2.05 (3H, s), 3.38 (1H, brs), 5.15 (1H, brs).

### [1R,3aR,4S,7aR]-Acetic acid 1-[4(R)-[dimethyl-(1,1,2-trimethyl-propyl)-silanyloxy]-5-methyl-1(R)-(4-methyl-4-trimethysilanyloxy-pentyl)-5-trimethylsilanyloxy-hexyl]-7a-methyl-octahydro-inden-4-yl ester (25b)

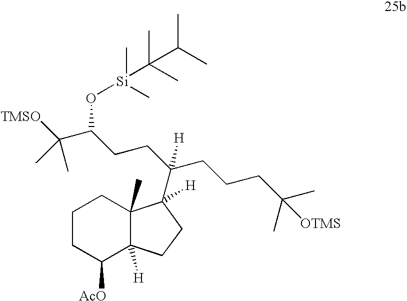

1-(Trimethylsilyl)imidazole (0.90 mL, 6.1 mmol) was added to a solution of 25a (0.2929 mg) in cyclohexane (6 mL) and stirred for 12 h, then flash-chromatographed (1:79 ethyl acetate-hexane) to yield 25b as colorless syrup (0.3372 g). The elution was monitored by TLC (1:4 ethyl acetate-hexane) leading to 19b as a colorless syrup, 0.7915 g: ¹H NMR δ: 0.074 (3H, s), 0.096 (3H, s), 0.103 (9H, s), 0.106 (9H, s), 0.82 (1H, m), 0.83 (6H, s), 0.88 (9H, m), 1.32 (3H, s), 1.20 (9H, s), 1.15-1.6 (17H, m), 1.6-1.9 (5H, m), 1.97 (1H, brd, J=12.8 Hz), 2.05 (3H, s), 3.27 (1H, m), 5.15 (11F, brs); LR-FAB(+) m/z: 712 (M), 711 (M−H), 697 (M−Me), 653 (M−AcO), 627 (M−C₆H₁₃).

### [1R,3aR,4S,7aR]-1-[4(R)-[Dimethyl-(1,1,2-.trimethyl-propyl)-silanyloxy]-5-methyl-1(R)-(4-methyl-4-trimethylsilanyloxy-pentyl)-5-trimethylsilanyloxy-hexyl]-7a-methyl-octahydro-inden-4-ol (26)

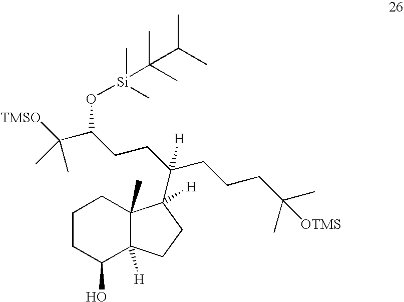

A stirred solution of 25b (0.335 mg, 0.47 mmol) in tetrahydrofuran (15 mL) was cooled in an ice-bath and a 1 M solution of lithium aluminum hydride in tetrahydrofuran (2 mL) was added dropwise. TLC (1:9 ethyl acetate-hexane) showed complete conversion 25b (Rf 0.61) to 26 (Rf 0.29) after 1.5 h. A 2 M sodium hydroxide solution (14 drops) was added, followed by water (0.5 mL) and ethyl acetate (30 mL). A small amount of Celite was added and, after stirring for 15 min, the liquid layer was filtered off. The solid residue was rinsed repeatedly with ethyl acetate and the combined liquid phases evaporated to leave a colorless syrup, that was taken up in hexane, filtered and evaporated to yield 26 (0.335 g) that was used without further purification: ¹H NMR 3: 0.075 (3H, s), 0.10 (2H, brs), 0.82 (1H, m), 0.84 (6H, s), 0.89 (6H, m), 0.93 (3H, s), 1.13 (3H, s), 1.20 (9, s), 1.2-1.6 (16H, m), 1.6-1.7 (2H, m), 1.82 (3H, m), 1.95 (1H, brd, J=12.4 Hz), 3.27 (1H, m), 4.08 (114, brs); LR-FAB(+) m/z: 585 (M−C₆H₁₃),481 (M−TMSO); HR-ES(+) m/z: Calcd for C₃₇H₇₉O₄Si₃+Na: 693.5100 found: 693.5100.

### [1R,3aR,7aR]-1-[4(R)-[Dimethyl-1,1,2-trimethyl-propyl)-silanyloxy]-5-methyl-1(R)-(4-methyl-4-trimethylsilanyloxy-pentyl)-trimethylsilanyloxy-hexyl]-7a-methyl-octahydro-inden-4-one (27)

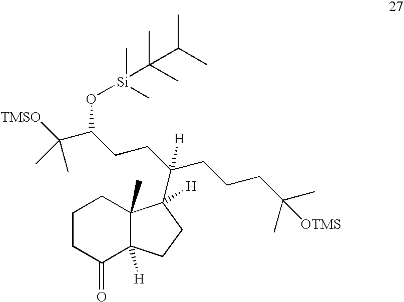

Celite (0.6 g) was added to a stirred solution of 26 (0.310g, 0.462 mmol) in dichloromethane (14 mL) followed by pyridinium dichromate (0.700 g, 1.86 mmol). The conversion of 26 (Rf 0.54) to the ketone 27 (Rf 0.76) was followed by TLC (1:4 ethyl acetate-hexane). The mixture was diluted with cyclohexane after 4.5 h then filtered trough a layer of silica gel. Filtrate and ether washes were combined and evaporated. The residue was flash-chromatographed (1:39 ethyl acetate-hexane) to give 27 as a colorless syrup, 0.2988 g, 96.6%: ¹H NMR δ: 0.078 (3H, s), 0.097 (3H, s), 0.107 (18H, s), 0.64 (3H, s), 0.81 (1H, m), 0.84 (6H, s), 0.89 (6H, m), 1.134 (3H, s), 1.201 (3H, s), 1.207 (3H, s), 1.211 (3H, s), 1.3-1.6 (14H, m), 1.6-1.7 (3H, m), 1.88 (1H, m), 2.04 (2H, m), 2.2-2.32 (2H, m), 2.46 (1H, dd, J=7.5 and 11.5 Hz), 3.28 (1H, m); LR-FAB(+) m/z: 583 (M−C₆H₁₃), 479 M−OTMS); HR-ES(+) m/z: Calcd for C₃₇H₇₆O₄Si₃+Na: 691.4943, found: 691.4949.

### [1R,3aR,7aR,4E]-4-{2(Z)-[3(S),5(R)-Bis-(tert-butyl-dimethyl-silanyloxy)-2-methylene-cyclohexylidene]-ethylidene}-7a-methyl-1-[5-methyl-1(R)-(4-methyl-4-trimethylsilanyloxy-pentyl)-4(R)-[dimethyl-(1,1,2-trimethyl-propyl)-silanyloxy]-5-trimethylsilanyloxy-hexyl]-octahydro-indene (29)

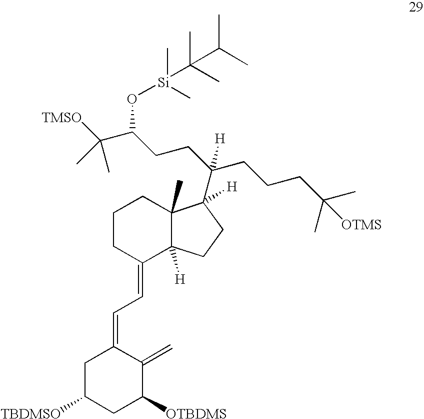

A solution of 2.5-M butyllithium in hexane (0.17 mL) was added to a solution of 28 in tetrahydrofuran (2 mL) at −70° C. to produce a deep cherry-red color of the yield. After 10 min a solution of ketone 27 (0.1415 g, 0.211 mmol) in tetrahydrofuran (2 mL) was added dropwise over a 15 min period. The reaction was quenched after 4 h by the addition of pH 7 phosphate buffer (2 mL). The temperature was allowed to increase to 0° C. then hexane (30 mL) was added. The aqueous layer was re-extracted with hexane (15 mL). The combined extracts were washed with of brine (5 mL), dried and evaporated to give a colorless oil that was purified by flash-chromatography (1:100 ethyl acetate-hexane) to yield 29 as colorless syrup, 0.155 g, 71%: ¹H NMR δ: 0.068 (15H, m), 0.103 (12H, s), 0.107 (9H, s), 0.53 (3H, s), 0.82 (1H, m), 0.84 (6H, s), 0.88 (18H, m), 0.89 (6H, m), 1.14 (3H, m), 1.20 (9H, s), 12-1.9 (22H, m), 1.97 (2H, m), 2.22 (1H, dd, J=7.5 an 13 Hz), 2.45 (1H, brd, J=13 Hz), 2.83 (1H, brd, J=13 Hz), 3.28 (1H, m), 4.20 (1H, m), 4.38 (1H, m), 4.87 (1H, d, J=2 Hz), 5.18 (1H, d, J=2 Hz), 6.02 (1H, d, J=11.4 Hz, 6.24 (1H, d, J=11.4 Hz); LR-FAB(+) m/z 1033 (M+H), 1032 (M), 1031 (M−H), 901 (M−TBDMS).

### Synthesis of 1,25-Dihydroxy-21-(2R,3-dihydroxy-3-methyl-butyl)₂OR-Cholecalciferol (3)

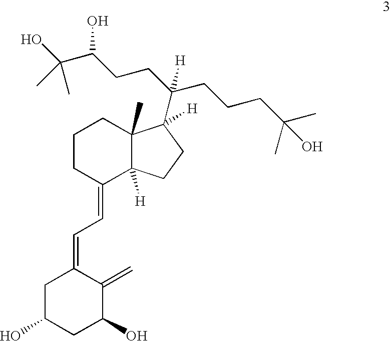

The residue of 29 (0.153 g, 0.148 mmol), as obtained in the previous experiment, was dissolved in a 1 M solution of tetrabutylammonium fluoride (3.5 mL). TLC (ethyl acetate) monitored reaction progress. Thus, the solution was diluted with brine (5 mL) after 24 h, stirred for 5 min then equilibrated with ethyl acetate (35 mL) and water (15 mL). The aqueous layer was re-extracted once with ethyl acetate (15 mL). The combined organic layers were washed with water (5×10 mL), once with brine (5 mL) then dried and evaporated. The residue was purified by flash chromatography using a stepwise gradient of ethyl acetate and 1:100 methanol-ethyl acetate furnishing 3 as colorless, microcrystalline material from methyl formate-pentane, 70 mg, 91%: [α]_{D}+34.3° (methanol, c 0.51); ¹H NMR (DMSO-d₆) δ: 0.051 (3H, s), 0.98 (3H, s), 1.03 (3H, s), 1.05 (6H, s), 1.0-1.6 (17H, m), 1.64 (3H, m), 1.80 (2H, m), 1.90 (1H, d, J=11.7 Hz), 1.97 (1H, dd, J=J=9.8 Hz), 2.16 (1H, dd, J=5.9 and J=13.7 Hz), 2.36 (1H, brd), 2.79 (1H, brd), 3.00 (1H, dd, J=5 and 10 Hz), 3.99 (1H, brs), 4.01 (1H, s, OH), 4.04 (1H, s, OH), 4.54 (1H, OH, d, J=3.9 Hz), 4.76 (1H, brs), 4.87 (1H, OH, d, J=4.9 Hz), 5.22 (1H, brs), 5.99 (1H, d, J=10.7 Hz), 6.19 (1H, d, J=10.7 Hz); LR-ES(+) m/z: 519 (M+H), 518 (M), 517 (M−H), 501 (M−OH); HR-ES(+) calcd for C₃₂H₅₄O₅+Na: 541.3863; found 541.3870; UVₘₐₓ(ε): 213 (13554),241sh (12801), 265 (16029) nm.

### Synthesis of 1,25-Dihydroxy-21(2R,3-dihydroxy-3-methyl-butyl)-20S-Chlolecalciferol (2)

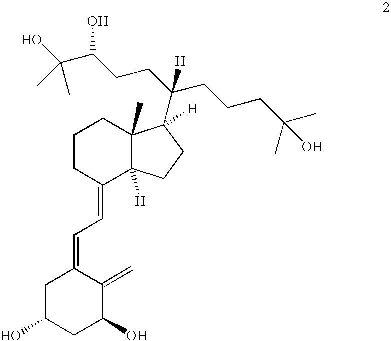

### [1R,3aR,4S,7aR]-7-Benzenesulfonyl-6(R)-[4-(tert-butyl-dimethyl-silanyloxy)-7a-methyl-octahydro-inden-1-yl]-2-methyl-heptan-2-ol (17a)

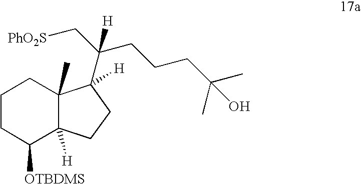

A solution of 8 and sodium benzenesulfinate (0.263 g, 1.6 mmol) in N,N-dimethyl formamide (5 mL) was stirred in a 77° C. bath for 3 h. The solution was equilibrated with 1:1 ethyl acetate-hexane (25 mL) and the organic layer washed with water (5×10 mL), dried and evaporated. The residue was flash-chromatographed with a stepwise gradient of 1:9, 1:4, and 1:3 ethyl acetate-hexane to furnish the sulfone as a colorless syrup: ¹H NMR δ-0.02 (3H, s), 0.005 (3H, s), 0.79 (3H, s), 0.87 (9H, s), 1.12 (1H, m), 1.19 (6H, s), 1.12 (1H, m), 1.20 (6H, s), 1.2-1.8 (18H, m), 2.08 (1H, m), 3.09 (1H, dd, J=9.3 and 14.5 Hz), 3.31 (1H, dd, J=3 and 14.5 Hz), 3.97 (1H, brs), 7.58 (3H, m), 7.66 (1H, m), 7.91 2H, m); LR-ES(+) m/z: 600 (M+Na+MeCN), 559 (M+Na); LR-ES(−) m/z: 536 (M),535 (M−H); HR-ES(+): Calcd for C₃₀H₅₂O₄SSi+Na 559.3248; found 559.3253.

### [1R,3aR,4S,7aR]-1-(I.(R)-Benzenesulfonylmethyl-5-methyl-5-trimethylsilanyloxy-hexyl)-4-(tert-butyl-dimethyl-silanyloxy)-7a-methyl-octahydro-indene (17b)

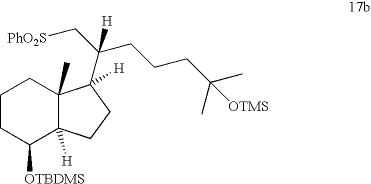

1-(Trimethylsilyl)imidazole (0.146 mL) was added to a solution of 17a (0.145 g, 0.27 mmol) in cyclohexane (2 mL). After 17 h the product was purified by flash chromatography using a stepwise gradient of 1:79 and 1:39 ethyl acetate-hexane to give 17b as colorless residue, 0.157 g 0.258 mmol, TLC (1:9 ethyl acetate-hexane) Rf 0.14. 300 MHz ¹H NMR: δ-0.02 (3H, s), 0.00 (3H, s), 0.87 (12H, s), 1.12 (1H, m), 1.17 (6H, s), 1.2-1.6 (15H, m), 1.6-1.9 (3H, m), 3.08 (2H, m), 3.97 (1H, brs), 7.53-7.70 (3H, m), 7.90 (2H, d, J=7 Hz).

### [1R,3a,4S,7aR]-5(R,S)-Benzenesulfonyl-6(R)-[4-(tert-butyl-dimethyl-silanyloxy)-7a-methyloctahydro-inden-1-yl]-2,10-dimethyl-10-trimethylsilanyloxy-undecane-2,3(R)-diol (18a)

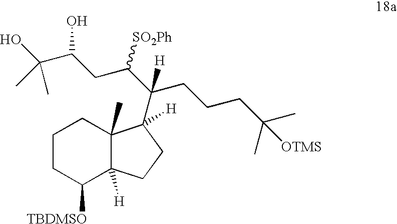

A solution of 17b (0.2589, 0.425 mmol) and diol 21 (0.176 g, 0.638 mmol) in tetrahydrofuran (9 mL) was cooled to −25° C. and 1.6 M butyllithium in hexane (1.4 mL) was added. The temperature was raised to −20° C. and maintained for 3 h then at −10° C. for 2.5 h and 0° C. for 10 min. The mixture was cooled again to −10° C., saturated ammonium chloride solution (5 mL) was added, then equilibrated with ethyl acetate (50 mL) and enough water to dissolve precipitated salts. The aqueous layer was re-extracted with ethyl acetate (15 mL), the combined extracts were dried and evaporated and the residue purified by flash chromatography using a stepwise gradient of 1:6, 1:4, and 1:1 ethyl acetate-hexane to produce 18a as a colorless syrup, 0.212 g, 70%: 300 MHz ¹H NMR: δ 0.00 (3H, s), 0.017 (3H, s), 0.12 (9H, s), 0.81 (3H, s), 0.89 (9H, s), 1.16 (1H, m), 1.19 (12H, m), 1.1-1.6 (20H, m), 1.6-1.8 (2H, m), 3.10 (1H, dd, J=8.4 and 14.7 Hz), 3.30 (1H, m), 3.99 (1H, brs), 7.61 (2H, m), 7.67 (1H, m), 7.93 (2H, m).

### [1R,3aR,4S,7aR]-6(S)-[4-(tert-Butyl-dimethyl-silanyloxy)-7a-methyl-octahydro-inden-1-yl]-2,10-dimethyl-10-trimethylsilanyloxy-undecane-2,3(R)-diol (18b)

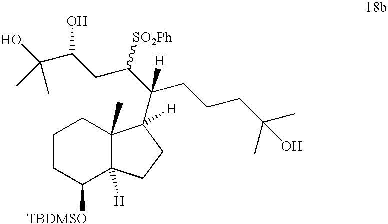

Compound 18a (0.186 mg, 0.262 mmol) was dissolved in 0.5 M oxalic acid dihydrate in methanol (2.5 mL). The solution was stirred for 15 min then calcium carbonate was added (0.5 g) and the suspension stirred overnight then filtered. The filtrate was evaporated to give 18b as a white foam, 0.188 g, 98%: TLC (1:1 ethyl acetate-hexane) Rf 0.06. This material was used in the next step without further purification.

### [1R,3aR,4S,7aR]-6(S)-[4-(tert-Butyl-dimethyl-silanyloxy)-7a-methyl-octahydro-inden-1-yl]-2,10-dimethyl-undecane-2,3(R),10-triol (triol 30a)

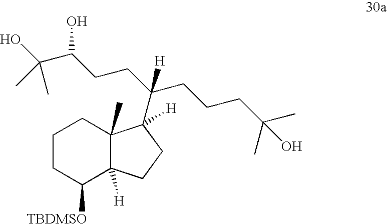

Sodium amalgam (5% sodium, 10.8 g) was added to a vigorously stirred solution of 18b (0.426 g, 0.667 mmol) in a mixture of tetrahydrofuran (15 mL) and methanol (9 mL). The suspension was stirred for 24 h and the reaction monitored by TLC (1:1 ethyl acetate-hexane0 to observe the production of 30a (Rf 0.17). The mixture was diluted with methanol (3 mL), stirred for 5 min then further diluted with water (10 mL), stirred for 2 min and decanted into saturated ammonium chloride solution (25 mL). The aqueous layer was extracted with ethyl acetate (2×20 mL). The combined extracts were washed with pH 7 phosphate buffer (5 mL) then brine (10 mL), dried and evaporated. The residue was purified by flash-chromatography using a stepwise gradient of 1:1 and 2:1 ethyl acetate-hexane to provide 30a as a colorless syrup, 0.244 g, 73%: ¹H NMR: δ 0.006 (3H, s), 0.006 (3H, s), 0.86 (9H, s), 0.92 (3H, s), 1.111 (1H, m), 1.15 (3H, s), 1.21 (9H, s), 1.2-1.75 (21H, m), 1.7-1.85 (3H, m), 1.90 (1H, m), 3.29 (1H, brd), 3.99 (1H, brs); LR-ES(+) m/z: 521 (M+Na), 481 (M−OH); LR-ES(−): m/z 544: (M+CH₂O₂), 543 (M−H+CH₂O₂), 533 M−Cl); HR-ES(+) m/z: Calcd for C₂₉H₅₈O₄Si+Na: 521.3996, found 521.3999.

### [1R,3aR,4S,7aR]-6(S)-(4-Hydroxy-7a-methyl-octahydro-inden-1-yl)-2,10-dimethyl-undecane-2,3(R),10-triol (30b)

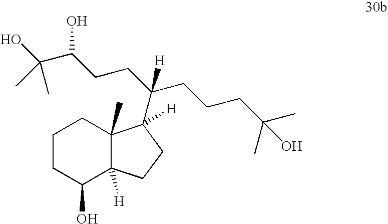

An aqueous fluorosilicic acid solution (3 mL) was added to a stirred solution of 30a (0.240 g, 0.481 mmol) in acetonitrile (12 mL). TLC (ethyl acetate) monitored the reaction. After 2.5 h compound 30b (Rf 0.37) was the predominating species, produced at the expense of less polar 30a. The mixture was equilibrated with ethyl acetate and water (10 mL), the aqueous layer was re-extracted with water (2×10 mL) and the combined extracts were washed with water (6 mL) and brine (2×10 mL) then dried and evaporated. The colorless residue was flash-chromatographed using a stepwise gradient of 1:2, 1:1 and 2:1 ethyl acetate-hexane to elute some unreacted 30a, followed by 30b, obtained as colorless syrup, 0.147 g, 79%: ¹H NMR: 0.94 (3H, s), 1.12 (1H, m), 1.15 (3H, s), 1.21 (9H, s), 1.15-1.7 (20H, m), 1.7-1.9 (5H, m), 1.96 (1H, brd), 3.29 (1H, d, J=9.6 Hz), 4.08 (1H, brs); LR-ES(+): m/z 448: 4+Na+MeCN), 407 (M+Na); LR-ES(−): m/z 419 (M+Cl); HR-ES(+) m/z: Calcd for C₂₃H₄₄O₄+Na: 407.3132, found 407.3135.

### [1R,3aR,4S,7aR]-1-(5-Hydroxy-[(S)-{2-[2-(4-methoxy-phenyl)-5,5-dimethyl-(1,3]dioxolan-(4(R)-yl]-ethyl}-5-methyl-hexyl)-7a-methyl-octahydro-inden.-ol (31)

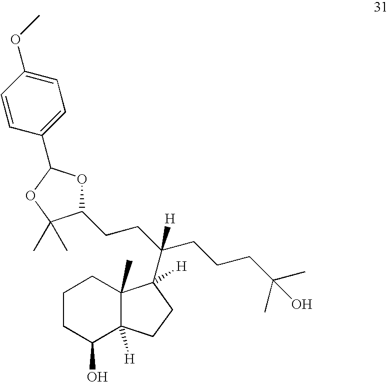

4-Methoxybenzaldehyde dimethyl acetal (60 μL, 0.35 mmol) was added to a solution of 30b (81.2 mg, 0.211 mmol) in dichloromethane (2 mL), followed by a solution (0.2 mL) containing pyridinium tosylate (200 mg) in dichloromethane (10 mL). Reaction progress was followed by TLC (1:2 ethyl acetate-hexane) which showed 4 methoxybenzaldehyde dimethyl acetal (Rf 0.80), 4-methoxybenzaldehyde (Rf 0.65), educt 30b (Rf 0.42) and product 31 (Rf 0.26). After 5¾ h the mixture was stirred for 15 min with saturated sodium hydrogencarbonate solution (5 mL) then equilibrated with ethyl acetate (25 mL). The organic layer was washed with brine (5 mL), dried and evaporated. The residue was flash-chromatographed using a stepwise gradient of 1:3 and 1:2 ethyl acetate-hexane to yield 31 as colorless syrup, 0.106 mg (100%): ¹H NMR: 0.94 (3H, s), 1.19, 1.21 (6H, s each, Me₂COH), 1.23, 1.35 and 1.24, 1.37 (6H, s each, major and minor 5,5-dimethyloxolane diastereomer), 1.1-1.7 (18H, m), 1.7-1.9 (5H, m), 1.9-2.0 (2H, m), 3.65 (1H, m), 3.81 (3H, s), 4.08 (1H, brs), 5.78 and 5.96 (1H, s each, major and minor acetal diastereomer), 6.89 (2H, m), 7.41 (2H, m).

### [1R,3aR,7aR]-1-(5-Hydroxy-1(S)-{2-[2-(4-methoxy-phenyl)-5,5-dimethyl-[1,3]dioxolan-4(R)-yl]-ethyl}-5-methyl-hexyl)-7a-methyl-octahydro-inden-4-one (32)

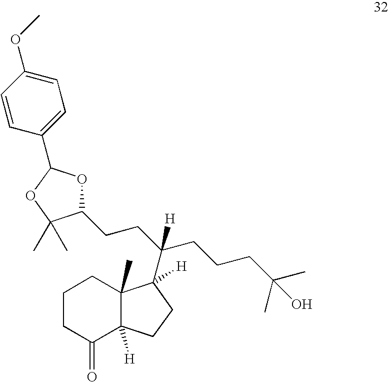

Pyridinium dichromate (230 mg, 0.61 mmol) was added to a stirred mixture containing 31 (0.0838, 0.167 mmol), Celite (185 mg), and dichloromethane (4 mL). The conversion of 31 (Rf 0.31) to 32 (Rf 0.42) was monitored by TLC (1:25 methanol-chloroform) The mixture was diluted with dichloromethane (10 mL) after 2.5 h, then filtered through a layer of silica gel. Filtrate and washings (1:1 dichloromethane-ethyl acetate) were evaporated and the residue chromatographed (1:4 ethyl acetate-hexane) to give ketone 32, 0.0763 g, 91%: ¹H NMR: 0.63 (3H, s), 1.19, 1.21 and 1.23 (6H, s each, Me₂COH), 1.25, 1.36, 1.38 (6H, m,s,s, 5,5-dimethyloxolane diastereomer), 1.1-1.9 (18H, m), 1.9-2.1 (3H, m), 2.1-2.4 (2H, m), 2.45 (1H, m), 3.66 (1H, m), 3.802 and 3.805 (3H, s each), 5.78 and 5.95 (1H, s each, major and minor acetal diastereomer), 6.89 (2H, m), 7.39 (2H, m).

### [1R,3aR,7aR]-1-[4(R),5-Dihydroxy-1(S)-(4-hydroxy-4-methyl-pentyl)-5-methyl-hexyl]-7a-methyl-octahydro-inden-4-one (33)

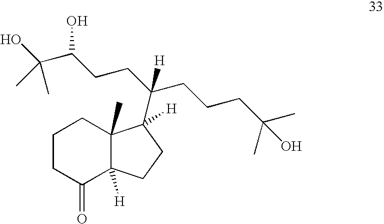

The ketone 32 was stirred in a 1 N oxalic acid solution in 90% methanol. The mixture became homogeneous after a few min. TLC (ethyl acetate) suggested complete reaction after 75 min (Rf 0.24 for 33). Thus, calcium carbonate (0.60 g) was added and the suspension stirred overnight, then filtered. The filtrate was evaporated and flash-chromatographed using a stepwise gradient of 4:1:5 dichloromethane-ethyl acetate-hexane, 1:1 ethyl acetate-hexane, and neat ethyl acetate produce 33 as a colorless residue, 0.060 mg, 94%: ¹H NMR: 0.5 (3H, s), 1.17 (3H, s), 1.22 (6H, s), 1.23 (3H, s), 1.2-1.21 (23H, m), 2.15-2.35 (2H, m), 2.45 (1H, dd, J=7 and 11 Hz), 3.30, 1H, brd).

### [1R,3aR,7aR]-7a-Methyl-1-[5-methyl-1(S)-(4-methyl-4-triethylsilanyloxy-pentyl)-4R),5-bis-triethylsilanyloxy-hexyl]octahydro-inden-4-one (34)

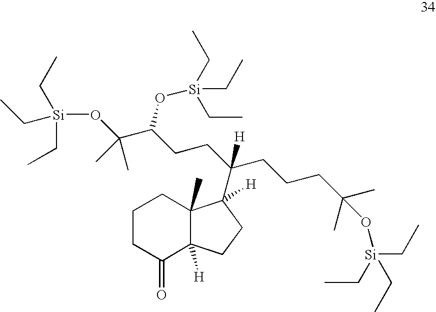

A mixture of 33 (0.055 g, 0.143 mmol), imidazole, (14.9 mg, 1.69 mmol), N,N-dimethylpyridine (6 mg), triethylchlorosilane (0.168 mL, 1 mmol) and N,N-dimethylformamide (1.5 mL) was stirred for 17 h. The reaction was followed by TLC (1:4 ethyl acetate-hexane) and showed rapid conversion to the disilyl intermediate (Rf 0.47). Further reaction proceeded smoothly overnight to give the fully silylated 34 (Rf 0.90). The solution was equilibrated with water (3 mL), equilibrated with ethyl acetate (20 mL), the ethyl acetate layer was washed with water (3×4 mL), dried and evaporated. The residue was flash-chromatographed using a stepwise gradient of hexane and 1:100 ethyl acetate-hexane to yield 34 as a colorless syrup, 0.0813 g, 78.4%: ¹H NMR δ 0.55-0.64 (21H, m), 0.92-0.97 (27H, m), 1.12 (3H, s), 1.18 (3H, s), 1.19 (3H, s), 1.21 (3H, s), 1.1-1.7 (18H, m), 1.9-2.15 (2H, m), 2.15-2.35 (2H, m), 2.43 (1H, dd, J=7.7 and 11 Hz), 3.30 (1H, dd, J=3 and 8.4 Hz).

### [1R,3aR,7aR,4E]-4-{2(Z)-[3(S),5(R)-Bis-(tert-butyl-dimethyl-silanyloxy)-2-methylene-cyclohexylidene]-ethylidene}-7a-methyl-1-[5-methyl-1(S)-(4-methyl-4-triethylsilanyloxy-pentyl)-4(R),5-bis-triethylsilanyloxy-hexyl]-octahydro-indene (35)

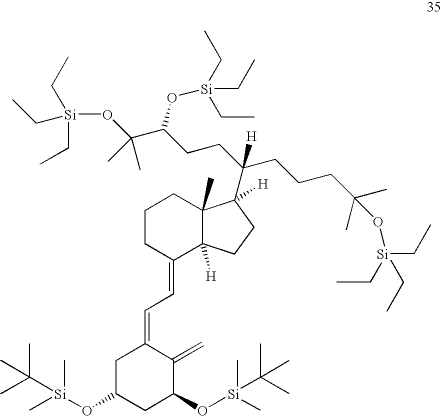

A solution of 1.6 M butyllithium in hexane (0.14 mL) was added to a solution of 28 (0.1308 g, 0.224 mmol) in tetrahydrofuran (1.5 mL) at −70° C. After 10 min a solution of ketone 34 (0.0813 g, 0.112 mmol) in tetrahydrofuran (1.5 mL) was added dropwise over a 15 min period. The ylide color had faded after 3 h so that pH 7 phosphate buffer (2 mL) was added and the temperature allowed to increase to 0° C. The mixture was equilibrated with hexane (30 mL), the organic layer was washed with brine (5 mL), dried and evaporated to give a colorless oil that was purified by flash-chromatography (1:100 ethyl acetate-hexane). Only the band with Rf 0.33 (TLC 1:39 ethyl acetate-hexane) was collected. Evaporation of those fractions gave 35 as colorless syrup, 0.070g, 57%: ¹H NMR 6 0.06 (12H, brs), 0.53-0.64 (21H, m), 0.88 (18H, s), 0.92-0.97 (27H, m), 1.11 (3H, s), 1.177 (3H, s), 1.184 (3H, s), 1.195 (3H, s), 1-1.9 (22H, m), 1.98 (2H, m), 2.22 (1H, m), 2.45 (1H, m), 2.83 (1H, brd, J=13 Hz, 3.27 (1H, d, J=6 Hz), 4.19 (1H, m), 4.38 (1H, m), 4.87 (1H, brs), 5.18 (1H, brs), 6.02 (1H, d, J=11Hz),6.24 (1H, d, J=11 Hz).

### Synthesis of 1,25-Dihydroxy-21(2R,3-dihydroxy-3-methyl-butyl)-20S-Cholecalciferol (2)

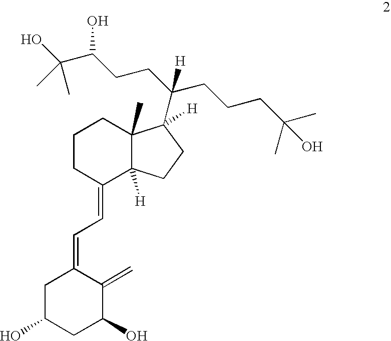

The deprotection reaction of 35 (0.068 g, 0.06238 mmol) in 1M solution of tetrabutylammonium fluoride in tetrahydrofuran, followed by TLC (ethyl acetate), gradually proceeded to give 2 (Rf 0.19). The mixture was diluted with brine (5 mL) after 25 h, stirred for 5 min the equilibrated with ethyl acetate (35 mL) and water (15 mL). The aqueous layer was re-extracted once with ethyl acetate (35 mL), the combined extracts were washed with water (5×10 mL) and brine (5 mL) then dried and evaporated. The residue was flash-chromatographed using a linear gradient of 1:1 and 2:1 ethyl acetate-hexane, and 2:98 methanol-ethyl acetate to give a residue that was taken up in methyl formate and evaporated to a white foam, 30 mg, 93%: [α]_{D}+29.3° (methanol, c 0.34); MHz ¹H NMR δ: 0.55 (3H, s), 1.16 (3K, s), 1.21 (9H, s), 1.1-1.75 (22H, m), 1.80 (2H, m), 1.9-2.1 (5H, m), 2.31 (1H, dd, J=7 and 13 Hz), 2.60 (1H, brd), 284 (1H, m), 3.29 (1H, d, J=9.5 Hz), 4.22 (1H, m), 4.43 (1H, m), 5.00 (1H, s), 5.33 (1H, s), 6.02 (1H, d, J=11 Hz), 6.02 (1H, d, J=11 Hz); LR-ES(−) m/z: 564 (M+H₂CO₂), 563 M−H+H₂CO₂); HR-ES(+) calcd for C₃₂H₅₄O₅+Na: 541.3863; found 541.3854; UVₘₐₓ (ε): 211(15017),265(15850), 204 sh (14127), 245 sh(13747) nm.

### Example 3

### Synthesis of 1,25-Dihydroxy-21-(2R,3-dihydroxy-3-methyl-butyl)-20S-19-nor-cholecalciferol (38)

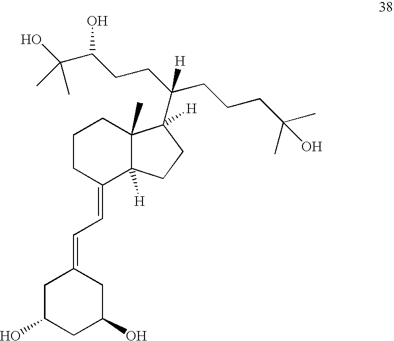

### [1R,3aR,7aR,4E]-4-{2(Z)-[3(S),5(R)-Bis-(tert-butyl-dimethyl-silanyloxy)-cyclohexylidene]-ethylidene}-7a-methyl-1-[5-methyl-1(S)-(4-methyl-4-triethylsilanyloxy-pentyl)-4(R),5-bis-triethylsilanyloxy-hexyl]-octahydro-indene (37)

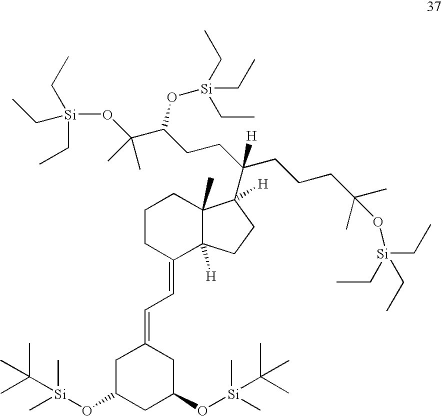

A solution of 1.6 M butyllithium in hexane was added to a solution of 36 in tetrahydrofuran at −70° C. After 10 min a solution of ketone 34 from Example 2 in tetrahydrofuran was added dropwise over a 15 min period. After the ylide color had faded, pH 7 phosphate buffer was added and the temperature allowed to increase to 0° C. The mixture was equilibrated with hexane, the organic layer was washed with brine, dried and evaporated to give a colorless oil that was purified by flash-chromatography (1:100 ethyl acetate-hexane) that gave 37.

1,25-Dihydroxy-21-(2R,3-dihydroxy-3-methyl-butyl)-20S-19-nor-cholecalciferol (38)

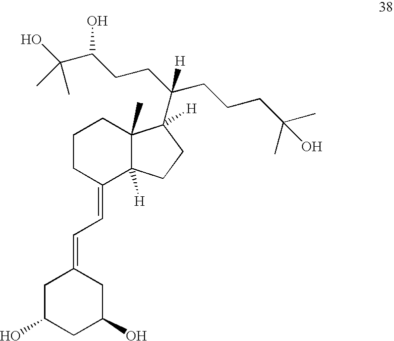

The deprotection reaction of 37 was carried out in 1M solution of tetrabutylammonium fluoride in tetrahydrofuran to give 38. The mixture was diluted with brine after 25 h, stirred for 5 min and then equilibrated with ethyl acetate and water. The aqueous layer was re-extracted once with ethyl acetate, the combined extracts were washed with water and brine, and then dried and evaporated. The residue was flash-chromatographed to give a residue that was taken up in methyl formate and evaporated to yield 38.

### Example 4

### Synthesis of 1,25-dihydroxy-20S-21(3-hydroxy-3-methyl-butyl)-24-keto-19-nor-cholecalciferol (12)

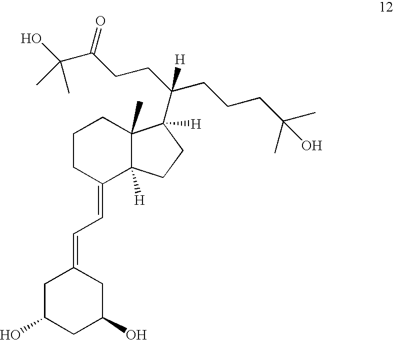

(All Compound Numbers Used in this Example are with Reference to Scheme 6 Above.)

### (R)-6-[(1R,3aR,4S,7aR)-4-(tert-Butyl-dimethyl-silanyloxy)-7a-methyl-octahydro-inden-1-yl]-2-methyl-7-phenylsulfanyl-heptan-2-ol (2)

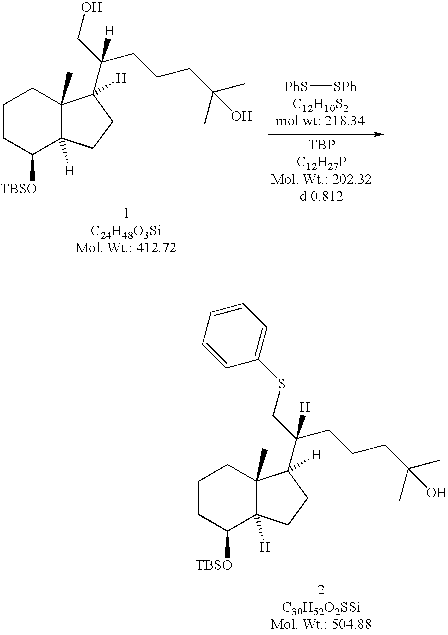

The reaction above was carried out as described in Tet. Lett. 1975, 17: 1409-12. Specifically, a 50 mL round-bottom flask was charged with 1.54 g (3.73 mmol) of (R)-2-[(1R,3aR,4S,7aR)-1(tert-Butyldimethylsilanylox)-7a-methyloctahydroinden-1-yl]-6-methylheptane-1,6-diol (1) (Eur. J. Org. Chem. 2004, 1703-1713) and 2.45 g (1.2 mmol) of diphenylsulfide. The mixture was dissolved in 5 mL of pyridine and 2.27 g (11.2 mmol, 2.80 mL) of tributylphosphine was added. The mixture was stirred overnight and then diluted with 20 mL of toluene and evaporated. The residue was again taken up in toluene and evaporated, the remaining liquid chromatographed on silica gel using stepwise gradients of hexane, 1:39, 1:19 and 1:9 ethyl acetate-hexane to provide the title compound 2 as a syrup, 1.95 g.

### (R)-7-Benzenesulfonyl-6-[(1R,3aR,4S,7aR)-4-(tert-butyl-dimethyl-silanyloxy)-7a-methyl-octahydro-inden-1-yl]-2-methyl-heptan-2-ol (3) and (1R,3aR,4S,7aR)-1-((R)-1-Benzenesulfonylmethyl-5-methyl-5-triethylsilanyloxy-hexyl)-4-(tert-butyl-dimethyl-silanyloxy)-7a-methyl-octahydro-indene (4)

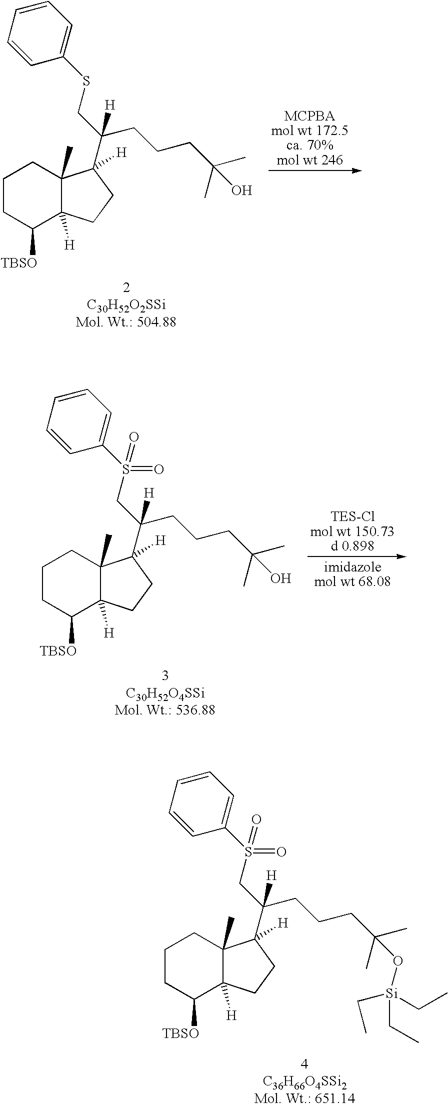

A 500-mL round-bottom flask containing 1.95 g (3.9 mmol) of the crude sulfide 2 was admixed with 84g of dichloromethane (63 mL). The solution was stirred in an ice bath, then 2.77 g (11 mmol) of meta-chloroperbenzoic acid was added in one portion. The suspension was stirred in the ice bath for 40 min then at room temperature for 2 h. The reaction was monitored by TLC (1:19 methanol-dichloromethane). At the end of the reaction period, only one spot at Rf 0.45 observed. Then, 1.68 g (20 mmol) of solid sodium hydrogen carbonate was added to the suspension, the suspension was stirred for 10 min, then 30 mL of water was added in portions and vigorous stirring continued for 5 min to dissolve all solids. The mixture was further diluted with 40 mL of hexane, stirred for 30 min, transferred to a separatory funnel with 41.6 g of hexane. The lower layer was discarded and the upper one was washed with 25 mL of saturated sodium hydrogen carbonate solution, dried (sodium sulfate) and evaporated to give 3.48 g of 3. This material was triturated with hexane, filtered, and evaporated, to leave 3 as a cloudy syrup (2.81 g) that was used directly in the next step.

A 100-mL round bottom flask containing 2.81 g of 3 obtained above, was charged with 30 mL of N,N-dimethylformamide 1.43 g of (21 mmol) of imidazole and 1.75 mL of (10 mmol) of triethylsilyl chloride. The mixture was stirred for 17 h then diluted with 50 g of ice-water, stirred for 10 min, further diluted with 5 mL of brine and 60 mL of hexane. The aqueous layer was re-extracted with 20 mL of hexane, both extracts were combined, washed with 2×30 mL of water, dried, evaporated. This material contained a major spot with Rf 0.12 (1:39 ethyl acetate-hexane) and a minor spot with Rf 0.06. This material was chromatographed on silica gel using hexane, 1:100, 1:79, 1:39 and 1:19 ethyl acetate-hexane as stepwise gradients. The major band was eluted with 1:39 and 1:19 ethyl acetate-hexane to yield 1.83 g of 4.

### (R)-5-Benzenesulfonyl-6-[(1R,3aR,4S,7aR)-4-(tert-butyl-dimethyl-silanyloxy)-7a-methyl-octahydro-inden-1-yl]-10-methyl-2-(R)-methyl-10-triethylsilanyloxy-undecane-2,3-diol (5)

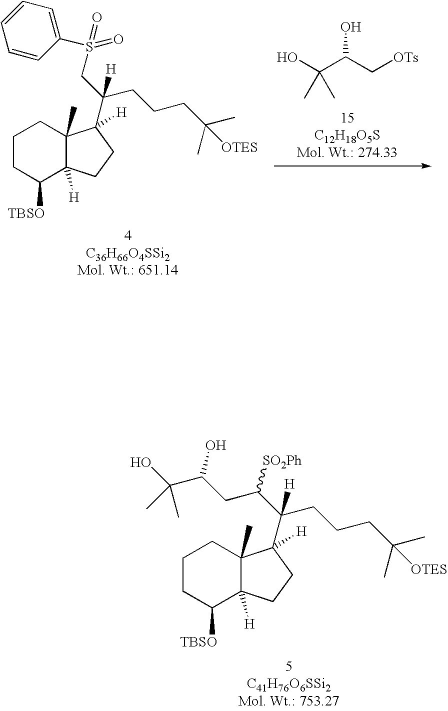

A 100-mL 3-neck round-bottom flask, equipped with magnetic stirrer, thermometer and Claisen adapter with rubber septum and nitrogen sweep, was charged with 1.7636 g of (2.708 mmol) of sulfone 4, 1.114 g of (4.062 mmol) tosylate 15, and 50 mL of tetrahydrofuran freshly distilled from benzophenone ketyl. This solution was cooled to −20° C. and 9.31 mL of a 1.6 M butyllithium solution in hexane was added dropwise at ≦−20° C. The temperature range between −10 and −20° C. was maintained for 5 h. The cooling bath was removed and 50 mL of saturated ammonium chloride solution added followed by 75 mL of ethyl acetate and enough water to dissolve all salts. The organic layer was washed with 15 mL of brine, dried, and evaporated to a colorless oil. This residue was chromatographed on silica gel using hexane, 1:9, 1:6, 1:4 and 1:3 ethyl acetate-hexane as stepwise gradients. The main band was eluted with 1:4 and 1:3 ethyl acetate-hexane to furnish 1.6872 g of compound 5 as colorless syrup.

### (S)-6-[(1R,3aR,4S,7aR)-4-(tert-Butyl-dimethyl-silanyloxy)-7a-methyl-octahydro-inden-1-yl]-10-methyl-2-(R-methyl-10-triethylsilanyloxy-undecane-2,3-diol (6)

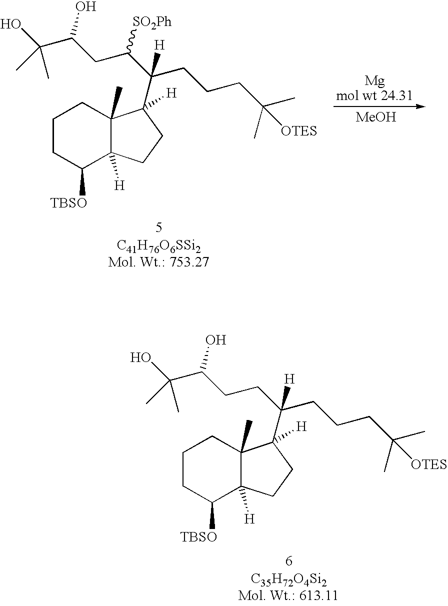

A 25-mL 2-neck round-bottom flask, equipped with magnetic stirrer, thermometer and Claisen adapter with rubber septum and nitrogen sweep, was charged with 1.6872 g (2.238 mmol) of sulfone 5 and 40 mL of methanol. Then 1.25 g (51.4 mmol) of magnesium was added to the stirred solution in two equal portions, in a 30 min time interval. The suspension was stirred for 70 min then another 0.17 g of magnesium and ca. 5 mL of methanol was added and stirring continued 1 h. The mixture was then diluted with 100 mL of hexane and 50 mL of 1 M sulfuric acid was added dropwise to give two liquid phases. The aqueous layer was neutral. The aqueous layer was re-extracted once with 25 mL of 1:1 dichloromethane-hexane. The organic layers were combined then washed once with 15 mL of brine, dried and evaporated. The resulting material was chromatographed on silica gel using hexane, 1:39, 1:19 and 1:9 ethyl acetate-hexane as stepwise gradients. The main band was eluted with 1:9 ethyl acetate-hexane to provide 1.2611 g of 6 as a colorless syrup.

### (S)-6-[(1R,3aR,4S,7aR)-4-(tert-Butyl-dimethyl-silanyloxy)-7a-methyl-octahydro-inden-1-yl]-2,10-dihydroxy-2,10-dimethyl-undecan-3-one (7)

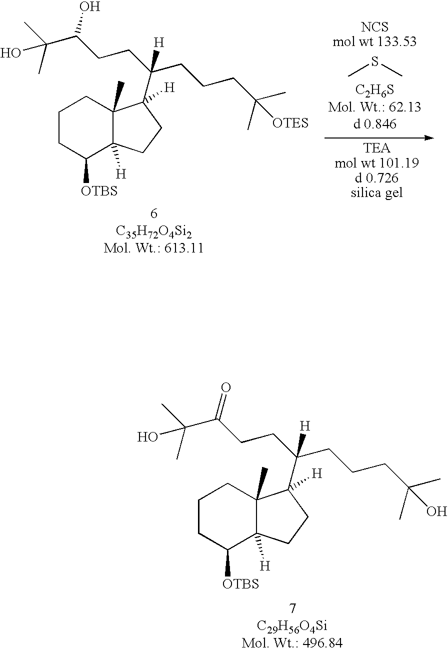

A 25-mL round-bottom flask, equipped with magnetic stirrer, thermometer, Claisen adapter with nitrogen sweep and rubber septum, was charged with 518 mg (3.88 mmol) of N-chlorosuccinamide and 11 mL of toluene. Stir for 5 min (not all dissolved), then cool to 0° C. and add 2.4 mL (4.8 mmol) of a 2M dimethyl sulfide solution in toluene. The mixture was stirred from 5 min then cooled to −30° C. and a solution of 0.7143 g (1.165 mmol) of the diol 6 in 4×1.5 mL of toluene was added dropwise at −30° C. Stirring was continued at this temperature for 1 h. The mixture was then allowed to warm to −10° C. during a 2 h time period then cooled to −17° C. and 3.20 mL (6.4 mmol) of 2 M triethylamine in toluene added dropwise. The mixture was stirred at −17 to −20° C. for 10 min then allowed to warm to room temperature slowly. The mixture was chromatographed on a silica gel column using hexane, 1:79, 1:39, 1:19, 1:9, 1:4, and 1:1 ethyl acetate-hexane as stepwise gradients. The major band was eluted with 1:1 ethyl acetate-hexane providing 0.3428 g of the compound 7 as solids.

### (S)-2,10-Dihydroxy-6-((1R,3aR,4S,7aR)-4-hydroxy-7a-methyl-octahydro-inden-1-yl)-2,10-dimethyl-undecan-3-one (8)

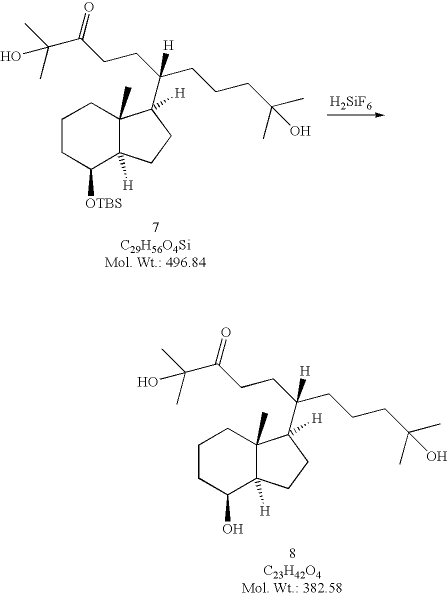

A 25-mL round-bottom flask, equipped with magnetic stirrer was charged with 0.3428 g (0.69 mmol) of the diol 7, was dissolved in 5 mL of acetonitrile then 1.25 mL of fluorosilicic acid solution. After 3 h, the mixture was distributed between 35 mL of ethyl acetate and 10 mL of water, the aqueous layer was re-extracted with 10 mL of ethyl acetate, the organic layers combined, washed with 2×5 mL of water, once with 5 mL of 1:1 brine-saturated sodium hydrogen carbonate solution, dried and evaporated. This material was chromatographed on silica gel using 1:4, 1:3, 1:2, and 1:1 as stepwise gradients furnishing 0.2085 g of the title compound 8.

### (1R,3aR,7aR)-1-[(S)-5-Hydroxy-1-(4-hydroxy-4-methyl-pentyl)-5-methyl-4-oxo-hexyl]-7a-methyl-octahydro-inden-4-one (9)

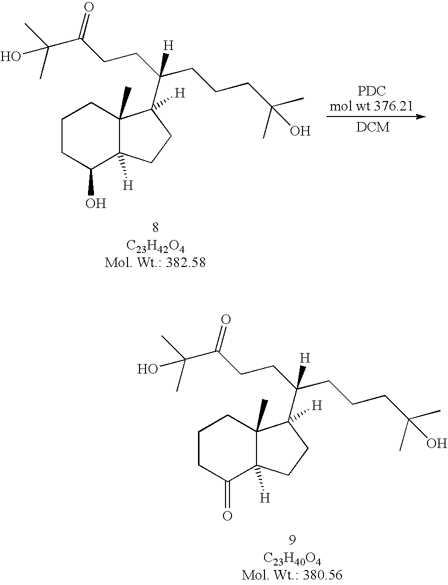

A 25-mL round bottom flask was charged with 0.2153 g (0.56 mmol) of 8, 5 mL of dichloromethane, and 0.20 g of Celite. To this stirred suspension was added, in on portion, 1.00 g (2.66 mmol) of pyridinium dichromate. The reaction stirred for 3 h and the progress was monitored by TLC (1:1 ethyl acetate-hexane). The reaction mixture was diluted with 5 mL of cyclohexane then filtered trough silica gel G. The column was eluted with dichloromethane followed by 1:1 ethyl acetate-hexane until no solute was detectable in the effluent. The effluent was evaporated and the colorless oil. This oil was then chromatographed on a silica gel using 1:4, 1:3, 1:2, 1:1 and 2:1 ethyl acetate-hexane as stepwise gradients to furnish 0.2077 g of the diketone 9.

### (1R,3aR,7aR)-7a-Methyl-1-[(S)-5-methyl-1-(4-methyl-4-trimethylsilanyloxy-pentyl)-4-oxo-5-trimethylsilanyloxy-hexyl]-octahydro-inden-4-one (10)

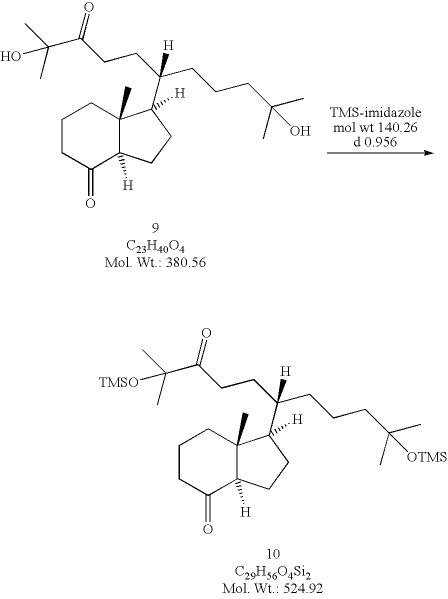

A 25-mL round bottom flask was charged with 0.2077 g (0.545 mmol) of the diketone 9. This material was dissolved in a mixture of 0.5 mL of tetrahydrofuran and 3 mL of cyclohexane. To the resulting mixture was added 0.30 mL (2.0 mmol) of TMS-imidazole. The reaction mixture was diluted with 3 mL of hexane after 10 h then concentrated and chromatographed on silica gel using hexane, 1:79, 1:39, 1:19 and ethyl acetate-hexane as stepwise gradients to provide 0.2381 g of 10 as a colorless oil.

### (S-6-((1R,3aS,7aR)-4-{2-[(R)-3-((R)tert-Butyldimethylsilanyloxy)-5-(tert-butyldimethylsilanyloxy)-cyclohexylidene]-ethylidene}-7a-methyloctahydroinden-1-yl)-2,10-dimethyl-2,10-bis-trimethylsilanyloxyundecan-3-one (11)

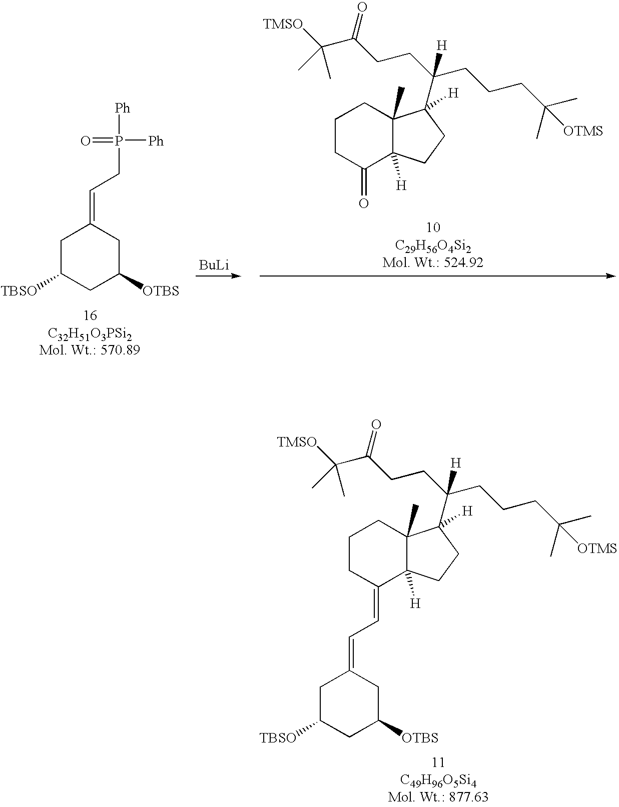

A 15-mL 3-neck pear-shaped flask, equipped with magnetic stirrer, thermometer and a Claisen adapter containing a nitrogen sweep and rubber septum, was charged with 0.2722 g (0.4768 mmol) of [2-[(3R,5R)-3,5-bis(tert-butyldimethylsilanyloxy)cyclohexylidene]ethyl]diphenylphosphine oxide (16) and 2 mL of tetrahydrofuran. The solution was cooled to −70° C. and 0.30 mL of 1.6 M butyllithium in hexane was added. The deep red solution was stirred at that temperature for 10 min then 0.1261 g (0.240 mmol) of the diketone 10, dissolved in 2 mL of tetrahydrofuran was added, via syringe, dropwise over a 10 min period. After 3 h and 15 min, 5 mL of saturated ammonium chloride solution was added at −65° C., the mixture allowed to warm to 10° C. then distributed between 35 mL of hexane and 10 mL of water. The aqueous layer was re-extracted once with 10 mL of hexane, the combined layers washed with 5 ml of brine containing 2 mL of pH 7 buffer, then dried and evaporated. This material was chromatographed on a flash column, 15×150 mm using hexane and 1:100 ethyl acetate-hexane as stepwise gradients to yield 0.1572 g of the title compound 11 as a colorless syrup.

### 1,25-Dihydroxy-20S-21(3-hydroxy-3-methyl-butyl)-24-keto-19-nor-cholecalciferol (12)

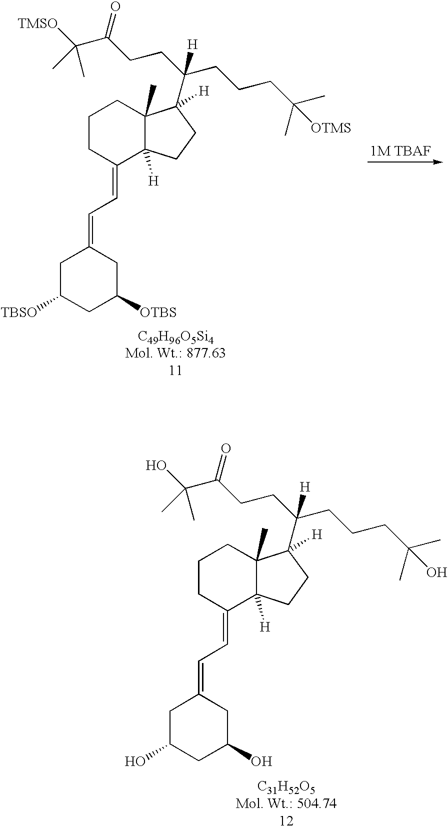

A 15-mL 3-neck round-bottom flask, equipped with magnetic stirrer, was charged with 155 mg (0.17 mmol) of tetrasilyl ether 11. This colorless residue was dissolved is 2 mL of a 1 M solution of tetrabutylammonium fluoride in tetrahydrofuran. After 43 h an additional 0.5 mL of 1 M solution of tetrabutylammonium fluoride solution was added and stirring continued for 5 h. The light-tan solution was the diluted with 5 mL of brine, stirred for 5 min and transferred to a separatory funnel with 50 mL of ethyl acetate and 5 mL of water then re-extraction with 5 mL of ethyl acetate. The organic layers were combined, washed with 5×10 mL of water, 10 mL of brine, dried and evaporated. The resulting residue was chromatographed on a 15×123 mm column using 2:3, 1:1, 2:1 ethyl acetate-hexane, and ethyl acetate as stepwise gradients to provide the 12 as a white solid (TLC, ethyl acetate, Rf 0.23) that was taken up in methyl formate, filtered and evaporated furnishing 0.0753 g of the title compound 12 as a solid substance.

### Example 5

### Synthesis of 1,25-dihydroxy-20S-21(3-hydroxy-3-methyl-butyl)-24-keto-cholecalciferol (14)

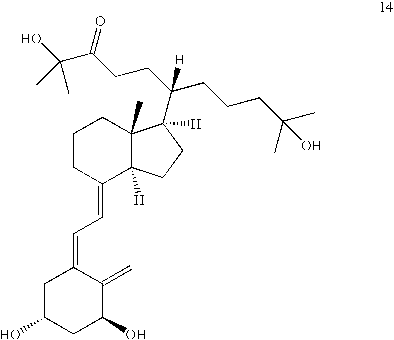

(All Compound Numbers Used in this Example are with Reference to Scheme 6 Above.)

### (S)-6-{(1R,3aS,7aR)-4-[2-[(R)-3-(tert-Butyl-dimethyl-silanyloxy)-5-((S)-tert-butyl-dimethyl-silanyloxy)-2-methylene-cyclohexylidene]-eth-(E)-ylidene]-7a-methyl-octahydro-inden-1-yl}-2,10-dimethyl-2,10-bis-trimethylsilanyloxy-undecan-3-one (13)

Compound 13 was prepared as described for 11 in Example 4 but by reacting 10 with [(2Z)-2-[(3S,5R)-3,5-bis(tert-butyldimethylsilanyloxy)methylenecyclohexylidene]ethyl]diphenylphosphine oxide (17).

### 1,25-Dihydroxy-20S-21(3-hydroxy-3-methyl-butyl)-24-keto-cholecalciferol (14)

Compound 14 was prepared from 13 by deprotecting 13 as described in Example 4 for 12.

### Example 6

### Determination of Maximum Tolerated Dose (MTD) of Vitamin D₃ Analogs

The maximum tolerated dose of the vitamin D₃ compounds of the invention were determined in eight week-old female C57BL/6 mice (3 mice/group) dosed orally (0.1 ml/mouse) with various concentrations of Vitamin D₃ analogs daily for four days. Analogs were formulated in miglyol for a final concentration of 10, 30, 100 and 300/kg when given at 0.1 ml/mouse p.o. daily. Blood for serum calcium assay was drawn by tail bleed on day five, the final day of the study. Serum calcium levels were determined using a colorimetric assay (Sigma Diagnostics, procedure no. 597). The highest dose of analog tolerated without inducing hypercalcemia (serum calcium>10.7 mg/dl) was taken as the maximum tolerated does (MTD). Table 1 shows the relative MID for four vitamin D₃ compounds.

### Example 7

### Immunological Assay of Vitamin D₃ Compounds

Immature dendritic cells (DC) were prepared as described in Romani, N. et al., J. Immunol. Meth. 196:137. IFN-γ production by allogeneic T cell activation in the mixed leukocyte response (MLR) was determined as described in Penna, G., et al., J. Immunol., 164: 2405-2411 (2000).

Briefly, peripheral blood mononuclear cells (PBMC) were separated from buffy coats by Ficoll gradient and the same number (3×105) of allogeneic PBMC from 2 different donors were co-cultured in 96-well flat-bottom plates. After 5 days, IFN-γ production in the MLR assay was measured by ELISA and the results expressed as amount (nM) of test compound required to induce 50% inhibition of IFN-γ production (IC₅₀). The results from the experiment are shown in Table 1. In Table 1, ‘*’ represents good down regulation of INF-γ (e.g., less than 100 IC₅₀ pM), and ‘**’ represents very good down regulation of INF-γ (e.g., greater than 100 IC₅₀ pM).

TABLE 1

| | | HL- | MTD | |
|---|---|---|---|---|
| | VDR | 60(CD14) | (mice) | INF-γ |
| COMPOUND | Binding | ED₅₀ nM | μg/kg | IC₅₀ pM |
| 1,25(OH)₂D₃ | 100.0** | 1.37* | 1.0* | 29.0* |
| 1,25-Dihydroxy-21- | 38.0** | 0.34* | 3.0* | 22.0* |
| (3-hydroxy-3-methyl- | | | | |
| butyl)-cholecalciferol | | | | |
| 1,25-Dihydroxy-21-(2R,3- | 0.95** | 9.30* | 30.0** | 549.0 |
| dihydroxy-3-methyl-butyl)-20S- | | | | |
| cholecalciferol (2) | | | | |
| 1,25-Dihydroxy-21-(2R,3- | 42.10* | 2.17** | 30.0** | 66.0* |
| dihydroxy-3-methyl-butyl)-20R- | | | | |
| cholecalciferol (3) | | | | |
| 1,25-Dihydroxy-21-(2R,3- | — | — | >300 | 3856.0 |
| dihydroxy-3-methyl-butyl)-20S- | | | | |
| 19-nor-cholecalciferol | | | | |
| (38) | | | | |
| 1,25-Dihydroxy-20S-21-(3- | — | — | 30 | 2970.0 |
| hydroxy-3-methyl-butyl)-24- | | | | |
| keto-19-nor-cholecalciferol | | | | |
| (12) | | | | |
| 1,25-Dihydroxy-20S-21-(3- | — | — | 30 | 350 |
| hydroxy-3-methyl-butyl)-24- | | | | |
| keto-cholecalciferol (14) | | | | |
| 1,25-Dihydroxy-21(3-hydroxy- | — | — | .3 | 62 |
| 3-trifluoromethyl-4-trifluoro- | | | | |
| butynyl)-26,27-hexadeutero-19- | | | | |
| nor-20S-cholecalciferol | | | | |
| (39) | | | | |
| 1,25-Dihydroxy-21(3-hydroxy- | — | — | .1 | 62 |
| 3-trifluoromethyl-4-trifluoro- | | | | |
| butynyl)-26,27-hexadeutero- | | | | |
| 20S-cholecalciferol | | | | |
| (40) | | | | |

### Example 8

### Renin Suppression Assay

It has been reported that vitamin D₃ supplementation reduces blood pressure in patients with essential hypertension (Lind, L., et al., Am. J. Hypertens., 2: 20-25 (1989); Pfeifer, M., et al., J. Clin. Endocrinol. Metab. 86: 1633-1637 (2001)) and treatment with 1,25 dihydroxy vitamin D₃ [1,25(OH)₂D₃] reduces blood pressure, plasma renin activity and Angiotensin II levels in patients with hyperparathyroidism (Kimura, Y., et al., Intern Med. 38: 31-35 (1999); Park, C. W., et al., Am. J. Kidney Dis. 33: 73-81 (1999)). Subsequently, it was shown that 1,25(OH)₂D₃ is a negative regulator of the renin-angiotensin system (Li, Y. C., et al., J. Clin. Invest. 110(2): 229-238 (2002)). In other words, 1,25(OH)₂D₃ suppresses expression of renin.

Accordingly, a renin suppression assay (Li, Y. C., et al., J. Clin. Invest. 110(2): 229-238 (2002)) was used to compare the renin suppression activity of certain Gemini vitamin D₃ compounds of the invention with that of 1,25(OH)₂D₃. In accordance with this literature assay, 12 Gemini compounds according to the invention were used to treat As4.1hVDR cells at 10⁻¹⁰, 10⁻⁹ and 10⁻⁸ M for 24 hours. Renin mRNA was quantified by Northern blots. The same cells were treated with 1,25(OH)₂D₃ under the same conditions as a control.

The data are summarized in Table_below. The structures and names of the Gemini compounds tested are shown after Table 2. The data indicate that that Gemini compounds 1, 3, 5 and 11 had renin suppression activity comparable to that of 1,25(OH)₂D₃ and Gemini compounds 2, 4, 6 and 10 were more potent in suppressing renin expression than 1,25(OH)₂D₃.

TABLE 2

| Gemini Compounds | Suppressive Activity* |
|---|---|
| (1) | ++ |
| (2) | ++ |
| (3) | +++ |
| (4) | +++ |
| (5) | ++ |
| (6) | +++ |
| (7) | +/− |
| (8) | +/− |
| (9) | +/− |
| (10) | +++ |
| (11) | ++ |
| (38) | − |

*indicates no activity; +/− and + indicate less active than 1,25-dihydroxy-
vitamin D₃; ++ indicates as active as 1,25-dihydroxyvitamin D3; and +++
indicates more active that 1,25-dihydroxyvitamin D₃.

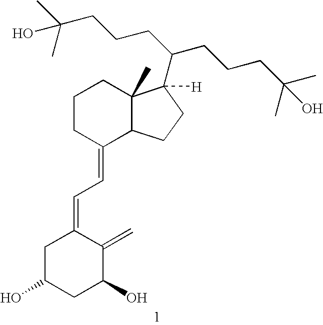

1,25-Dihydroxy-21-
(3-hydroxy-3-methyl-
butyl)-cholecalciferol

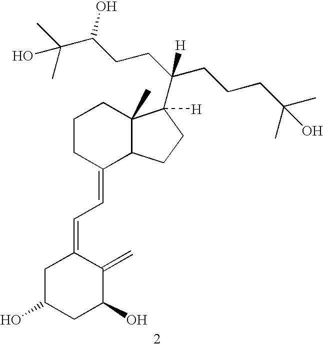

1,25-Dihydroxy-21-
(2R,3-dihydroxy-3-methyl-butyl)-
-20S-cholecalciferol

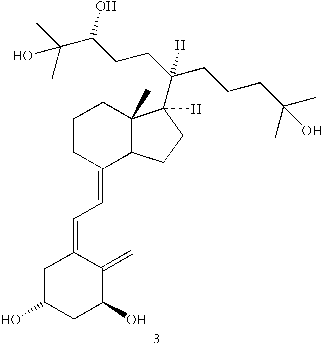

1,25-Dihydroxy-21-
(2R,3-dihydroxy-3-methyl-butyl)-
-20R-cholecalciferol

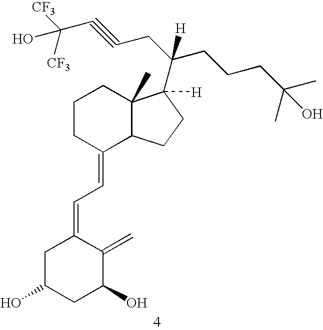

1,25-Dihydroxy-21-
(3-hydroxy-3-trifluoromethyl-4-
trifluoro-butynyl)-20S-cholecalciferol

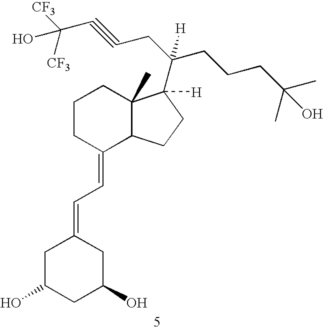

1,25-Dihydroxy-21-
(3-hydroxy-3-trifluoromethyl-4-trifluoro-
butynyl)-20R-19-nor-cholecalciferol

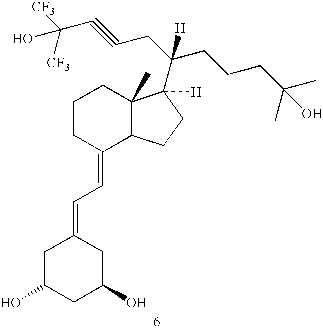

1,25-Dihydroxy-21-
(3-hydroxy-3-trifluoromethyl-4-
trifluoro-butynyl)-20S-19-nor-cholecalciferol

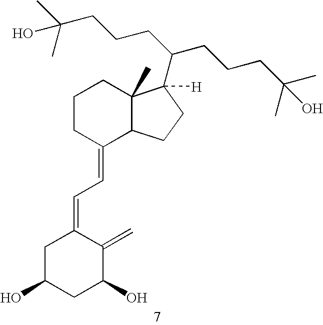

1,25-Dihydroxy-21-
(3-hydroxy-3-methyl-butyl)-3-
epi-cholecalciferol

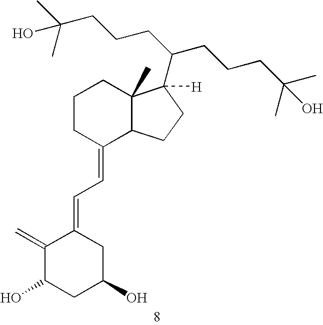

1,25-Dihydroxy-21-
(3-hydroxy-3-methyl-butyl)-
5,6-trans-cholecalciferol

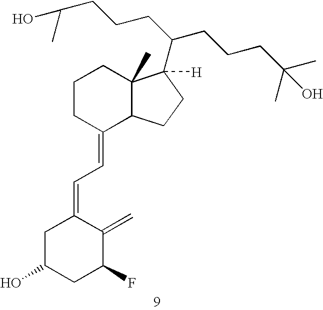

1α-Fluoro-25-hydroxy-21-
(3-hydroxy-3-methyl-butyl)-
cholecalciferol

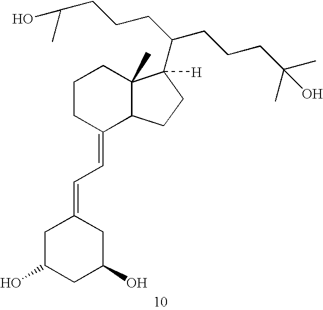

1,25-Dihydroxy-21-
(3-hydroxy-3-methyl-
butyl)-19-nor-cholecalciferol

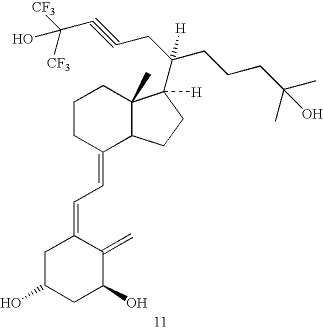

1,25-Dihydroxy-21-
(3-hydroxy-3-trifluoromethyl-4-trifluorobutynyl)-
20R-cholecalciferol

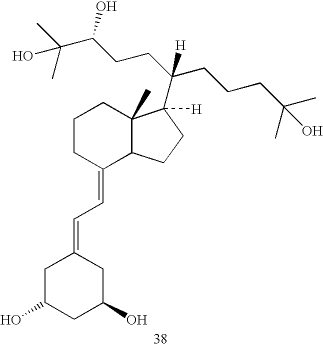

1,25-Dihydroxy-21-
(2R,3-dihydroxy-3-methyl-butyl)-
20S-19-nor-cholecalciferol

### Example 9

### Proliferation Assay Using Bladder Cancer Cell Lines

Bladder cancer cell lines (T24, RT112, HT1376 and RT4 are human bladder cancer cell lines; NHEK are normal human keratinocytes) were obtained from the European Collection of Cell Cultures (Salisbury, UK). Cells were plated at 3×103 per well, in flat bottomed 96-well plates in 100 0d of DMEM medium containing: 5% Fetal Clone 1, 50 μg/l gentamicin, 1 mM sodium pyruvate and 1% non-essential amino acids. After culturing for 24 h at 37° C. in 5% CO2, to allow cells to adhere to the plates, VDR ligands (compounds 2, 3, 12, 14, 38, 39 and 40) were added at concentrations ranging from 100 μM to 0.3 μM in 100 μl of above-mentioned complete medium. After a further 72 h of culture, cell proliferation was measured using a fluorescence-based proliferation assay kit. (CyQuant Cell Proliferation Assay Kit, Molecular Probes, Eugene, Oreg., USA). The IC₅₀ was calculated from the regression curve of the titration data. The results are shown in Table 3.

TABLE 3

| | ECV | RT112 | HT 1376 | RT4 | NHEK |
|---|---|---|---|---|---|
| Compound | (μm) | (μM) | (μM) | (μM) | (μM) |
| 1,25-Dihydroxy-21-(2R,3-dihydroxy-3- | 69.4 | 1 | — | 62 | 37.2 |
| methyl-butyl)-20S-cholecalciferol (2) | | | | | |
| 1,25-Dihydroxy-21-(2R,3-dihydroxy-3- | 57.9 | 25 | 98 | 71 | 46.5 |
| methyl-butyl)-20R-cholecalciferol (3) | | | | | |
| 1,25-Dihydroxy-21-(2R,3-dihydroxy-3- | 99.2 | 44 | >100 | 72 | 57 |
| methyl-butyl)-20S-19-nor- | | | | | |
| cholecalciferol | | | | | |
| (38) | | | | | |
| 1,25-Dihydroxy-20S-21-(3-hydroxy-3- | — | >30 | — | 2.6 | — |
| methyl-butyl)-24-keto-19-nor- | | | | | |
| cholecalciferol | | | | | |
| (12) | | | | | |
| 1,25-Dihydroxy-20S-21-(3-hydroxy-3- | — | 25.5 | — | 8.3 | — |
| methyl-butyl)-24-keto-cholecalciferol | | | | | |
| (14) | | | | | |
| 1,25-Dihydroxy-21(3-hydroxy-3- | — | 7 | — | 2.2 | — |
| trifluoromethyl-4-trifluoro-butynyl)- | | | | | |
| 26,27-hexadeutero-19-nor-20S- | | | | | |
| cholecalciferol | | | | | |
| (39) | | | | | |
| 1,25-Dihydroxy-21(3-hydroxy-3- | — | 6.4 | — | 2.2 | — |
| trifluoromethyl-4-trifluoro-butynyl)- | | | | | |
| 26,27-hexadeutero-20S-cholecalciferol | | | | | |
| (40) | | | | | |
| 1,25 dihydroxycholecalciferol | 54.6 | 19 | 50 | 45 | 4.9 |

### Example 10

### Immunological Assay for Modulation of ILT3 by VDR Agonists

### I. Methods

Immature dendritic cells (DC) were prepared as described in Romani, N. et al., (1996) J. Immunol. Meth. 196:137). Briefly, peripheral blood mononuclear cells (PBMCs) were obtained from a buffy coat by ficoll-hypaque gradient (Pharmacia Biotec AB, Upsala, Sweden) and monocytes isolated from PBMCs by negative selected with a monocyte isolation kit (Milteny, Biotech, Bergish Gladblach, Germany). After isolation, monocytes were cultured for 6 to 7 days at a cell density of 1-2×106 in RPMI medium supplemented with 5% FetalClone I (Hyclone Laboratories, Logan, Utah) 2 mM L-glutamine, 50 mg/ml gentamicin, 1 mM sodium pyruvate and 1% nonessential amino acids, containing 800 U/ml GM-CSF (Mielogen 300, Schering-Plough) and 10 ng/ml interleukin (IL)-4 (PharMingen, San Diego, Calif.). Every other day, approximately 20% of the medium was removed and replaced by the same volume of fresh medium containing GM-CSF and IL-4.

After 6 to 7 days of culture, non-adherent cells (representing immature Dendritic Cells) were harvested and cultured for 24 hours in the presence of graded doses of vitamin D compounds—1,25(OH)₂D₃ (i), 1,24R,25-trihydroxy-20R-21-(3-hydroxy-3-methylbutyl) cholecalciferol (ix) (compound 2 herein) and other vitamin D compounds (x) to (xv); and the compounds Mycophenolate Mofetil (F) and Dexamethasone (DEX).

Immunoglobulin-like transcript-3 upregulation was evaluated by flow cytometric analysis. Briefly, cells were preincubated with 200 mg/ml human IgG (Sigma Chemical, St. Louis, Mo.) and subsequently stained with anti-human ILT3 antibody (see, e.g., Cella, M. et al. (1997) J. Exp. Med. 185:1743) followed by anti-mouse IgG-phycoerithryn (Jackson). Cells were then analyzed with a FACScanR flow cytometer using a Cell QuestR software program (both from Beckton Dickinson, Mountain View, Calif.).

### II. Results

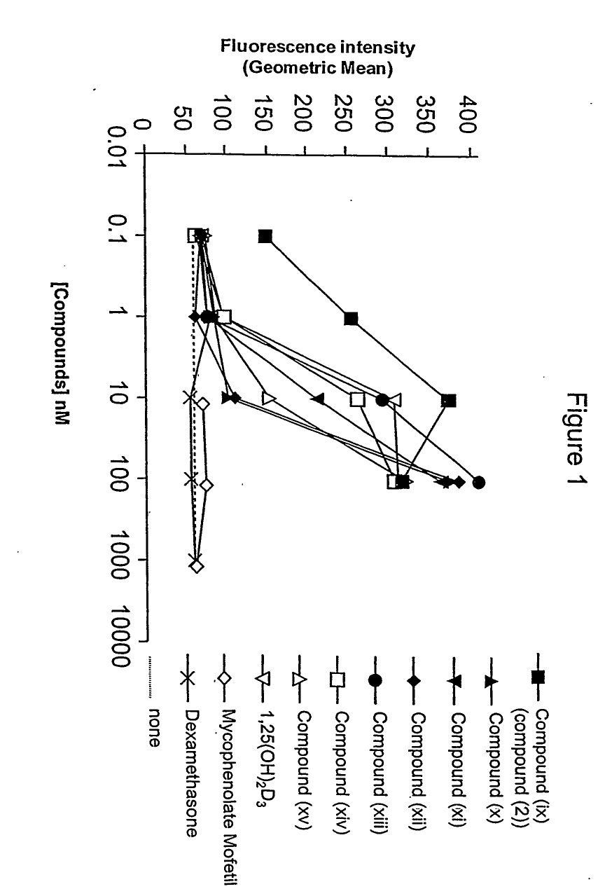

Incubation of monocyte-derived immature dendritic cells with compounds (ix) to (xv) upregulated the expression of ILT3 on their cell surface (FIG. 1). These data show that all the VDR agonists tested upregulate ILT3 expression. Notably, treatment of cells with compound (ix) produced the greatest upregulation of ILT3, at low doses of compound, with a mean fluorescence intensity of 260 at 1 nM of compound.

### Example 11

### Comparison of VDR Agonists with MMF aid DEX

The results shown in FIG. 1 demonstrate that all the VDR agonists tested upregulate ILT3 expression on monocyte-derived immature dendritic cells. Conversely, Mycophenolate Mofetil and Dexamethasone, agents that also target dendritic cells, fail to upregulate ILT3 expression at any concentration tested, up to 1000 nM. In particular, compound 1,24R,25-trihydroxy-20R-21-(3-hydroxy-3-methylbutyl) herein) shows very favourable upregulation in relation to Mycophenolate Mofetil and Dexamethasone.

### Example 12

Soft Gelatin Capsule Formulation I

| Item | Ingredients | mg/Capsule |
|---|---|---|
| 1. | Compound 3 from Example 1 | 10.001-0.02 |
| 2. | Butylated Hydroxytoluene (BHT) | 0.016 |
| 3. | Butylated Hydroxyanisole (BHA) | 0.016 |
| 4. | Miglyol 812 qs. | 160.0 |

### Manufacturing Procedure:

1. BHT and BHA is suspended in Miglyol 812 and warmed to about 50° C. with stirring, until dissolved.

2. A Gemini vitamin D₃ compound of the invention is dissolved in the solution from step 1 at 50° C.

3. The solution from Step 2 is cooled at room temperature.

4. The solution from Step 3 is filled into soft gelatin capsules.

Note: All manufacturing steps are performed under a nitrogen atmosphere and protected from light.

### Example 13

Soft Gelatin Capsule Formulation II

| Item | Ingredients | mg/Capsule |
|---|---|---|
| 1. | Compound 3 from Example | 10.001-0.02 |
| 2. | di-α-Tocopherol | 0.016 |
| 3. | Miglyol 812 qs. | 160.0 |

### Manufacturing Procedure:

1. Di-α-Tocopherol is suspended in Miglyol 812 and warmed to about 50° C. with stirring, until dissolved.

2. A Gemini vitamin D₃ compound of the invention.

3. The solution from Step 2 is cooled at room temperature.

4. The solution from Step 3 is filled into soft gelatin capsules.

### INCORPORATION BY REFERENCE

The contents of all references (including literature references, issued patents, published patent applications, and co-pending patent applications) cited throughout this application are hereby expressly incorporated herein in their entireties by reference.

### EQUIVALENTS

Those skilled in the art will recognize, or be able to ascertain using no more than routine experimentation, many equivalents of the specific embodiments of the invention described herein. Such equivalents are intended with be encompassed by the following claims.

1. A vitamin D₃ compound having formula I: 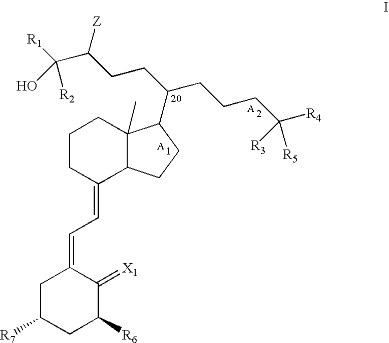
wherein:
A₁ is a single or double bond;
A₂ is a single, a double or a triple bond;
R₁, R₂, R₃ and R₄ are each independently C₁-C₄ alkyl, C₁-C₄ deuteroalkyl, hydroxyalkyl, or haloalkyl;
R₅, R₆ and R₇ are each independently hydroxyl, OC(O)C₁-C₄ alkyl, OC(O)hydroxyalkyl, or OC(O)haloalkyl;
the configuration at C₂₀ is R or S;
X₁ is H₂ or CH₂;
Z is hydrogen when at least one of R₁ and R₂ is C₁-C₄ deuteroalkyl and at least one of R₃ and R₄ is haloalkyl or when at least one of R₁ and R₂ is haloalkyl and at least one of R₃ and R₄ is C₁-C₄ deuteroalkyl; or Z is —OH, ═O, —SH, or —NH₂;
and pharmaceutically acceptable esters, salts, and prodrugs thereof.

2. The compound of claim 1, wherein A₁ is a single bond.

3. The compound of claim 1, wherein A₂ is a single bond.

4. The compound of claim 1, wherein A₂ is a triple bond.

5. The compound of claim 1, wherein R₁, R₂, R₃, and R₄ are each independently methyl or ethyl.

6. The compound of claim 1, wherein R₁, R₂, R₃, and R₄ are each independently C₁-C₄ deuteroalkyl or haloalkyl.

7. The compound of claim 1, wherein R₅ is hydroxyl.

8. The compound of claim 7, wherein R₆ and R₇ are hydroxyl.

9. The compound of claim 7, wherein R₆ and R₇ are each OC(O)C₁-C₄ alkyl.

10. The compound of claim 9, wherein R₆ and R₇ are each acetyloxy.

11. The compound of claim 1, wherein X₁ is H₂.

12. The compound of claim 1, wherein X₁ is CH₂.

13. The compound of claim 1, wherein Z is hydrogen when at least one of R₁ and R₂ is C₁-C₄ deuteroalkyl and at least one of R₃ and R₄ is haloalkyl or when at least one of R₁ and R₂ is haloalkyl and at least one of R₃ and R₄ is C₁-C₄ deuteroalkyl; Z is —OH, ═O, —SH, or —NH₂ when X₁ I is CH₂; Z is —OH, ═O, —SH, or —NH₂ when X₁ I is H₂ and the configuration at C₂₀ is S; or Z is ═O, —SH, or —NH₂ when X₁ I is H₂ and the configuration at C20 is R.

14. The compound of claim 1, wherein Z is hydrogen.

15. The compound of claim 13, wherein Z is —OH.

16. The compound of claim 1, wherein Z is ═O.

17. The compound of claim 1, wherein X₁ I is CH₂; A₂ is a single bond; R₁, R₂, R₃, and R₄ are each independently methyl or ethyl; and Z is —OH.

18. The compound of claim 1, wherein X₁ I is CH₂; A₂ is a single bond; R₁, R₂, R₃, and R₄ are each independently methyl or ethyl; and Z is ═O.

19. The compound of claim 1, wherein X₁ I is H₂; A₂ is a single bond; R₁, R₂, R₃, and R₄ are each independently methyl or ethyl; the configuration at C₂₀ is S; and Z is —OH.

20. The compound of claim 1, wherein X₁ I is H₂; A₂ is a single bond; R₁, R₂, R₃, and R₄ are each independently methyl or ethyl; and Z is ═O.

21. The compound of claim 17, wherein R₁, R₂, R₃, and R₄ are each methyl.

22. The compound of claim 1, wherein X₁ I is H₂; A₂ is a triple bond; R₁ and R₂ are each C₁-C₄ deuteroalkyl; R₃ and R₄ are each haloalkyl; and Z is hydrogen.

23. The compound of claim 1, wherein X₁ I is CH₂; A₂ is a triple bond; R₁ and R₂ are each C₁-C₄ deuteroalkyl; R₃ and R₄ are each haloalkyl; and Z is hydrogen.

24. The compound of claim 22 [or 23], wherein R₁ and R₂ are each deuteromethyl and R₃ and R₄ are each trifluoromethyl.

25. The compound of claim 21, wherein said compound is 1,25-Dihydroxy-21-(2R,3-dihydroxy-3-methyl-butyl)-20R-cholecalciferol:

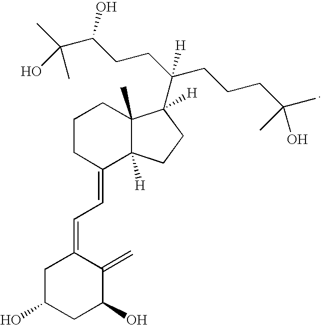

26. The compound of claim 21, wherein said compound is 1,25-Dihydroxy-21-(2R,3-dihydroxy-3-methyl-butyl)-20S-cholecalciferol:

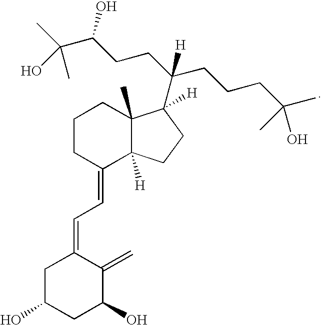

27. The compound of claim 21, wherein said compound is 1,25-Dihydroxy-21-(2R,3-dihydroxy-3-methyl-butyl)-20S-19-nor-cholecalciferol:

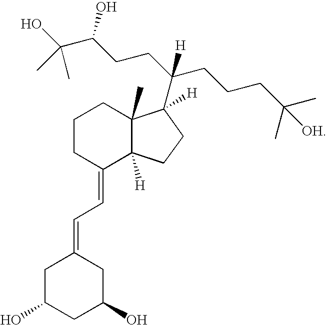

28. The compound of claim 21, wherein said compound is 1,25-Dihydroxy-20S-21-(3-hydroxy-3-methyl-butyl)-24-keto-19-nor-cholecalciferol:

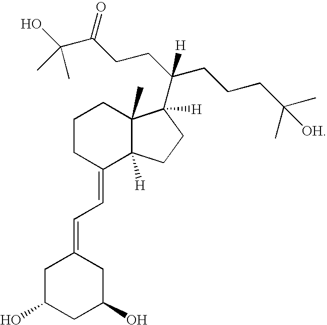

29. The compound of claim 21, wherein the compound is 1,25-Dihydroxy-20S-21-(3-hydroxy-3-methyl-butyl)-24-keto-cholecalciferol:

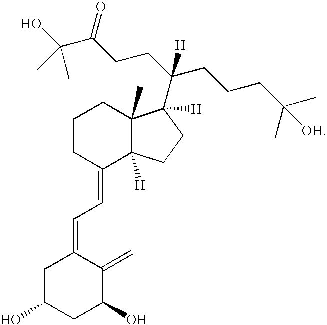

30. The compound of claim 24, wherein the compound is 1,25-Dihydroxy-21(3-hydroxy-3-trifluoromethyl-4-trifluoro-butynyl)-26,27-hexadeutero-19-nor-20S-cholecalciferol:

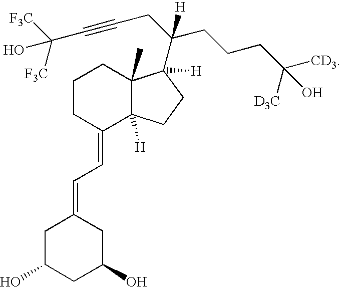

31. The compound of claim 24, wherein the compound is 1,25-Dihydroxy-21(3-hydroxy-3-trifluoromethyl-4-trifluoro-butynyl)-26,27-hexadeutero-20S-cholecalciferol:

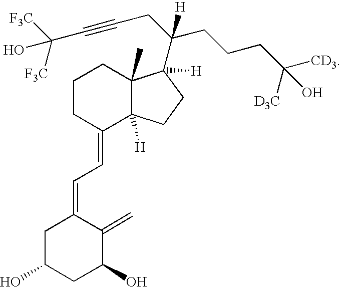

32. The compound of claim 1, wherein the haloalkyl is fluoroalkyl.

33. The compound of claim 32, wherein the fluoroalkyl is fluoromethyl or trifluoromethyl.

34. A method for treating a subject for a vitamin D₃ associated state, comprising administering to said subject an effective amount of a vitamin D₃ compound of claim 1, such that said subject is treated for said vitamin D₃ associated state.

35. The method of claim 34, wherein said vitamin D₃ associated state is a disorder characterized by an aberrant activity of a vitamin D₃-responsive cell.

36. A method for treating a subject for a urogenital disorder, comprising administering to said subject an effective amount of a vitamin D₃ compound of claim 1, such that said subject is treated for said urogenital disorder.

37-46. (canceled)

47. The method of claim 35, wherein said disorder is selected from the group consisting of a disorder comprising an aberrant activity of a hyperproliferative skin cell, a disorder comprising an aberrant activity of an endocrine cell, secondary hypelparathyroidism, a disorder comprising an aberrant activity of a bone cell, cirrhosis, chronic renal disease, neoplastic disease, neuronal loss and a disorder characterized by an aberrant activity of a vitamin D₃-responsive smooth muscle cell.

48-63. (canceled)

64. A method of inhibiting transplant rejection in a subject comprising administering to said subject a vitamin D3 compound of claim 1 in an amount effective to modulate the expression of an ILT3 surface molecule, thereby inhibiting transplant rejection in said subject.

65-77. (canceled)

78. A method of ameliorating a deregulation of calcium and phosphate metabolism, comprising administering to a subject a therapeutically effective amount of a vitamin D₃ compound of claim 1, so as to ameliorate the deregulation of the calcium and phosphate metabolism.

79. (canceled)

80. A method of modulating the expression of an immunoglobulin-like transcript 3 (ILT3) surface molecule in a cell, comprising contacting said cell with a vitamin D₃ compound of claim 1 in an amount effective to modulate the expression of an immunoglobulin-like transcript 3 (ILT3) surface molecule in said cell.

81. (canceled)

82. A method of inducing immunological tolerance in a subject, comprising administering to said subject a vitamin D₃ compound of claim 1 in an amount effective to modulate the expression of an ILT3 surface molecule, thereby inducing immunological tolerance in said subject.

83-84. (canceled)

85. A method for modulating immunosuppressive activity by an antigen-presenting cell, comprising contacting an antigen-presenting cell with a vitamin D₃ compound of claim 1 in an amount effective to modulate ILT3 surface molecule expression, thereby modulating said immunosuppressive activity by said antigen-presenting cell.

86-88. (canceled)

89. The method of claim 34, wherein said subject is a mammal.

90. The method of claim 89, wherein said subject is a human.

91. The method of claim 34 wherein said vitamin D₃ compound is administered in combination with a pharmaceutically acceptable carrier.

92-97. (canceled)

98. A pharmaceutical composition, comprising an effective amount a vitamin D₃ compound of claim 1 and a pharmaceutically acceptable carrier.

99. The pharmaceutical composition of claim 98, wherein said effective amount is effective to treat a vitamin D₃ associated state.

100. (canceled)

101. A packaged formulation comprising a pharmaceutical composition comprising a compound recited in claim 1, and instructions for use in the treatment of a vitamin D₃ associated state.

102. The packaged formulation of claim 101, wherein said compound is present in an amount effective to treat a vitamin D₃ associated state.

103. (canceled)
